(19) United States
(12) Patent Application Publication
(10) Pub. No.: US 2026/0327342 A1
(43) Pub. Date: Oct. 22, 2026

(54) MONOCLONAL ANTIBODIES TARGETING CONFORMATION-SPECIFIC EPITOPES OF IMMUNOGLOBULIN LIGHT CHAINS OF THE KAPPA SUBCLASS

(71) Applicants: PARADOX IMMUNOTHERAPEUTICS INC., Toronto (CA); Eli Lilly and Company, Indianapolis, IN (US)

(72) Inventors: Yulong SUN, Maple (CA); Natalie GALANT, Kleinburg (CA); Julian DAVIES, San Diego, CA (US); Victoria ORTEGA, San Diego, CA (US); Cheng WANG, San Diego, CA (US)

(21) Appl. No.: 19/643,491

(22) Filed: Apr. 9, 2026

Related U.S. Application Data
(60) Provisional application No. 63/787,187, filed on Apr. 11, 2025.

(51) Int. Cl. C07K 16/42; A61K 39/00; A61P 7/00; G01N 33/68

(52) U.S. Cl. CPC C07K 16/4241; A61P 7/00; G01N 33/6854; A61K 2039/505; C07K 2317/14; C07K 2317/565; C07K 2317/92; G01N 2333/70503

(57) ABSTRACT

Provided herein are anti-human κ light chain antibodies or human κ light chain binding antibody fragments that bind an epitope specific of a misfolded human κ light chain. Also provided are polynucleotides encoding the same, and compositions comprising anti-human κ light chain antibodies or human κ light chain binding antibody fragments. The anti-human κ light chain antibodies or human κ light chain binding antibody fragments are useful for measuring human κ light chain in a biological sample comprising contacting the sample with the anti-human κ light chain antibodies or human κ light chain binding antibody fragments. The anti-human κ light chain antibodies or human κ light chain binding antibody fragments are also useful for reducing κ light chain aggregates. Additionally, the anti-human κ light chain antibodies or human κ light chain binding antibody fragments are useful for treating disorders or conditions associated with κ light chain aggregates.

### CROSS-REFERENCE

This application claims the benefit of U.S. Provisional Application No. 63/787,187, filed on Apr. 11, 2025, which is incorporated herein by reference in its entirety.

### REFERENCE TO A SEQUENCE LISTING

The instant application contains a Sequence Listing which has been submitted electronically in XML format and is hereby incorporated by reference in its entirety. Said XML copy, created on Apr. 1, 2026, is named 60070-704_601_SL.xml and is 30,537 bytes in size.

### BACKGROUND

Light chain amyloidosis (AL amyloidosis) is a life-threatening secondary condition that is associated with the primary spectrum disorder, plasma cell dyscrasia. It is a widely underdiagnosed disease caused by the misfolding of free immunoglobulin light chains (LC) which can deposit as light chain amyloid (AL) in heart, kidney, and nervous system. AL amyloidosis occurs in ~15% of plasma cell dyscrasias, where a single plasma cell clone undergoes unregulated growth, leading to aberrant production and secretion of a large excess of a monoclonal free immunoglobulin kappa (κ) or lambda (λ) light chain protein into the circulation. This increase in light chain protein concentration can lead to a particular type of aggregation and accumulation known as amyloid deposition. Deposition and infiltration of amyloid in vital organs, such as the heart, kidneys and nervous system results in restrictive cardiomyopathy, nephrotic syndrome, hepatomegaly, peripheral neuropathy, and ultimately death.

AL amyloidosis is the most common type of systemic amyloidosis. With an incidence of 9-14 people per million per year in the Western world. Its prevalence is likely to be underestimated as most patients remain undiagnosed until post-mortem. The Amyloidosis Foundation estimates that more than 35,000-45,000 patients in the USA and EU are suffering from this disease, and there are approximately ~12,000 and ~18,600 new AL patients diagnosed in the US and EU each year, respectively. The life expectancy of patients diagnosed with AL amyloidosis depends on extent of organ involvement. For patients with cardiac amyloid deposition (approximately 50%), the prognosis is dismal with a median survival of <6 months from the onset of heart failure symptoms if patients remain untreated.

### SUMMARY

The anti-human κ light chain antibodies described herein are useful and advantageous in that the antibodies and antibody fragments specifically bind to an epitope unique to misfolded κ light chains while largely not recognizing natively folded κ light chains. This affords the specific targeting and clearance of toxic misfolded κ lights chains while leaving the levels of natively folded κ light chains unperturbed. The antibodies described herein are efficiently taken up by immune cells and enhance phagocytosis and clearance of misfolded κ light chain aggregates, which results in the reduction of κ light chain aggregates in vivo, including amyloidomas that are present in patients that have AL amyloidosis or other plasma cell disorder. The superior binding specificity to toxic κ protein aggregates make the anti-human κ light chain antibodies and their binding fragments especially attractive molecules for detecting misfolded κ light chains and amyloid deposits as well as diagnosing and treating κ light chain-related disorders, such as AL amyloidosis, with minimal off-target effects.

Described herein is an anti-human kappa light chain antibody and human kappa light chain binding fragment thereof comprising: (a) a heavy chain complementarity determining region 1 (H-CDR1) comprising an amino acid sequence set forth in SEQ ID NO: 2; (b) a heavy chain complementarity determining region 2 (H-CDR2) comprising an amino acid sequence set forth in SEQ ID NO: 3; (c) a heavy chain complementarity determining region 3 (H-CDR3) comprising an amino acid sequence set forth in SEQ ID NO: 4; (d) a light chain complementarity determining region 1 (L-CDR1) comprising an amino acid sequence set forth in SEQ ID NO: 6; (e) a light chain complementarity determining region 2 (L-CDR2) comprising an amino acid sequence set forth in SEQ ID NO: 7; and (f) a light chain complementarity determining region 3 (L-CDR3) comprising an amino acid sequence set forth in SEQ ID NO: 8.

In some embodiments, the anti-human kappa light chain antibody and human kappa light chain binding fragment thereof further comprises: (a) a heavy chain variable region comprising an H-CDR1, H-CDR2, and H-CDR3 as described in the amino acid sequences set forth in SEQ ID Nos: 2, 3, and 4, respectively; and (b) a light chain variable region comprising an L-CDR1, L-CDR2, and L-CDR3 as described in the amino acid sequences set forth in SEQ ID Nos: 6, 7, and 8, respectively. In some cases, the heavy chain variable region comprises an amino acid sequence having at least about 80%, 85%, 90%, 95%, 97%, 98%, or 99% sequence identity of SEQ ID NO: 1, and comprises an H-CDR1, H-CDR2, and H-CDR3 as described in the amino acid sequences set forth in SEQ ID Nos: 2, 3, and 4, respectively; and the light chain variable region comprises an amino acid sequence having at least about 80%, 85%, 90%, 95%, 97%, 98%, or 99% sequence identity of SEQ ID NO: 5, and comprises an L-CDR1, L-CDR2, and L-CDR3 as described in the amino acid sequences set forth in SEQ ID Nos: 6, 7, and 8, respectively. In some cases, the heavy chain variable region comprises the amino acid sequence of SEQ ID NO: 1; and the light chain variable region comprises the amino acid sequence of SEQ ID NO: 5. In some embodiments, the heavy chain variable region comprises the amino acid sequence of SEQ ID NO: 1; or the light chain variable region comprises the amino acid sequence of SEQ ID NO: 5.

Also provided herein is an anti-human kappa light chain antibody and human kappa light chain binding fragment thereof comprising: (a) a heavy chain variable region comprising an amino acid sequence of SEQ ID NO: 1; and (b) a light chain variable region comprising an amino acid sequence of SEQ ID NO: 5.

Also provided and described herein is an anti-human kappa light chain antibody and human kappa light chain binding fragment thereof comprising: (a) a heavy chain complementarity determining region 1 (H-CDR1) comprising an amino acid sequence set forth in SEQ ID NO: 10; (b) a heavy chain complementarity determining region 2 (H-CDR2) comprising an amino acid sequence set forth in SEQ ID NO: 11; (c) a heavy chain complementarity determining region 3 (H-CDR3) comprising an amino acid sequence set forth in SEQ ID NO: 12; (d) a light chain complementarity determining region 1 (L-CDR1) comprising an amino acid sequence set forth in SEQ ID NO: 14; (e) a light chain complementarity determining region 2 (L-CDR2) comprising an amino acid sequence set forth in SEQ ID NO: 15; and (f) a light chain complementarity determining region 3 (L-CDR3) comprising an amino acid sequence set forth in SEQ ID NO: 16. In some cases, the anti-human kappa light chain antibody and human kappa light chain binding fragment thereof further comprises: (a) a heavy chain variable region comprising the H-CDR1, H-CDR2, and H-CDR3 as described in the amino acid sequences set forth in SEQ ID Nos: 10, 11, and 12, respectively; and (b) a light chain variable region comprising the L-CDR1, L-CDR2, and L-CDR3 as described in the amino acid sequences set forth in SEQ ID Nos: 14, 15, and 16, respectively. In some cases, the heavy chain variable region comprises an amino acid sequence having at least about 80%, 85%, 90%, 95%, 97%, 98%, or 99% sequence identity of SEQ ID NO: 9, and comprises an H-CDR1, H-CDR2, and H-CDR3 as described in the amino acid sequences set forth in SEQ ID Nos: 10, 11, and 12, respectively; and the light chain variable region comprises an amino acid sequence having at least about 80%, 85%, 90%, 95%, 97%, 98%, or 99% sequence identity of SEQ ID NO: 13, and comprises an L-CDR1, L-CDR2, and L-CDR3 as described in the amino acid sequences set forth in SEQ ID Nos: 14, 15, and 16, respectively. In some cases, the heavy chain variable region comprises the amino acid sequence of SEQ ID NO: 9; and the light chain variable region comprises the amino acid sequence of SEQ ID NO: 13. In some embodiments, the heavy chain variable region comprises the amino acid sequence of SEQ ID NO: 9; or the light chain variable region comprises the amino acid sequence of SEQ ID NO: 13.

Also provided herein is an anti-human kappa light chain antibody and human kappa light chain binding fragment thereof comprising: (a) a heavy chain variable region comprising an amino acid sequence of SEQ ID NO: 9; and (b) a light chain variable region comprising an amino acid sequence of SEQ ID NO: 13.

This disclosure also provides and describes an anti-human kappa light chain antibody and human kappa light chain binding fragment thereof comprising: (a) a heavy chain complementarity determining region 1 (H-CDR1) comprising an amino acid sequence set forth in SEQ ID NO: 18; (b) a heavy chain complementarity determining region 2 (H-CDR2) comprising an amino acid sequence set forth in SEQ ID NO: 19; (c) a heavy chain complementarity determining region 3 (H-CDR3) comprising an amino acid sequence set forth in SEQ ID NO: 20; (d) a light chain complementarity determining region 1 (L-CDR1) comprising an amino acid sequence set forth in SEQ ID NO: 22; (e) a light chain complementarity determining region 2 (L-CDR2) comprising an amino acid sequence set forth in SEQ ID NO: 23; and (f) a light chain complementarity determining region 3 (L-CDR3) comprising an amino acid sequence set forth in SEQ ID NO: 24. In some cases, the anti-human kappa light chain antibody and human kappa light chain binding fragment thereof further comprises: (a) a heavy chain variable region comprising the H-CDR1, H-CDR2, and H-CDR3 as described in the amino acid sequences set forth in SEQ ID Nos: 18, 19, and 20, respectively; and (b) a light chain variable region comprising the L-CDR1, L-CDR2, and L-CDR3 as described in the amino acid sequences set forth in SEQ ID Nos: 22, 23, and 24, respectively. In some cases, the heavy chain variable region comprises an amino acid sequence having at least about 80%, 85%, 90%, 95%, 97%, 98%, or 99% sequence identity of SEQ ID NO: 17, and comprises an H-CDR1, H-CDR2, and H-CDR3 as described in the amino acid sequences set forth in SEQ ID Nos: 18, 19, and 20, respectively; and the light chain variable region comprises an amino acid sequence having at least about 80%, 85%, 90%, 95%, 97%, 98%, or 99% sequence identity of SEQ ID NO: 21, and comprises an L-CDR1, L-CDR2, and L-CDR3 as described in the amino acid sequences set forth in SEQ ID Nos: 22, 23, and 24, respectively. In some cases, the heavy chain variable region comprises the amino acid sequence of SEQ ID NO: 17; and the light chain variable region comprises the amino acid sequence of SEQ ID NO: 21. In some embodiments, the heavy chain variable region comprises the amino acid sequence of SEQ ID NO: 17; or the light chain variable region comprises the amino acid sequence of SEQ ID NO: 21.

Also provided herein is an anti-human kappa light chain antibody and human kappa light chain binding fragment thereof comprising: (a) a heavy chain variable region comprising an amino acid sequence of SEQ ID NO: 17; and (b) a light chain variable region comprising an amino acid sequence of SEQ ID NO: 21.

Another aspect provided and described herein is an anti-human kappa light chain antibody and human kappa light chain binding fragment thereof comprising: (a) a heavy chain complementarity determining region 1 (H-CDR1) comprising an amino acid sequence set forth in SEQ ID NO: 26; (b) a heavy chain complementarity determining region 2 (H-CDR2) comprising an amino acid sequence set forth in SEQ ID NO: 27; (c) a heavy chain complementarity determining region 3 (H-CDR3) comprising an amino acid sequence set forth in SEQ ID NO: 28; (d) a light chain complementarity determining region 1 (L-CDR1) comprising an amino acid sequence set forth in SEQ ID NO: 30; (e)
a light chain complementarity determining region 2 (L-CDR2) comprising an amino acid sequence set forth in SEQ ID NO: 31; and (f) a light chain complementarity determining region 3 (L-CDR3) comprising an amino acid sequence set forth in SEQ ID NO: 32. In some cases, the anti-human kappa light chain antibody or human kappa light chain binding fragment thereof comprises: (a) a heavy chain variable region comprising the H-CDR1, H-CDR2, and H-CDR3 as described in the amino acid sequences set forth in SEQ ID Nos: 26, 27, and 28, respectively; and (b) a light chain variable region comprising the L-CDR1, L-CDR2, and L-CDR3 as described in the amino acid sequences set forth in SEQ ID Nos: 30, 31, and 32, respectively. In some cases, the heavy chain variable region comprises an amino acid sequence having at least about 80%, 85%, 90%, 95%, 97%, 98%, or 99% sequence identity of SEQ ID NO: 25, and comprises an H-CDR1, H-CDR2, and H-CDR3 as described in the amino acid sequences set forth in SEQ ID Nos: 26, 27, and 28, respectively; and the light chain variable region comprises an amino acid sequence having at least about 80%, 85%, 90%, 95%, 97%, 98%, or 99% sequence identity of SEQ ID NO: 29, and comprises an L-CDR1, L-CDR2, and L-CDR3 as described in the amino acid sequences set forth in SEQ ID Nos: 26, 27, and 28, respectively. In some cases, the heavy chain variable region comprises the amino acid sequence of SEQ ID NO: 25; and the light chain variable region comprises the amino acid sequence of SEQ ID NO: 29. In some embodiments, the heavy chain variable region comprises the amino acid sequence of SEQ ID NO: 25; or the light chain variable region comprises the amino acid sequence of SEQ ID NO: 29.

Also provided herein is an anti-human kappa light chain antibody and human kappa light chain binding fragment thereof comprising: (a) a heavy chain variable region comprising an amino acid sequence of SEQ ID NO: 25; and (b) a light chain variable region comprising an amino acid sequence of SEQ ID NO: 29.

In some embodiments, the anti-human κ light chain antibody is a chimeric antibody, a humanized antibody, or a human antibody. In some embodiments, the anti-human κ light chain antibody is a humanized antibody. In some embodiments, the anti-human κ light chain antibody is a human antibody. The anti-human κ light chain antibody can also be a monoclonal antibody (e.g., a monoclonal human antibody or a monoclonal humanized antibody). Alternatively, the anti-human κ light chain antibody can also be a polyclonal antibody (e.g., a polyclonal human antibody or a polyclonal humanized antibody). It is also contemplated that the anti-human κ light chain antibody can, in some embodiments, be a multispecific antibody, such as a bispecific human antibody, a trispecific antibody, etc. In some cases, the human κ light chain binding antibody fragment comprises a Fab, a Fab′, a F(ab′)₂, a scFv, a dsFv, a ds-scFv, a dimer, a minibody, or a diabody. In some cases, the anti-human kappa light chain antibody or human kappa light chain binding fragment thereof is an IgG antibody. In some cases, the IgG antibody is an IgG1 isotype. In some cases, the IgG antibody is an IgG4 isotype. In some cases, the anti-kappa light chain antibody or human kappa light chain binding fragment thereof is coupled to a label. In some cases, the label comprises a chemical label, a biological label, a fluorescent label, a radioisotope label, or any combination thereof. In some cases, the anti-human kappa light chain antibody or human kappa light chain binding fragment thereof binds to a kappa light chain aggregate. In some cases, the anti-human kappa light chain antibody or human kappa light chain binding fragment thereof is characterized by a melting temperature of at least about 70 °C, at least about 80 °C, or at least about 90 °C In some cases, the anti-human kappa light chain antibody or human kappa light chain binding fragment thereof is characterized by a higher binding affinity for a kappa light chain aggregate than a native kappa light chain. In some cases, the anti-human kappa light chain antibody or human kappa light chain binding fragment thereof is characterized by an equilibrium constant (K_{D}) for binding a misfolded κ light chain or kappa light chain aggregate that is no more than about 40 nM, no more than about 30 nM, no more than about 20 nM, no more than about 10 nM, no more than about 5 nM, no more than about 2 nM, no more than about 1 nM, no more than about 0.5 nM, no more than about 0.1 nM, no more than about 0.05 nM, or no more than about 0.01 nM. In some embodiments, the anti-human kappa light chain antibody or human kappa light chain binding fragment thereof is characterized by an equilibrium constant (K_{D}) for binding an kappa light chain aggregate that is at least about 0.01 nM, at least about 0.05 nM, at least about 0.1 nM, at least about 0.5 nM, at least about 1 nM, at least about 5 nM, or at least about 10 nM. In some cases, the K_{D} is measured by an ELISA. In some cases, the K_{D} is measured by a biolayer interferometry sensorgram. In some cases, the anti-human kappa light chain antibody or human kappa light chain binding fragment thereof is capable of being phagocytosed by a THP-1 monocyte cell line.

Also provided and described herein is a pharmaceutical composition comprising any one or more of the anti-human kappa light chain antibodies or human kappa light chain binding fragments thereof provided herein and a pharmaceutically acceptable excipient, diluent, or carrier. In some cases, the pharmaceutical composition is formulated for intravenous administration. In some embodiments, the pharmaceutical composition is for use in the treatment of a disease or condition characterized by kappa light chain aggregates in the subject. In some embodiments, the disease or condition characterized by kappa light chain aggregates is a plasma cell disorder, systemic amyloidosis, or light chain amyloidosis. In some embodiments, the plasma cell disorder is a plasma cell dyscrasia. In some embodiments, the plasma cell dyscrasia is amyloidosis, monoclonal gammopathy of unknown significance (MGUS), multiple myeloma (MM), plasma cell leukemia (PCL), or any combination thereof. In some embodiments, the plasma cell dyscrasia is systemic amyloidosis. In some embodiments, the plasma cell dyscrasia is light chain amyloidosis. In some embodiments, the plasma cell dyscrasia is cardiac amyloidosis. In some embodiments, the plasma cell dyscrasia is renal amyloidosis.

Also provided herein is a nucleic acid or a plurality of nucleic acids encoding any one or more of the anti-human kappa light chain antibodies or human kappa light chain binding fragments thereof provided herein.

Also disclosed herein is a cell comprising a nucleic acid or a plurality of nucleic acids encoding any one or more of the anti-human kappa light chain antibodies or human kappa light chain binding fragments thereof of this disclosure. In some cases, the cell is a eukaryotic cell. In some cases, the eukaryotic cell is a Chines Hamster Ovary (CHO) cell. In some cases, the cell is a prokaryotic cell.

Also provided and described herein is a method of diagnosing amyloidosis in a sample, the method comprising contacting a sample with any one or more of the anti-human kappa light chain antibodies or human kappa light chain binding fragments thereof provided herein.

In yet another aspect, this disclosure provides a method of detecting a kappa light chain aggregate in a sample, the method comprising contacting the sample with any one or more of the anti-human kappa light chain antibodies or human kappa light chain binding fragments thereof provided herein.

In some cases, the sample comprises blood, serum, plasma, solid tissue, liquid biopsy, bone marrow, cerebrospinal fluid, or any combination thereof. In some embodiments, the sample comprises a cardiac tissue, a kidney tissue. a pulmonary tissue, an intestinal tissue, a gastrointestinal (GI) tissue, a skin tissue, a subcutis tissue, a soft tissue, a tendon tissue, a bone tissue, a synovium, a gall bladder tissue, a pharyngeal tissue, a spleen tissue, a lymph node tissue, a neural tissue, or an immune tissue. In some cases, the sample is obtained from a subject. In some cases, the subject is a human.

Also provided and described herein is a method of increasing clearance of a kappa light chain aggregate, the method comprising contacting the kappa light chain aggregate with any one or more of the anti-human kappa light chain antibodies or human kappa light chain binding fragments thereof provided herein. In some embodiments, the method results in a decrease in the level of the kappa light chain aggregate in a cardiac tissue, a renal tissue, a pulmonary tissue, an intestinal tissue, a gastrointestinal (GI) tissue, a soft tissue, a synovial tissue, a spleen tissue, or an immune tissue compared to a level of the kappa light chain aggregate in a similar tissue prior to the contacting. In some embodiments, the method results in a decrease in the level of the kappa light chain aggregate in a cardiac tissue compared to a level of the kappa light chain aggregate in a similar cardiac tissue prior to the contacting. In some embodiments, the method results in a decrease in the level of the kappa light chain aggregate in a similar renal tissue compared to a level of the kappa light chain aggregate in a similar renal tissue prior to the contacting. In some embodiments, the method results in a decrease in the level of the kappa light chain aggregate in a colon tissue compared to a level of the kappa light chain aggregate in a colon tissue prior to the contacting.

Also described herein is a method of reducing kappa light chain aggregates in a subject in need thereof, the method comprising administering the anti-human kappa light chain antibody or human kappa light chain binding fragment thereof of any one or more of the anti-human kappa light chain antibodies or human kappa light chain binding fragments thereof provided herein. In some embodiments, the method results in a decrease in the level or the number of the kappa light chain aggregate in a cardiac tissue, a renal tissue, a pulmonary tissue, an intestinal tissue, a gastrointestinal (GI) tissue, a soft tissue, a synovial tissue, a spleen tissue, or an immune tissue compared to a level or a number or a number of the kappa light chain aggregate in a similar tissue prior to the administering, In some embodiments, the method results in a decrease in the level or the number of the kappa light chain aggregate in a cardiac tissue compared to a level or a number of the kappa light chain aggregate in a similar cardiac tissue prior to the administering. In some embodiments, the method results in a decrease in the level or the number of the kappa light chain aggregate in a renal tissue compared to a level or a number of the kappa light chain aggregate in a similar renal tissue prior to the administering. In some embodiments, the method results in a decrease in the level or the number of the kappa light chain aggregate in a similar colon tissue compared to a level or a number of the kappa light chain aggregate in a similar colon tissue prior to the administering.

This disclosure further provides a method of treating a disease or condition characterized by kappa light chain aggregates in a subject in need thereof, the method comprising administering any one or more of the anti-human kappa light chain antibodies or human kappa light chain binding fragments thereof provided herein. In some cases, the disease or condition characterized by kappa light chain aggregates is a plasma cell disorder. In some cases, the plasma cell disorder comprises a plasma cell dyscrasias. In some cases, the plasma cell dyscrasias comprises an amyloidosis, a monoclonal gammopathy of unknown significance (MGUS), multiple myeloma (MM), plasma cell leukemia (PCL), or any combination thereof. In some cases, the disease or condition characterized by kappa light chain aggregates is systemic amyloidosis. In some cases, the disease or condition characterized by kappa light chain aggregates is light chain amyloidosis. In some embodiments, wherein the disease or condition characterized by kappa light chain aggregates is a cardiac amyloidosis. In some embodiments, the disease or condition characterized by kappa light chain aggregates is a renal amyloidosis. In some cases, the administering any one or more of the anti-human kappa light chain antibodies or human kappa light chain binding fragments thereof provided herein to the subject results in a reduction of kappa light chain aggregates in the subject. In some cases, the administering any one or more of the anti-human kappa light chain antibodies or human kappa light chain binding fragments thereof provided herein to the subject increases a number of macrophages or monocytes surrounding or within an amyloidoma in the subject. In some cases, the administering the anti-human kappa light chain antibody or human kappa light chain binding fragment thereof increases a number of macrophages surrounding or within an amyloidoma in the subject. In some cases, the macrophage is a F4/80+ macrophage. In some cases, the administering the anti-human kappa light chain antibody or human kappa light chain binding fragment thereof increases phagocytic uptake of kappa light chain aggregates. In some cases, the administering the anti-human kappa light chain antibody or human kappa light chain binding fragment thereof decreases a size of an amyloidoma in the subject. In some embodiments, the method results in a decrease in the size of the kappa light chain aggregate in a cardiac tissue, a renal tissue, a pulmonary tissue, an intestinal tissue, a gastrointestinal (GI) tissue, a soft tissue, a synovial tissue, a spleen tissue, or an immune tissue compared to a size of the kappa light chain aggregate in a similar tissue prior to the administering. In some embodiments, the method results in a decrease in the size of the kappa light chain aggregate in a cardiac tissue compared to a size of the kappa light chain aggregate in a similar cardiac tissue prior to the administering. In some embodiments, the method results in a decrease in the size of the kappa light chain aggregate in a renal tissue compared to a size of the kappa light chain aggregate in a similar renal tissue prior to the administering. In some embodiments, the method results in a decrease in the size of the kappa light chain aggregate in a similar colon tissue compared to a size of the kappa light chain aggregate in a similar colon tissue prior to the administering.

Also described and provided herein is a method of manufacturing any one or more of the anti-human kappa light chain antibodies or human kappa light chain binding fragments thereof provided herein, the method comprising: (a) culturing a cell comprising a nucleic acid encoding the anti-human kappa light chain antibody or human kappa light chain binding fragment thereof under conditions sufficient to allow production and secretion of the anti-human kappa light chain antibody or the human kappa light chain binding fragment thereof from the cell; and (b) isolating the anti-human kappa light chain antibody or the human kappa light chain binding fragment thereof from the cell or a supernatant of the cell. In some cases, the cell is a eukaryotic cell. In some cases, the eukaryotic cell comprises a Chinese Hamster Ovary (CHO) cell. In some embodiments, the anti-human kappa light chain antibody or human kappa light chain binding fragment thereof is produced by the method provided and disclosed herein.

In yet another aspect, disclosed herein is a vector comprising: (a) a first nucleic acid sequence encoding a heavy chain variable region comprising an amino acid sequence as set forth in SEQ ID NO: 1, or encoding a heavy chain variable region comprising an H-CDR1, a H-CDR2, and a H-CDR3 as set forth in the amino acid sequence of SEQ ID NO: 1, and/or a second nucleic acid encoding a light chain variable region comprising an amino acid sequence as set forth in SEQ ID NO: 5, or encoding a light chain variable region comprising an L-CDR1, an L-CDR2, and an L-CDR3 as set forth in the amino acid sequence of SEQ ID NO: 5; (b) a first nucleic acid sequence encoding a heavy chain variable region comprising an amino acid sequence as set forth in SEQ ID NO: 9, or encoding a heavy chain variable region comprising an H-CDR1, a H-CDR2, and a H-CDR3 as set forth in the amino acid sequence of SEQ ID NO: 9, and/or a second nucleic acid encoding a light chain variable region comprising an amino acid sequence as set forth in SEQ ID NO: 13, or encoding a light chain variable region comprising an L-CDR1, an L-CDR2, and an L-CDR3 as set forth in the amino acid sequence of SEQ ID NO: 13; (c) a first nucleic acid sequence encoding a heavy chain variable region comprising an amino acid sequence as set forth in SEQ ID NO: 17, or encoding a heavy chain variable region comprising an H-CDR1, a H-CDR2, and a H-CDR3 as set forth in the amino acid sequence of SEQ ID NO: 17, and/or a second nucleic acid encoding a light chain variable region comprising an amino acid sequence as set forth in SEQ ID NO: 21, or encoding a light chain variable region comprising an L-CDR1, an L-CDR2, and an L-CDR3 as set forth in the amino acid sequence of SEQ ID NO: 21; or (d) a first nucleic acid sequence encoding a heavy chain variable region comprising an amino acid sequence as set forth in SEQ ID NO: 25, or encoding a heavy chain variable region comprising an H-CDR1, a H-CDR2, and a H-CDR3 as set forth in the amino acid sequence of SEQ ID NO: 25, and/or a second nucleic acid encoding a light chain variable region comprising an amino acid sequence as set forth in SEQ ID NO: 29, or encoding a light chain variable region comprising an L-CDR1, an L-CDR2, and an L-CDR3 as set forth in the amino acid sequence of SEQ ID NO: 29.

### BRIEF DESCRIPTION OF THE DRAWINGS

The novel features described in this disclosure are set forth with particularity in the appended claims. A better understanding of the features and advantages described herein will be obtained by reference to the following detailed description that sets forth illustrative embodiments, in which the principles of the invention are utilized, and the accompanying drawings of which:

FIG. 1A shows quantification of the thermal stability of four human or humanized monoclonal antibodies described herein expressed as the ratio of fluorescence intensity measured at 350 nm/330 nm. Concentrations of each monoclonal antibody were adjusted to 1 mg/mL, and fluorescence was measured using Nanotemper Tycho. Points on curves indicate inflection points (i.e., melting temperature, Tm)

FIG. 1B depicts quantified melting temperature of four human or humanized monoclonal antibodies corresponding to their respective inflection points depicted in FIG. 1A.

FIG. 2 depicts line graphs that illustrate binding affinity and specificity of four monoclonal human or humanized antibodies described herein to native, alkylated, and aggregated kappa light chain variants measured by antibody capture ELISA. Quantification of binding affinity and specificity is expressed as optical density (absorbance measured at a wavelength of 450 nanometers) versus log base-10 concentration of each human monoclonal antibody. Monoclonal antibodies (20 ng) were captured upright on-plate with anti-IgG Fc antibodies. Biotinylated light chains (LCs) were added at varying concentrations for 1 hour. Neutravidin-HRP was added at 1:20,000 dilution for 30 minutes. Colour was developed with TMB substrate for 150-180 sec. AL09, AL12, and AL103 represent samples of light chains whose sequences are obtained from individual light chain amyloidosis (AL) patients. Equilibrium constants (K_{D}, in nM) obtained from capture ELISAs in FIG. 2 are shown in TABLE 2.

FIGS. 3A-3I show quantification of binding affinity and specificity of four monoclonal human or humanized antibodies described herein to native, alkylated, and aggregated kappa light chain variants measured by competition ELISA: AB1 (FIG. 3A), AB4 (FIG. 3B), AB2 (FIG. 3C), and AB3 (FIG. 3D). Quantification of binding of to native, alkylated, and aggregated kappa light chain variants in the extended competition ELISA experiment is shown for AB1 (FIG. 3E), AB4 (FIG. 3F), AB2 (FIG. 3G), vrAB1 (FIG. 3H), and vrAB2 (FIG. 3I) antibodies. Monoclonal antibodies (20 ng) were captured upright on-plate with anti-IgG Fc antibodies. In a separate plate, biotinylated analyte (2 nM aggregated kappa light chain from AL09) was mixed with serially diluted competitor (aggregated kappa light chains from AL09 patient sample or pooled IgK LCs). The mixed solution was added to candidate monoclonal antibody plate and incubated for 2 hours at room temperature. Neutravidin-horseradish peroxidase (HRP) was added at 1:15,000 dilution for 30 minutes. Colour was developed with 3,3′,5,5′-Tetramethylbenzidine (TMB) substrate for 210 sec. Y-axis is normalized to wells without competitor, which represents 100% analyte still bound.

FIGS. 3J-3N show binding curves of AB1 (FIG. 3J), AB4 (FIG. 3K), AB2 (FIG. 3L), vrAB1 (FIG. 3M), and vrAB2 (FIG. 3N) antibodies to either misfolded or fibrillar κLCs measured by capture ELISA.

FIG. 3O shows quantification of avidity of AB1, AB2, AB4, vrAB1, and vrAB2 antibodies to 20 μM adsorbed and dried AL09 misfolded κLCs measured by indirect ELISA.

FIGS. 4A-4B depict quantification of human monoclonal antibody AB1 binding affinity and specificity to alkylated kappa light chains of AL09 patient sample at 300, 100, 33, 11, 3.7 nM (FIG. 4A) and native kappa light chains of AL09 patient sample at 300 nM and 100 nM (FIG. 4B) as measured by biolayer interferometry (BLI) sensorgrams. Association time, 120 seconds. Dissociation time 600 seconds. Each binding curve was line fitted.

FIGS. 5A-5B depict quantification of humanized monoclonal antibody AB4 binding affinity and specificity to alkylated kappa light chains of AL09 patient sample at 300, 100, 33, 11, 3.7 nM (FIG. 5A) and native kappa light chains of AL09 patient sample at 300 nM and 100 nM (FIG. 5B) as measured by biolayer interferometry (BLI) sensorgrams. Association time, 120 seconds. Dissociation time 600 seconds. Each binding curve was line fitted.

FIGS. 6A-6B depict quantification of humanized monoclonal antibody AB2 binding affinity and specificity to alkylated kappa light chains of AL09 patient sample at 300, 100, 33, 11, 3.7 nM (FIG. 6A) and native kappa light chains of AL09 patient sample at 300 nM and 100 nM (FIG. 6B) as measured by biolayer interferometry (BLI) sensorgrams. Association time, 120 seconds. Dissociation time 600 seconds. Each binding curve was line fitted.

FIGS. 7A-7B depict quantification of humanized monoclonal antibody AB3 to alkylated kappa light chains of AL09 patient sample at 300, 100, 33, 11, 3.7 nM (FIG. 7A) and native kappa light chains of AL09 patient sample at 300 nM and 100 nM (FIG. 7B) as measured by BLI sensorgrams. Association time, 120 seconds. Dissociation time 600 seconds. Each binding curve was line fitted.

FIG. 8A shows quantification of phagocytic uptake kinetics of AB1, AB2, AB3, AB4, or Fc null (AB4) monoclonal antibodies in THP-1 cells following a 2-hour pre-incubation with a mixture of misfolded pHrodo (pH sensitive dye) labelled AL-09 aggregates (160 nM) and increasing concentrations of said monoclonal antibodies. Fluorescence imaging was performed using Cytation 5 (Agilent Systems) and total fluorescence per cell in each well is plotted. EC₅₀ values shown in TABLE 4 were calculated by curve fitting phagocytic uptake kinetics of FIG. 8A.

FIG. 8B shows quantification of phagocytic uptake kinetics of AB1, AB2, AB3, AB4, or Fc null (AB4) monoclonal antibodies in THP-1 cells following a 24-hour pre-incubation with a mixture of misfolded pHrodo (pH sensitive dye) labelled AL-09 aggregates (160 nM) and increasing concentrations of said monoclonal antibodies. Fluorescence imaging was performed using Cytation 5 (Agilent Systems) and total fluorescence per cell in each well is plotted. EC₅₀ values shown in TABLE 4 were calculated by curve fitting phagocytic uptake kinetics of FIG. 8B.

FIGS. 8C-8G show concentration-dependent curves of the percentage of THP1 with uptake of pHrodo-labeled AL09, AL12, AL103, LEN, and SMA κLC fibrils following treatment with AB2 (FIG. 8C), AB1 (FIG. 8D), AB4 (FIG. 8E), vrAB1 (FIG. 8F), or vrAB2 antibodies (FIG. 8G).

FIGS. 8H-8L depict EC₅₀ values for uptake of pHrodo-labeled AL09, AL12, AL103, LEN, and SMA κLC fibrils following treatment with AB2 (FIG. 8H), AB1 (FIG. 8I), AB4 (FIG. 8J), vrAB1 (FIG. 8K), or vrAB2 antibodies (FIG. 8L) calculated from the concentration curves in FIGS. 8C-8G.

FIGS. 9A-9C depict colocalization of amyloidoma and monoclonal antibody AB4. FIG. 9A shows the in vivo fluorescent signal of VivoTag 800 dye bound to amyloidoma in a test mouse (t) subcutaneously injected with amyloid protein (0.5 mg) with matrigel compared to A control mouse (c) that was subcutaneously injected with Matrigel alone. Both test (t) and control (c) mice received a 10 mg/kg dose of fluorescently labelled test antibody AB4. FIG. 9B shows the in vivo fluorescent signal of Alexa 680-labelled AB4 monoclonal antibody in test and control mice. FIG. 9C shows co-localization of the Alexa 680-labelled AB4 monoclonal antibody with the Vivotag 800-labelled amyloidoma in both test and control mice.

FIGS. 10A-10C depict immunohistological images of formalin-fixed, paraffin-embedded, and subcutaneously implanted amyloid aggregates (0.5 mg) that were excised from mice and stained with biotinylated goat anti-human IgG to detect presence of human IgG in the amyloid after tail vein infusion of AB4 antibody or isotype human IgG control. FIG. 10A shows a lack of colocalization of the human isotype control antibody with amyloid aggregates (darkened punctate signal) in tissue obtained from a mouse treated with 10 mg/kg of isotype control antibody. FIG. 10B shows colocalization of the AB4 monoclonal antibody with amyloid aggregates in tissue obtained from a mouse treated with 10 mg/kg of the AB4 monoclonal antibody. Arrows show representative positive colocalization spots (heavy dark puncta) of the human IgG AB4 antibody with the amyloid aggregates. FIG. 10C shows representative immunohistological images of VivoTag800-AL09 fibril fluorescence and AB2, AB4, and isotype antibody horseradish peroxidase (HRP) staining in amyloidomas excised from treated mice. Amyloidomas excised from untreated mice were also stained for VivoTag800-AL09 and HRP signal. Images show specific targeting of fibrils by LX-96 antibodies compared to untreated and isotype controls. Scale bar=100 microns.

FIGS. 11A-11B depict immunohistological images of formalin-fixed, paraffin-embedded, and subcutaneously implanted amyloid aggregates (0.5 mg) that were excised from mice 4 days after tail vein infusion of isotype or AB4 monoclonal antibody and stained with anti-F4/80 antibody, a macrophage-specific marker. F4/80+ cells (dark signal) and surround a central mass (amyloidoma). FIG. 11A shows a representative image of tissue obtained from a mouse treated with 10 mg/kg isotype control IgG antibody. Note diffuse F4/80+ signal throughout the tissue section. FIG. 11B shows a representative image of tissue obtained from a mouse treated with 10 mg/kg of AB4 that exhibits robust localization of F4/80+ macrophages (dark spots) to the amyloidoma compared to the isotype matched control in FIG. 11A. Scale bar=500 microns.

FIG. 12 depicts quantification of the percent change in pHrodo-labelled amyloid signal in macrophages within amyloidomas of mice treated with either AB1 antibody, isotype control antibody, or amyloid alone. Data indicate increased phagocytic uptake of amyloid in mice treated with the AB1 antibody compared to the isotype control or amyloid alone.

FIGS. 13A-13B depict serum concentration-time curves of human IgG levels in mice treated with 2 mg/kg, 10 mg/kg, or 30 mg/kg doses of human IgG1 isotype control (FIG. 13A) or AB4 test antibody (FIG. 13B). All mice received a subcutaneous injection of 0.5 mg of amyloidoma. Samples were analyzed for human IgG levels using a Meso scale discovery (MSD) based ELISA platform.

FIG. 14A shows representative longitudinal fluorescent images of VivoTag-labelled amyloidoma in untreated mice (no antibody administered) or in mice that received a 30 mg/kg tail vein injection of either AB1, AB2, or AB4 human or humanized monoclonal antibodies monitored at 0, 24, 48, 72, and 96 hours. All mice were subcutaneously implanted with AL09 amyloidoma (0.5 mg) on Day 0. Each animal received a single intravenous administration of AB1, AB2, or AB4 human or humanized monoclonal antibodies 6 hours following implantation of AL09 amyloidoma. Three mice were imaged per condition.

FIG. 14B depicts quantification of VivoTag fluorescence measured within in vivo images of FIG. 14A untreated mice (No Ab group) or mice treated with either AB1 or AB4 human or humanized monoclonal antibodies at 0, 24, 48, 72, and 96 hours. Total VivoTag fluorescence was measured and normalized to baseline fluorescence (0 hours). After an initial 24-hour wash-out period, the amyloidoma stabilized in the untreated animals but significantly reduced in size in the animals treated with either AB1 or AB4 antibodies. Error bars indicate standard deviation.

FIG. 14C depicts quantification of VivoTag-AL09 fibril fluorescence measured within in vivo images obtained from untreated mice (No Ab group) or mice treated with AB2 human or humanized monoclonal antibodies at 0, 24, 48, 72, and 96 hours post-injection. Prior to AB2 treatment, all mice received a subcutaneous injection of AL09 amyloidoma (0.5 mg) on Day 0. Total VivoTag fluorescence was measured and normalized to baseline fluorescence (0 hours). After an initial 24-hour wash-out period, the amyloidoma stabilized in the untreated animals but significantly reduced in size in the animals treated with either AB1 or AB4 antibodies. Error bars indicate standard deviation.

FIG. 14D depicts quantification of amyloid size change measured by VivoTag-AL09 fibril fluorescence in vivo in vehicle-treated mice or mice treated with AB2, AB4, vrAB1, vrAB2, or isotype antibodies at 0, 24, 48, 72, 96, 120, 144, 168, 192, and 216 hours post-injection. Dose for all antibody treatment groups: 30 mg/kg.

FIG. 14E shows quantification of the average half-life of kappa light chain amyloid in vehicle-treated mice or mice treated with AB2, AB4, vrAB1, vrAB2, or isotype antibodies based on the amyloid decay curves shown in FIG. 14D.

FIG. 14F shows quantification of the average decay rate constant of kappa light chain amyloid in vehicle-treated mice or mice treated with AB2, AB4, vrAB1, vrAB2, or isotype antibodies based on the amyloid decay curves shown in FIG. 14D.

FIG. 15A shows representative images of amyloidomas excised from untreated mice or mice treated with isotype human IgG control, AB1, or AB4 antibodies 96 hours after intravenous injection of 30 mg/kg of respective isotype or antibody.

FIG. 15B shows quantification of the average area (mm²) of amyloidomas excised from untreated mice (No Ab group) or mice treated with either AB1 or AB4 human or humanized monoclonal antibodies 96 hours after intravenous injection of antibodies. The areas of the amyloidoma were calculated based on the boundaries drawn in FIG. 15A. While isotype-treated animals showed reduction in amyloidoma size, it was not significant compared to the untreated animals. Mice treated with either AB1 or AB4 showed a significant reduction in amyloidoma area compared to the untreated (No Ab) group or the isotype control group.

FIG. 16 shows representative longitudinal fluorescent images of VivoTag-labelled amyloidoma in three untreated mice (no antibody administered) at 0, 24, 48, 72, and 96 hours following injection of subcutaneously implanted with AL09 amyloidoma (0.5 mg). Insets on right: Representative images of amyloidomas excised from Mouse 1, Mouse 2, or Mouse 3 after 96 hours. Gray boundaries indicate the perimeter of the amyloidoma within the region of interest (ROI).

FIG. 17 shows representative longitudinal fluorescent images of VivoTag-labelled amyloidoma in three mice treated with AB4 human humanized monoclonal antibody at 0, 24, 48, 72, and 96 hours following a single intravenous injection of 30 mg/kg of AB4 antibody. Mice received a subcutaneous injection with AL09 amyloidoma (0.5 mg) 6 hours before intravenous antibody injections. Insets on right: Representative images of amyloidomas excised from Mouse 1, Mouse 2, or Mouse 3 after 96 hours. Gray boundaries indicate the perimeter of the amyloidoma within the region of interest (ROI).

FIG. 18 shows representative longitudinal fluorescent images of VivoTag-labelled amyloidoma in three mice treated with AB1 human monoclonal antibody at 0, 24, 48, 72, and 96 hours following a single intravenous injection of 30 mg/kg of ABI antibody. Mice received a subcutaneous injection with AL09 amyloidoma (0.5 mg) 6 hours before intravenous antibody injections. Insets on right: Representative images of amyloidomas excised from Mouse 1, Mouse 2, or Mouse 3 after 96 hours. Gray boundaries indicate the perimeter of the amyloidoma within the region of interest (ROI).

FIGS. 19A-19F depict quantification of the average binding affinities (K_{D}, nM) to misfolded KLCs in IGVK1 and IGVK4 patient samples by constant region-targeting anti-KLC antibodies AB4 (FIG. 19A), AB1 (FIG. 19B), AB3 FIG. (19C), and AB2 FIG. (19D) and variable region-targeting anti-KLC antibodies vrAB1 (FIG. 19E) and vrAB2 (FIG. 19F), as measured by BLI sensorgrams. *K_{D}>300 nM. Alkylated kappa light chain sequences obtained from patients LEN and SMA have light chains with IGKV4 v-gene usage. Alkylated kappa light chain sequences obtained from patients AL09, AL12, and AL103 have light chains derived with IGKV1 v-gene usage.

FIGS. 20A-20C show quantified amyloid reduction curves obtained from in vivo VivoTag800-AL09 fibril fluorescence following treatment with a of either isotype, AB4, or AB2 at 0.3 mg/kg (FIG. 20A), 1 mg/kg (FIG. 20B), or 3 mg/kg (FIG. 20C) administered to AL09 misfolded κ light chain-implanted mice from Day 0 to Day 7 post-treatment. Data are normalized to VivoTag800-AL09 fibril fluorescence measured at Day 0.

FIG. 20D depicts quantification of the average decay rate constant calculated from the amyloid reduction curves of FIGS. 20A-20C.

FIG. 20E depicts quantification of the average half-life calculated from the amyloid reduction curves of FIGS. 20A-20C.

FIG. 20F depicts quantification of the average time to 50% κ light chain aggregate reduction calculated from the VivoTag800-AL09 fibril fluorescence measured in FIGS. 20A-20C.

FIG. 21A depicts quantification of the average time course for 50% κ light chain aggregate reduction in animals treated with 3, 10, or 30 mg/kg of AB1 or AB2 compared to the untreated group based on the VivoTag800-AL09 fibril fluorescence measured from day 0-4.

FIG. 21B depicts quantification of the average time course for 50% κ light chain aggregate reduction in animals treated with 3, 10, or 30 mg/kg of AB4-treated, untreated and isotype-treated groups based on the VivoTag800-AL09 fibril fluorescence measured from day 0-4.

FIG. 21C depicts quantification of the average time course for 50% κ light chain aggregate reduction in animals treated with 3, 10, or 30 mg/kg of AB4 or isotype.

FIG. 21D depicts quantification of the average time course for 50% κ light chain aggregate reduction in animals treated with 3, 10, or 30 mg/kg of AB1 or AB2.

FIG. 21E depicts quantification of the average time course for 50% κ light chain aggregate reduction in animals treated with 0.3, 1, 3, 10, or 30 mg/kg intravenous injection of AB4, AB2, or isotype control antibodies.

FIG. 22 depicts representative images of immunohistochemical (IHC) staining of misfolded κ light chains with chimerized AB2 (middle column) and AB4 (Left column) anti-κ light chain antibodies in formalin-fixed, paraffin-embedded renal glomerular tissue (Top row) and cardiac muscle fibers (Bottom Row). Staining signal is 3,3′-diaminobenzidine (DAB) for colorimetric detection of HRP (brown). Tissue was counterstained with hematoxylin (blue). IHC staining colocalizes with fluorescent amyloid-specific dye, Congo Red (Right column).

FIG. 23 shows representative images of immunohistochemical (IHC) staining of misfolded κ light chains with rabbit-chimerized AB2 and AB4 anti-κ light chain antibodies, rabbit-chimerized variable region-targeting anti-κ light chain antibodies vrAB1 and vrAB2 in formalin-fixed, paraffin-embedded tissue sections of kidney hilus. Positive control: phospho-specific anti-kappa light chain antibody (anti-κ pAb).

FIG. 24 shows representative images of immunohistochemical (IHC) staining of misfolded κ light chains with rabbit-chimerized AB2 and AB4 anti-κ light chain antibodies, rabbit-chimerized variable region-targeting anti-κ light chain antibodies vrAB1 and vrAB2 in formalin-fixed, paraffin-embedded colon tissue sections. Positive control: anti-κ pAb.

FIG. 25 shows representative images of immunohistochemical (IHC) staining of misfolded κ light chains with rabbit-chimerized AB2 and AB4 anti-κ light chain antibodies, and rabbit-chimerized variable region-targeting anti-κ light chain antibodies vrAB1 and vrAB2 in formalin-fixed, paraffin-embedded synovium sections.

FIG. 26 shows representative images of immunohistochemical (IHC) staining of misfolded κ light chains with rabbit-chimerized AB2 and AB4 anti-κ light chain antibodies, rabbit-chimerized variable region-targeting anti-κ light chain antibodies vrAB1 and vrAB2 in formalin-fixed, paraffin-embedded heart tissue sections. Positive control: anti-κ pAb. Scale bar=100 micron.

FIG. 27 depicts representative images of immunohistochemical (IHC) staining of misfolded κ light chains with rabbit-chimerized AB2 and AB4 anti-κ light chain antibodies, rabbit-chimerized variable region-targeting anti-κ light chain antibodies vrAB1 and vrAB2 in formalin-fixed, paraffin-embedded kidney (hilus) tissue sections. Positive control: anti-κ pAb. Scale bar=100 micron.

FIG. 28 depicts representative images of immunohistochemical (IHC) staining of misfolded κ light chains with rabbit-chimerized AB2 and AB4 anti-κ light chain antibodies, rabbit-chimerized variable region-targeting anti-κ light chain antibodies vrAB1 and vrAB2 in formalin-fixed, paraffin-embedded kidney (glomerulus) tissue sections. Scale bar=100 micron.

FIG. 29 depicts representative images of immunohistochemical (IHC) staining of misfolded κ light chains with rabbit-chimerized AB2 and AB4 anti-κ light chain antibodies, rabbit-chimerized variable region-targeting anti-κ light chain antibodies vrAB1 and vrAB2 in formalin-fixed, paraffin-embedded kidney (tubule) tissue sections. Scale bar=100 micron.

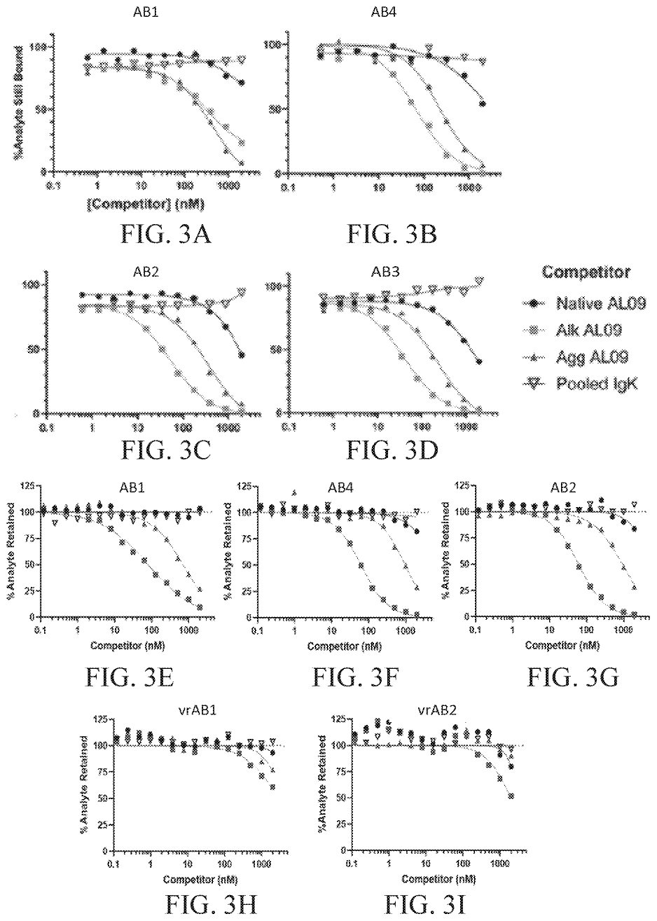

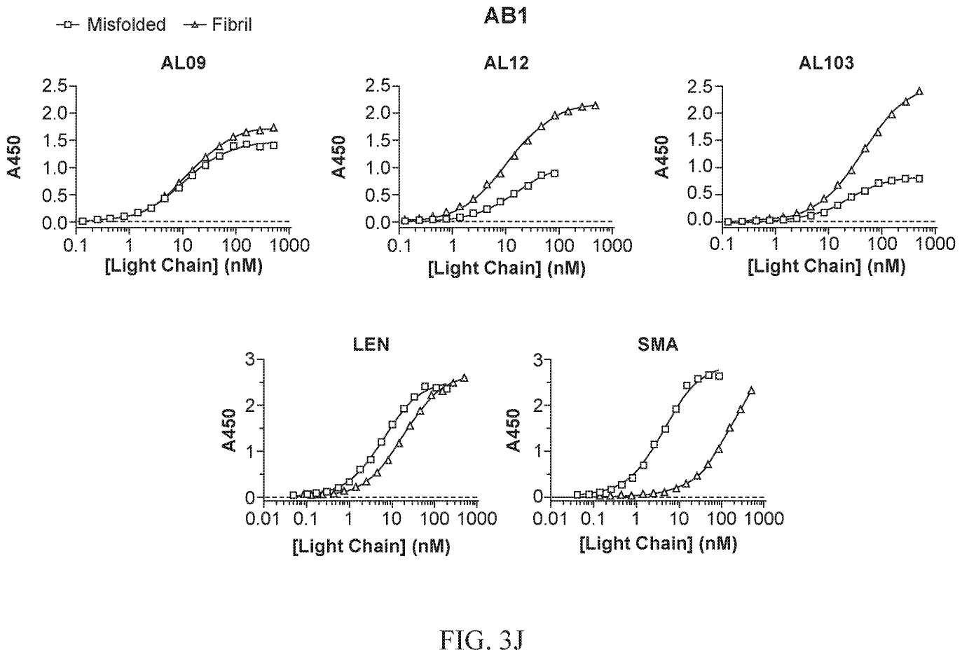

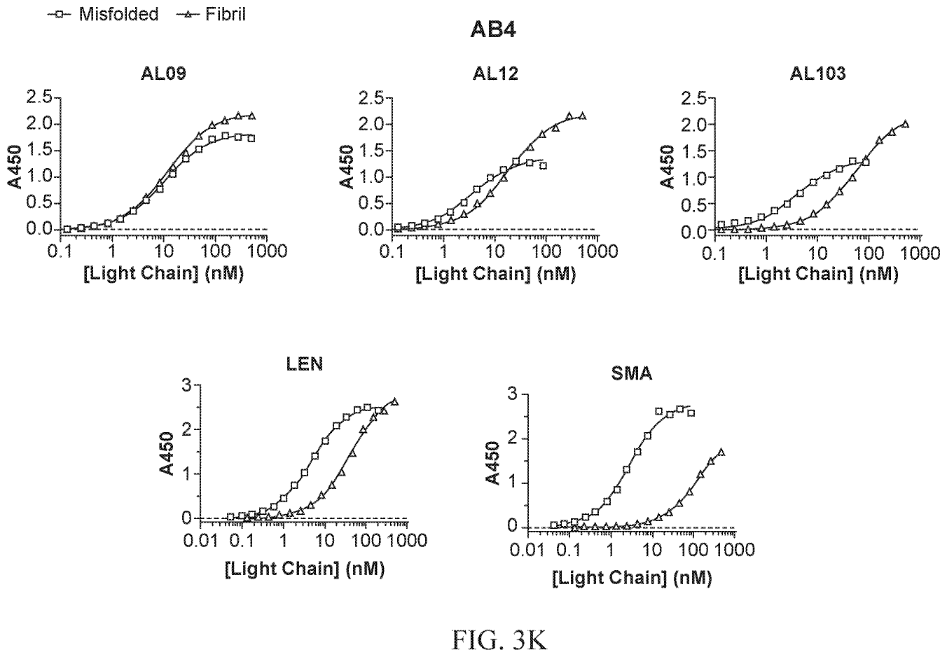

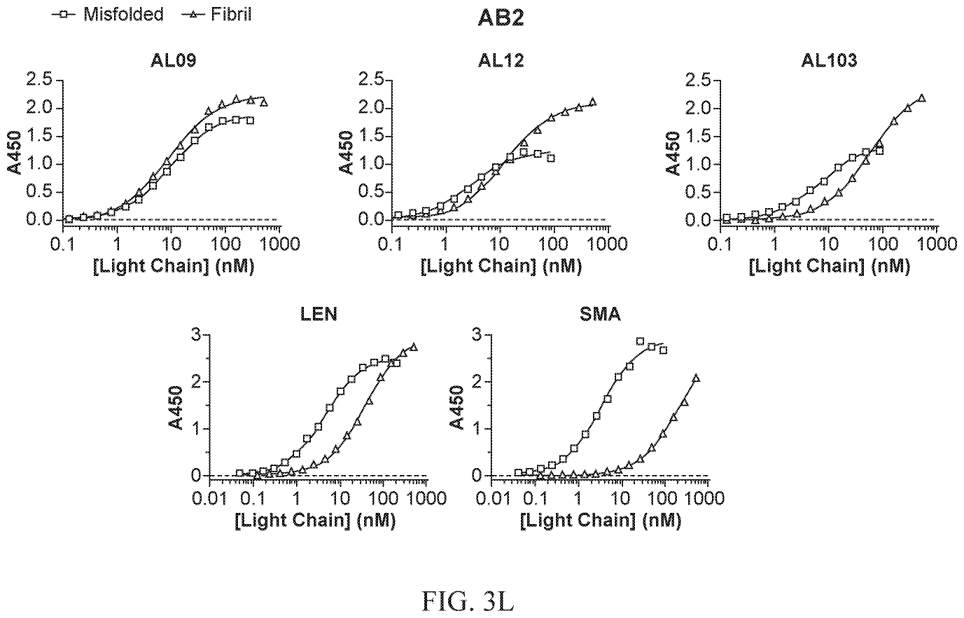

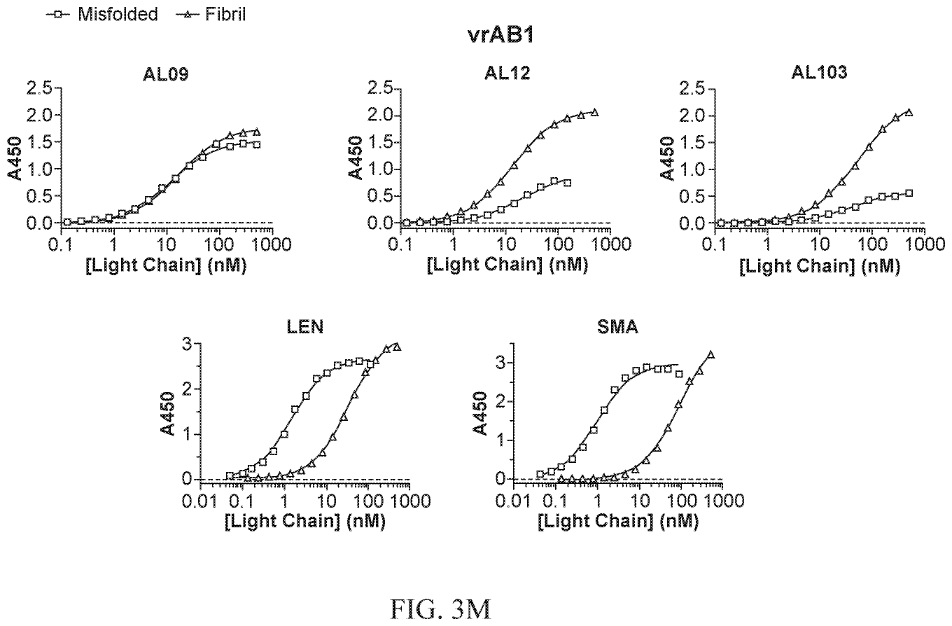

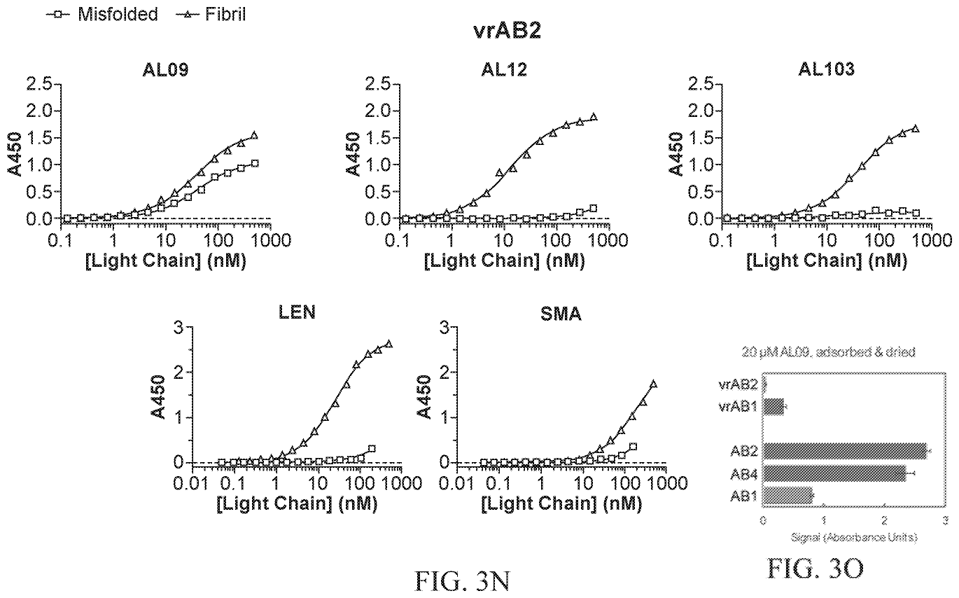

FIG. 3O depicts representative images of immunohistochemical (IHC) staining of misfolded κ light chains with rabbit-chimerized AB2 and AB4 anti-κ light chain antibodies, rabbit-chimerized variable region-targeting anti-κ light chain antibodies vrAB1 and vrAB2, and phospho-specific anti-kappa light chain antibody (positive control) in formalin-fixed, paraffin-embedded bone marrow tissue sections. Scale bar=50 micron.

FIGS. 31A-31C show binding of AB4 (FIG. 3IA), AB2 (FIG. 3IB), and isotype (FIG. 3IC) antibodies to non-AL human, swine, canine and human AL09 (AL amyloidosis patient sample; positive control) serum proteins by indirect ELISA. Plates were coated overnight with human, swine, or canine serum at the indicated dilutions in PBS. 100 ng of misfolded AL09 was included as a positive control. Plates were blocked with BSA, and probed with biotinylated AB4, AB2, or a non-binding isotype control antibody. Bound biotinylated antibody was detected using Neutravidin-HRP and TMB substrate. Background signal was measured in reference wells (no primary antibody) and subtracted from each of the corresponding antibody signals. Y-axis plots the increase in absorbance at 450 nm subtracted from the net antibody binding signal.

FIGS. 32A-32B depict quantification of Membrane Proteome Array (MPA) screening of AB2 binding to proteins in an MPA protein library (FIG. 32A) and mean immunofluorescence intensity of AB2 to targets identified by MPA screening by flow cytometry using serial dilutions of AB2 (FIG. 32B).

FIGS. 33A-33B depict quantification of Membrane Proteome Array (MPA) screening of AB1 binding to proteins in an MPA protein library (FIG. 33A) and mean immunofluorescence intensity of AB1 to targets identified by MPA screening by flow cytometry using serial dilutions of AB1 (FIG. 33B).

FIG. 34A and FIG. 34B show serum concentration curves of AB4 and AB2 antibodies, respectively, after a single intravenous dose of either 0.3 mg/kg, 1 mg/kg, or 3 mg/kg in an amyloidoma mouse model, as measured by modified sandwich-ELISA on a Meso Scale Discovery (MSD) platform. Each plotted point represents the mean SD of 5 animals.

### DETAILED DESCRIPTION

Current treatments for amyloid light chain (AL) amyloidosis are therapies designed to treat the underlying plasma cell dyscrasia (i.e., multiple myeloma) by targeting the cells that produce the toxic light chains. The only on-label therapy currently available is the monoclonal antibody-based therapy daratumumab and hyaluronidase-fihj in combination with bortezomib, cyclophosphamide and dexamethasone. This antibody (and other off-label therapies) targets the CD38 receptor on plasma cells to induce antibody-dependent cellular cytotoxicity. The drug's initial indication is multiple myeloma, but it was approved as an on-label treatment for newly diagnosed AL amyloidosis patients in January 2021. Other treatments for AL amyloidosis include more invasive interventions such as autologous stem cell transplants (ASTC) or standard cytotoxic chemotherapies. However, all the preceding therapies work by targeting the proliferation of the disease-causing plasma cell clone and do not address the already-present amyloid deposits directly affecting organs.

While effective for early-stage patients, daratumumab is also not recommended for patients with late-stage cardiac disease. Thus, a non-invasive therapy that does not deplete plasma cell populations to address the existing toxic amyloid deposits to improve organ function of late-stage patients remains a significant challenge and unmet need in treating AL amyloidosis and/or disorders characterized by misfolded antibody molecule.

### Anti-human κ Light Chain Antibodies

Provided herein are antibodies and antibody fragments that bind human kappa (x) light chain. The anti-human κ light chain antibodies described herein are useful and advantageous in that the antibodies and antibody fragments specifically bind to misfolded κ light chains (i.e., as compared to folded κ light chains). Generally, their useful and advantageous can be attributed to the ability of the anti-human κ light chain antibodies to specifically bind to an epitope specific to misfolded κ light chains, SEQ ID NO: 33. The antibodies described herein are anti-human κ light chain antibodies that, in certain instances, promote the clearance of misfolded κ light chains (e.g., present in AL amyloidosis or a plasma cell disorder). These anti-human κ light chain antibodies, in some cases, increase uptake of misfolded κ light chain aggregates and/or recruit phagocytes. In some embodiments, the anti-human κ light chain antibodies are useful in methods for reducing amyloid deposits and/or treating diseases characterized by amyloid deposits (e.g., present in AL amyloidosis or a plasma cell disorder). In other instances, the anti-human κ light chain antibodies can be used to diagnose, detect, or screen for misfolded κ light chains, amyloid deposits, and/or a disorder associated therewith.

### Anti-Human κ Light Chain CDRs

Provided and described herein are anti-human κ light chain antibodies and/or human κ light chain binding antibody fragments comprising:
(a) a heavy chain complementarity determining region 1 (H-CDR1) comprising an amino acid sequence set forth in SEQ ID NO: 2;
(b) a heavy chain complementarity determining region 2 (H-CDR2) comprising an amino acid sequence set forth in SEQ ID NO: 3;
(c) a heavy chain complementarity determining region 3 (H-CDR3) comprising an amino acid sequence set forth in SEQ ID NO: 4;
(d) a light chain complementarity determining region 1 (L-CDR1) comprising an amino acid sequence set forth in SEQ ID NO: 6;
(e) a light chain complementarity determining region 2 (L-CDR2) comprising an amino acid sequence set forth in SEQ ID NO: 7; and
(f) a light chain complementarity determining region 3 (L-CDR3) comprising an amino acid sequence set forth in SEQ ID NO: 8.

In some embodiments, the anti-human κ light chain antibodies and/or anti-human κ light chain binding antibody fragments comprise: a heavy chain variable region and/or a light chain variable region. In some embodiments, the heavy chain variable region comprises an amino acid sequence having at least about 70%, at least about 80%, at least about 85%, at least about 90%, at least about 95%, at least about 96%, at least about 97%, at least about 98%, or at least about 99% identity to SEQ ID NO: 1, and comprises an H-CDR1, H-CDR2, and H-CDR3 as described in amino acid sequences set forth in SEQ ID Nos: 2, 3, and 4, respectively; and the light chain variable region comprises an amino acid sequence having at least about 70%, at least about 80%, at least about 85%, at least about 90%, at least about 95%, at least about 96%, at least about 97%, at least about 98%, or at least about 99% identity to SEQ ID NO: 5, and comprises an L-CDR1, L-CDR2, and L-CDR3 as described in amino acid sequences set forth in SEQ ID Nos: 6, 7, and 8, respectively. In some embodiments, the heavy chain variable region comprises an amino acid sequence corresponding to SEQ ID NO: 1, and the light chain variable region comprises an amino acid sequence corresponding to SEQ ID NO: 5. In some embodiments, the anti-human κ light chain antibodies and/or human κ light chain binding antibody fragments binds an epitope comprising SEQ ID NO: 33. In some embodiments, the heavy chain variable region comprises the amino acid sequence of SEQ ID NO: 1; or the light chain variable region comprises the amino acid sequence of SEQ ID NO: 5. In some cases, the heavy chain variable region comprises (i) an amino acid sequence having at least about 80% sequence identity to an amino acid sequence of SEQ ID NO: 1, and (ii) an H-CDR1, H-CDR2, and H-CDR3 as described in the amino acid sequences set forth in SEQ ID Nos: 2, 3, and 4, respectively; and the light chain variable region comprises (i) an amino acid sequence having at least about 80% sequence identity to an amino acid sequence of SEQ ID NO: 5, and (ii) an L-CDR1, L-CDR2, and L-CDR3 as described in the amino acid sequences set forth in SEQ ID Nos: 6, 7, and 8, respectively. In some cases, the heavy chain variable region comprises (i) an amino acid sequence having at least about 85% sequence identity to an amino acid sequence of SEQ ID NO: 1, and (ii) an H-CDR1, H-CDR2, and H-CDR3 as described in the amino acid sequences set forth in SEQ ID Nos: 2, 3, and 4, respectively; and the light chain variable region comprises (i) an amino acid sequence having at least about 85% sequence identity to an amino acid sequence of SEQ ID NO: 5, and (ii) an L-CDR1, L-CDR2, and L-CDR3 as described in the amino acid sequences set forth in SEQ ID Nos: 6, 7, and 8, respectively. In some cases, the heavy chain variable region comprises (i) an amino acid sequence having at least about 90% sequence identity to an amino acid sequence of SEQ ID NO: 1, and (ii) an H-CDR1, H-CDR2, and H-CDR3 as described in the amino acid sequences set forth in SEQ ID Nos: 2, 3, and 4, respectively; and the light chain variable region comprises (i) an amino acid sequence having at least about 90% sequence identity to an amino acid sequence of SEQ ID NO: 5, and (ii) an L-CDR1, L-CDR2, and L-CDR3 as described in the amino acid sequences set forth in SEQ ID Nos: 6, 7, and 8, respectively. In some cases, the heavy chain variable region comprises (i) an amino acid sequence having at least about 95% sequence identity to an amino acid sequence of SEQ ID NO: 1, and (ii) an H-CDR1, H-CDR2, and H-CDR3 as described in the amino acid sequences set forth in SEQ ID Nos: 2, 3, and 4, respectively; and the light chain variable region comprises (i) an amino acid sequence having at least about 95% sequence identity to an amino acid sequence of SEQ ID NO: 5, and (ii) an L-CDR1, L-CDR2, and L-CDR3 as described in the amino acid sequences set forth in SEQ ID Nos: 6, 7, and 8, respectively. In some cases, the heavy chain variable region comprises (i) an amino acid sequence having at least about 97% sequence identity to an amino acid sequence of SEQ ID NO: 1, and (ii) an H-CDR1, H-CDR2, and H-CDR3 as described in the amino acid sequences set forth in SEQ ID Nos: 2, 3, and 4, respectively; and the light chain variable region comprises (i) an amino acid sequence having at least about 97% sequence identity to an amino acid sequence of SEQ ID NO: 5, and (ii) an L-CDR1, L-CDR2, and L-CDR3 as described in the amino acid sequences set forth in SEQ ID Nos: 6, 7, and 8, respectively. In some cases, the heavy chain variable region comprises (i) an amino acid sequence having at least about 99% sequence identity to an amino acid sequence of SEQ ID NO: 1, and (ii) an H-CDR1, H-CDR2, and H-CDR3 as described in the amino acid sequences set forth in SEQ ID Nos: 2, 3, and 4, respectively; and the light chain variable region comprises (i) an amino acid sequence having at least about 99% sequence identity to an amino acid sequence of SEQ ID NO: 5, and (ii) an L-CDR1, L-CDR2, and L-CDR3 as described in the amino acid sequences set forth in SEQ ID Nos: 6, 7, and 8, respectively.

Also provided herein is an anti-human kappa light chain antibody and human kappa light chain binding fragment thereof comprising: (a) a heavy chain variable region comprising an amino acid sequence of SEQ ID NO: 1; and (b) a light chain variable region comprising an amino acid sequence of SEQ ID NO: 5.

Also provided and described herein are anti-human κ light chain antibodies and/or human κ light chain binding antibody fragments comprising:
(a) a heavy chain complementarity determining region 1 (H-CDR1) comprising an amino acid sequence set forth in SEQ ID NO: 10;
(b) a heavy chain complementarity determining region 2 (H-CDR2) comprising an amino acid sequence set forth in SEQ ID NO: 11;
(c) a heavy chain complementarity determining region 3 (H-CDR3) comprising an amino acid sequence set forth in SEQ ID NO: 12;
(d) a light chain complementarity determining region 1 (L-CDR1) comprising an amino acid sequence set forth in SEQ ID NO: 14;
(e) a light chain complementarity determining region 2 (L-CDR2) comprising an amino acid sequence set forth in SEQ ID NO: 15; and
(f) a light chain complementarity determining region 3 (L-CDR3) comprising an amino acid sequence set forth in SEQ ID NO: 16.

In some embodiments, the anti-human κ light chain antibodies and/or anti-human κ light chain binding antibody fragments comprise: a heavy chain variable region and a and/or a light chain variable region. In some embodiments, the heavy chain variable region comprises an amino acid sequence having at least about 70%, at least about 80%, at least about 85%, at least about 90%, at least about 95%, or at least about 98% identity to SEQ ID NO: 9, and comprises an H-CDR1, H-CDR2, and H-CDR3 as described in amino acid sequences set forth in SEQ ID Nos: 10, 11, and 12, respectively; and the light chain variable region comprises an amino acid sequence having at least about 70%, at least about 80%, at least about 85%, at least about 90%, at least about 95%, or at least about 98% identity to SEQ ID NO: 13, and comprises an L-CDR1, L-CDR2, and L-CDR3 as described in amino acid sequences set forth in SEQ ID Nos: 14, 15, and 16, respectively. In some embodiments, the heavy chain variable region comprises an amino acid sequence corresponding to SEQ ID NO: 9, and the light chain variable region comprises an amino acid sequence corresponding to SEQ ID NO: 13. In some embodiments, the anti-human κ light chain antibodies and/or human κ light chain binding antibody fragments bind an epitope comprising SEQ ID NO: 33. In some embodiments, the heavy chain variable region comprises the amino acid sequence of SEQ ID NO: 9; or the light chain variable region comprises the amino acid sequence of SEQ ID NO: 13. In some cases, the heavy chain variable region comprises (i) an amino acid sequence having at least about 80% sequence identity to an amino acid sequence of SEQ ID NO: 9, and (ii) an H-CDR1, H-CDR2, and H-CDR3 as described in the amino acid sequences set forth in SEQ ID Nos: 10, 11, and 12, respectively; and the light chain variable region comprises (i) an amino acid sequence having at least about 80% sequence identity to an amino acid sequence of SEQ ID NO: 13, and (ii) an L-CDR1, L-CDR2, and L-CDR3 as described in the amino acid sequences set forth in SEQ ID Nos: 14, 15, and 16, respectively. In some cases, the heavy chain variable region comprises (i) an amino acid sequence having at least about 85% sequence identity to an amino acid sequence of SEQ ID NO: 9, and (ii) an H-CDR1, H-CDR2, and H-CDR3 as described in the amino acid sequences set forth in SEQ ID Nos: 10, 11, and 12, respectively; and the light chain variable region comprises (i) an amino acid sequence having at least about 85% sequence identity to an amino acid sequence of SEQ ID NO: 13, and (ii) an L-CDR1, L-CDR2, and L-CDR3 as described in the amino acid sequences set forth in SEQ ID Nos: 14, 15, and 16, respectively. In some cases, the heavy chain variable region comprises (i) an amino acid sequence having at least about 90% sequence identity to an amino acid sequence of SEQ ID NO: 9, and (ii) an H-CDR1, H-CDR2, and H-CDR3 as described in the amino acid sequences set forth in SEQ ID Nos: 10, 11, and 12, respectively; and the light chain variable region comprises (i) an amino acid sequence having at least about 90% sequence identity to an amino acid sequence of SEQ ID NO: 13, and (ii) an L-CDR1, L-CDR2, and L-CDR3 as described in the amino acid sequences set forth in SEQ ID Nos: 14, 15, and 16, respectively. In some cases, the heavy chain variable region comprises (i) an amino acid sequence having at least about 95% sequence identity to an amino acid sequence of SEQ ID NO: 9, and (ii) an H-CDR1, H-CDR2, and H-CDR3 as described in the amino acid sequences set forth in SEQ ID Nos: 10, 11, and 12, respectively; and the light chain variable region comprises (i) an amino acid sequence having at least about 95% sequence identity to an amino acid sequence of SEQ ID NO: 13, and (ii) an L-CDR1, L-CDR2, and L-CDR3 as described in the amino acid sequences set forth in SEQ ID Nos: 14, 15, and 16, respectively. In some cases, the heavy chain variable region comprises (i) an amino acid sequence having at least about 97% sequence identity to an amino acid sequence of SEQ ID NO: 9, and (ii) an H-CDR1, H-CDR2, and H-CDR3 as described in the amino acid sequences set forth in SEQ ID Nos: 10, 11, and 12, respectively; and the light chain variable region comprises (i) an amino acid sequence having at least about 97% sequence identity to an amino acid sequence of SEQ ID NO: 13, and (ii) an L-CDR1, L-CDR2, and L-CDR3 as described in the amino acid sequences set forth in SEQ ID Nos: 14, 15, and 16, respectively. In some cases, the heavy chain variable region comprises (i) an amino acid sequence having at least about 99% sequence identity to an amino acid sequence of SEQ ID NO: 9, and (ii) an H-CDR1, H-CDR2, and H-CDR3 as described in the amino acid sequences set forth in SEQ ID Nos: 10, 11, and 12, respectively; and the light chain variable region comprises (i) an amino acid sequence having at least about 99% sequence identity to an amino acid sequence of SEQ ID NO: 13, and (ii) an L-CDR1, L-CDR2, and L-CDR3 as described in the amino acid sequences set forth in SEQ ID Nos: 14, 15, and 16, respectively.

Also provided herein is an anti-human kappa light chain antibody and human kappa light chain binding fragment thereof comprising: (a) a heavy chain variable region comprising an amino acid sequence of SEQ ID NO: 9; and (b) a light chain variable region comprising an amino acid sequence of SEQ ID NO: 13.

Further provided and described herein are anti-human κ light chain antibodies and/or human κ light chain binding antibody fragments comprising:
(a) a heavy chain complementarity determining region 1 (H-CDR1) comprising an amino acid sequence set forth in SEQ ID NO: 18;
(b) a heavy chain complementarity determining region 2 (H-CDR2) comprising an amino acid sequence set forth in SEQ ID NO: 19;
(c) a heavy chain complementarity determining region 3 (H-CDR3) comprising an amino acid sequence set forth in SEQ ID NO: 20;
(d) a light chain complementarity determining region 1 (L-CDR1) comprising an amino acid sequence set forth in SEQ ID NO: 22;
(e) a light chain complementarity determining region 2 (L-CDR2) comprising an amino acid sequence set forth in SEQ ID NO: 23; and
(f) a light chain complementarity determining region 3 (L-CDR3) comprising an amino acid sequence set forth in SEQ ID NO: 24.

In some embodiments, the anti-human κ light chain antibodies and/or human κ light chain binding antibody fragments comprise: a heavy chain variable region and a and/or a light chain variable region. In some embodiments, the heavy chain variable region comprises an amino acid sequence having at least about 70%, at least about 80%, at least about 85%, at least about 90%, at least about 95%, at least about 96%, at least about 97%, at least about 98%, or at least about 99% identity to SEQ ID NO: 17, and comprises an H-CDR1, H-CDR2, and H-CDR3 as described in amino acid sequences set forth in SEQ ID Nos: 18, 19, and 20, respectively; and/or the light chain variable region comprises an amino acid sequence having at least about 70%, at least about 80%, at least about 85%, at least about 90%, at least about 95%, at least about 96%, at least about 97%, at least about 98%, or at least about 99% identity to SEQ ID NO: 21, and comprises an L-CDR1, L-CDR2, and L-CDR3 as described in amino acid sequences set forth in SEQ ID Nos: 22, 23, and 24, respectively. In some embodiments, the heavy chain variable region comprises an amino acid sequence corresponding to SEQ ID NO: 17, and the light chain variable region comprises an amino acid sequence corresponding to SEQ ID NO: 21. In some embodiments, the anti-human κ light chain antibodies and/or human κ light chain binding antibody fragments bind an epitope comprising SEQ ID NO: 33. In some embodiments, the heavy chain variable region comprises the amino acid sequence of SEQ ID NO: 17; or the light chain variable region comprises the amino acid sequence of SEQ ID NO: 21. In some cases, the heavy chain variable region comprises (i) an amino acid sequence having at least about 80% sequence identity to an amino acid sequence of SEQ ID NO: 17, and (ii) an H-CDR1, H-CDR2, and H-CDR3 as described in the amino acid sequences set forth in SEQ ID Nos: 18, 19, and 20, respectively; and the light chain variable region comprises (i) an amino acid sequence having at least about 80% sequence identity to an amino acid sequence of SEQ ID NO: 21, and (ii) an L-CDR1, L-CDR2, and L-CDR3 as described in the amino acid sequences set forth in SEQ ID Nos: 22, 23, and 24, respectively. In some cases, the heavy chain variable region comprises (i) an amino acid sequence having at least about 85% sequence identity to an amino acid sequence of SEQ ID NO: 17, and (ii) an H-CDR1, H-CDR2, and H-CDR3 as described in the amino acid sequences set forth in SEQ ID Nos: 18, 19, and 20, respectively; and the light chain variable region comprises (i) an amino acid sequence having at least about 85% sequence identity to an amino acid sequence of SEQ ID NO: 21, and (ii) an L-CDR1, L-CDR2, and L-CDR3 as described in the amino acid sequences set forth in SEQ ID Nos: 22, 23, and 24, respectively. In some cases, the heavy chain variable region comprises (i) an amino acid sequence having at least about 90% sequence identity to an amino acid sequence of SEQ ID NO: 17, and (ii) an H-CDR1, H-CDR2, and H-CDR3 as described in the amino acid sequences set forth in SEQ ID Nos: 18, 19, and 20, respectively; and the light chain variable region comprises (i) an amino acid sequence having at least about 90% sequence identity to an amino acid sequence of SEQ ID NO: 21, and (ii) an L-CDR1, L-CDR2, and L-CDR3 as described in the amino acid sequences set forth in SEQ ID Nos: 22, 23, and 24, respectively. In some cases, the heavy chain variable region comprises (i) an amino acid sequence having at least about 95% sequence identity to an amino acid sequence of SEQ ID NO: 17, and (ii) an H-CDR1, H-CDR2, and H-CDR3 as described in the amino acid sequences set forth in SEQ ID Nos: 18, 19, and 20, respectively; and the light chain variable region comprises (i) an amino acid sequence having at least about 95% sequence identity to an amino acid sequence of SEQ ID NO: 21, and (ii) an L-CDR1, L-CDR2, and L-CDR3 as described in the amino acid sequences set forth in SEQ ID Nos: 22, 23, and 24, respectively. In some cases, the heavy chain variable region comprises (i) an amino acid sequence having at least about 97% sequence identity to an amino acid sequence of SEQ ID NO: 17, and (ii) an H-CDR1, H-CDR2, and H-CDR3 as described in the amino acid sequences set forth in SEQ ID Nos: 18, 19, and 20, respectively; and the light chain variable region comprises (i) an amino acid sequence having at least about 97% sequence identity to an amino acid sequence of SEQ ID NO: 21, and (ii) an L-CDR1, L-CDR2, and L-CDR3 as described in the amino acid sequences set forth in SEQ ID Nos: 22, 23, and 24, respectively. In some cases, the heavy chain variable region comprises (i) an amino acid sequence having at least about 99% sequence identity to an amino acid sequence of SEQ ID NO: 17, and (ii) an H-CDR1, H-CDR2, and H-CDR3 as described in the amino acid sequences set forth in SEQ ID Nos: 18, 19, and 20, respectively; and the light chain variable region comprises (i) an amino acid sequence having at least about 99% sequence identity to an amino acid sequence of SEQ ID NO: 21, and (ii) an L-CDR1, L-CDR2, and L-CDR3 as described in the amino acid sequences set forth in SEQ ID Nos: 22, 23, and 24, respectively.

Also provided herein is an anti-human kappa light chain antibody and human kappa light chain binding fragment thereof comprising: (a) a heavy chain variable region comprising an amino acid sequence of SEQ ID NO: 17; and (b) a light chain variable region comprising an amino acid sequence of SEQ ID NO: 21.

Provided and described herein are anti-human κ light chain antibodies and/or human κ light chain binding antibody fragments comprising:
(a) a heavy chain complementarity determining region 1 (H-CDR1) comprising an amino acid sequence set forth in SEQ ID NO: 26;
(b) a heavy chain complementarity determining region 2 (H-CDR2) comprising an amino acid sequence set forth in SEQ ID NO: 27;
(c) a heavy chain complementarity determining region 3 (H-CDR3) comprising an amino acid sequence set forth in SEQ ID NO: 28;
(d) a light chain complementarity determining region 1 (L-CDR1) comprising an amino acid sequence set forth in SEQ ID NO: 30;
(e) a light chain complementarity determining region 2 (L-CDR2) comprising an amino acid sequence set forth in SEQ ID NO: 31; and
(f) a light chain complementarity determining region 3 (L-CDR3) comprising an amino acid sequence set forth in SEQ ID NO: 32.

In some embodiments, the anti-human κ light chain antibodies and/or human κ light chain binding antibody fragments comprise: a heavy chain variable region and a and/or a light chain variable region. In some embodiments, the heavy chain variable region comprises an amino acid sequence having at least about 70%, at least about 80%, at least about 85%, at least about 90%, at least about 95%, at least about 96%, at least about 97%, at least about 98%, or at least about 99% identity to SEQ ID NO: 25, and comprises an H-CDR1, H-CDR2, and H-CDR3 as described in amino acid sequences set forth in SEQ ID Nos: 26, 27, and 28, respectively; and/or the light chain variable region comprises an amino acid sequence having at least about 70%, at least about 80%, at least about 85%, at least about 90%, at least about 95%, at least about 96%, at least about 97%, at least about 98%, or at least about 99% identity to SEQ ID NO: 29, and comprises an L-CDR1, L-CDR2, and L-CDR3 as described in amino acid sequences set forth in SEQ ID Nos: 30, 31, and 32, respectively. In some embodiments, the heavy chain variable region comprises an amino acid sequence corresponding to SEQ ID NO: 25, and the light chain variable region comprises an amino acid sequence corresponding to SEQ ID NO: 29. In some embodiments, the anti-human κ light chain antibodies and/or human κ light chain binding antibody fragments bind an epitope comprising SEQ ID NO: 33. In some embodiments, the heavy chain variable region comprises the amino acid sequence of SEQ ID NO: 25; or the light chain variable region comprises the amino acid sequence of SEQ ID NO: 29. In some cases, the heavy chain variable region comprises (i) an amino acid sequence having at least about 80% sequence identity to an amino acid sequence of SEQ ID NO: 25, and (ii) an H-CDR1, H-CDR2, and H-CDR3 as described in the amino acid sequences set forth in SEQ ID Nos: 26, 27, and 28, respectively; and the light chain variable region comprises (i) an amino acid sequence having at least about 80% sequence identity to an amino acid sequence of SEQ ID NO: 29, and (ii) an L-CDR1, L-CDR2, and L-CDR3 as described in the amino acid sequences set forth in SEQ ID Nos: 30, 31, and 32, respectively. In some cases, the heavy chain variable region comprises (i) an amino acid sequence having at least about 85% sequence identity to an amino acid sequence of SEQ ID NO: 25, and (ii) an H-CDR1, H-CDR2, and H-CDR3 as described in the amino acid sequences set forth in SEQ ID Nos: 26, 27, and 28, respectively; and the light chain variable region comprises (i) an amino acid sequence having at least about 85% sequence identity to an amino acid sequence of SEQ ID NO: 29, and (ii) an L-CDR1, L-CDR2, and L-CDR3 as described in the amino acid sequences set forth in SEQ ID Nos: 30, 31, and 32, respectively. In some cases, the heavy chain variable region comprises (i) an amino acid sequence having at least about 90% sequence identity to an amino acid sequence of SEQ ID NO: 25, and (ii) an H-CDR1, H-CDR2, and H-CDR3 as described in the amino acid sequences set forth in SEQ ID Nos: 26, 27, and 28, respectively; and the light chain variable region comprises (i) an amino acid sequence having at least about 90% sequence identity to an amino acid sequence of SEQ ID NO: 29, and (ii) an L-CDR1, L-CDR2, and L-CDR3 as described in the amino acid sequences set forth in SEQ ID Nos: 30, 31, and 32, respectively. In some cases, the heavy chain variable region comprises (i) an amino acid sequence having at least about 95% sequence identity to an amino acid sequence of SEQ ID NO: 25, and (ii) an H-CDR1, H-CDR2, and H-CDR3 as described in the amino acid sequences set forth in SEQ ID Nos: 26, 27, and 28, respectively; and the light chain variable region comprises (i) an amino acid sequence having at least about 95% sequence identity to an amino acid sequence of SEQ ID NO: 29, and (ii) an L-CDR1, L-CDR2, and L-CDR3 as described in the amino acid sequences set forth in SEQ ID Nos: 30, 31, and 32, respectively. In some cases, the heavy chain variable region comprises (i) an amino acid sequence having at least about 97% sequence identity to an amino acid sequence of SEQ ID NO: 25, and (ii) an H-CDR1, H-CDR2, and H-CDR3 as described in the amino acid sequences set forth in SEQ ID Nos: 26, 27, and 28, respectively; and the light chain variable region comprises (i) an amino acid sequence having at least about 97% sequence identity to an amino acid sequence of SEQ ID NO: 29, and (ii) an L-CDR1, L-CDR2, and L-CDR3 as described in the amino acid sequences set forth in SEQ ID Nos: 30, 31, and 32, respectively. In some cases, the heavy chain variable region comprises (i) an amino acid sequence having at least about 99% sequence identity to an amino acid sequence of SEQ ID NO: 25, and (ii) an H-CDR1, H-CDR2, and H-CDR3 as described in the amino acid sequences set forth in SEQ ID Nos: 26, 27, and 28, respectively; and the light chain variable region comprises (i) an amino acid sequence having at least about 99% sequence identity to an amino acid sequence of SEQ ID NO: 29, and (ii) an L-CDR1, L-CDR2, and L-CDR3 as described in the amino acid sequences set forth in SEQ ID Nos: 30, 31, and 32, respectively.

Also provided herein is an anti-human kappa light chain antibody and human kappa light chain binding fragment thereof comprising: (a) a heavy chain variable region comprising an amino acid sequence of SEQ ID NO: 25; and (b) a light chain variable region comprising an amino acid sequence of SEQ ID NO: 29.

Provided and described herein are anti-human κ light chain antibodies and/or human κ light chain binding antibody fragments comprising:
(a) a heavy chain complementarity determining region 1 (H-CDR1);
(b) a heavy chain complementarity determining region 2 (H-CDR2);
(c) a heavy chain complementarity determining region 3 (H-CDR3);
(d) a light chain complementarity determining region 1 (L-CDR1);
(e) a light chain complementarity determining region 2 (L-CDR2); and/or
(f) a light chain complementarity determining region 3 (L-CDR3).

In some embodiments, the H-CDR1, the H-CDR2, and the H-CDR3 are derived from SEQ ID NO: 1, and the L-CDR1, the L-CDR2, and the L-CDR3 derived from SEQ ID NO: 5; and wherein the H-CDR1, the H-CDR2, the H-CDR3, L-CDR1, the L-CDR2, and the L-CDR3 are defined using Chothia, Kabat, IMGT, Contact, or AbM methodologies. In some embodiments, the H-CDR1, the H-CDR2, the H-CDR3, L-CDR1, the L-CDR2, and the L-CDR3 are defined using Chothia. In some embodiments, the H-CDR1, the H-CDR2, the H-CDR3, L-CDR1, the L-CDR2, and the L-CDR3 are defined using Kabat. In some embodiments, the H-CDR1, the H-CDR2, the H-CDR3, L-CDR1, the L-CDR2, and the L-CDR3 are defined using IMGT. In some embodiments, the H-CDR1, the H-CDR2, the H-CDR3, L-CDR1, the L-CDR2, and the L-CDR3 are defined using Contact. In some embodiments, the H-CDR1, the H-CDR2, the H-CDR3, L-CDR1, the L-CDR2, and the L-CDR3 are defined using AbM.

In some embodiments, the H-CDR1, the H-CDR2, and the H-CDR3 are derived from SEQ ID NO: 9, and the L-CDR1, the L-CDR2, and the L-CDR3 derived from SEQ ID NO: 13; and wherein the H-CDR1, the H-CDR2, the H-CDR3, L-CDR1, the L-CDR2, and the L-CDR3 are defined using Chothia, Kabat, IMGT, Contact, or AbM methodologies. In some embodiments, the H-CDR1, the H-CDR2, the H-CDR3, L-CDR1, the L-CDR2, and the L-CDR3 are defined using Chothia. In some embodiments, the H-CDR1, the H-CDR2, the H-CDR3, L-CDR1, the L-CDR2, and the L-CDR3 are defined using Kabat. In some embodiments, the H-CDR1, the H-CDR2, the H-CDR3, L-CDR1, the L-CDR2, and the L-CDR3 are defined using IMGT. In some embodiments, the H-CDR1, the H-CDR2, the H-CDR3, L-CDR1, the L-CDR2, and the L-CDR3 are defined using Contact. In some embodiments, the H-CDR1, the H-CDR2, the H-CDR3, L-CDR1, the L-CDR2, and the L-CDR3 are defined using AbM.

In some embodiments, the H-CDR1, the H-CDR2, and the H-CDR3 are derived from SEQ ID NO: 17, and the L-CDR1, the L-CDR2, and the L-CDR3 derived from SEQ ID NO: 21; and wherein the H-CDR1, the H-CDR2, the H-CDR3, L-CDR1, the L-CDR2, and the L-CDR3 are defined using Chothia, Kabat, IMGT, Contact, or AbM methodologies. In some embodiments, the H-CDR1, the H-CDR2, the H-CDR3, L-CDR1, the L-CDR2, and the L-CDR3 are defined using Chothia. In some embodiments, the H-CDR1, the H-CDR2, the H-CDR3, L-CDR1, the L-CDR2, and the L-CDR3 are defined using Kabat. In some embodiments, the H-CDR1, the H-CDR2, the H-CDR3, L-CDR1, the L-CDR2, and the L-CDR3 are defined using IMGT. In some embodiments, the H-CDR1, the H-CDR2, the H-CDR3, L-CDR1, the L-CDR2, and the L-CDR3 are defined using Contact. In some embodiments, the H-CDR1, the H-CDR2, the H-CDR3, L-CDR1, the L-CDR2, and the L-CDR3 are defined using AbM.

In some embodiments, the H-CDR1, the H-CDR2, and the H-CDR3 are derived from SEQ ID NO: 25, and the L-CDR1, the L-CDR2, and the L-CDR3 derived from SEQ ID NO: 29; and wherein the H-CDR1, the H-CDR2, the H-CDR3, L-CDR1, the L-CDR2, and the L-CDR3 are defined using Chothia, Kabat, IMGT, Contact, or AbM methodologies. In some embodiments, the H-CDR1, the H-CDR2, the H-CDR3, L-CDR1, the L-CDR2, and the L-CDR3 are defined using Chothia. In some embodiments, the H-CDR1, the H-CDR2, the H-CDR3, L-CDR1, the L-CDR2, and the L-CDR3 are defined using Kabat. In some embodiments, the H-CDR1, the H-CDR2, the H-CDR3, L-CDR1, the L-CDR2, and the L-CDR3 are defined using IMGT. In some embodiments, the H-CDR1, the H-CDR2, the H-CDR3, L-CDR1, the L-CDR2, and the L-CDR3 are defined using Contact. In some embodiments, the H-CDR1, the H-CDR2, the H-CDR3, L-CDR1, the L-CDR2, and the L-CDR3 are defined using AbM.

In some embodiments, the anti-human K light chain antibodies and/or human K light chain binding antibody fragments bind to an epitope comprising SEQ ID NO: 33.

In certain embodiments, the anti-human K light chain antibodies and/or human K light chain binding antibody fragments bind to an epitope consisting of SEQ ID NO: 33. In certain embodiments, binding is measured by ELISA, surface plasmon resonance, bio-layer interferometry, or isothermal calorimetry.

In some embodiments, the anti-human K light chain antibodies and/or human K light chain binding antibody fragments further comprise a heavy chain framework region 1 (H-FR1), a heavy chain framework region 2 (H-FR2), a heavy chain framework region 3 (H-FR3), and a heavy chain framework region 4 (H-FR4); and a light chain framework region 1 (L-FR1), a light chain framework region 2 (L-FR2), a light chain framework region 3 (L-FR3), and a light chain framework region 4 (L-FR4). In some embodiments, the anti-human K light chain antibodies and/or human κ light chain binding antibody fragments further comprise a heavy chain variable region and a light chain variable region. In some embodiments, the anti-human κ light chain antibodies and/or human κ light chain binding antibody fragments further comprise a heavy chain constant region and a light chain constant region.

In some embodiments, the anti-human κ light chain antibodies and/or human κ light chain binding antibody fragments comprise a Fab, a Fab′, a F(ab′)₂, a scFv, a dsFv, a ds-scFv, a dimer, a minibody, or a diabody.

### Anti-Human κ Light Chain Variable Regions

Provided and described herein are anti-human κ light chain antibodies and/or human κ light chain binding antibody fragments comprising:
a heavy chain variable region comprising an amino acid sequence having at least about 70%, at least about 80%, at least about 85%, at least about 90%, at least about 95%, at least about 96%, at least about 97%, at least about 98%, or at least about 99% identity to SEQ ID NO: 1, and comprising an H-CDR1, H-CDR2, and H-CDR3 as described in amino acid sequences set forth in SEQ ID Nos: 2, 3, and 4, respectively; and/or
a light chain variable region comprising an amino acid sequence having at least about 70%, at least about 80%, at least about 85%, at least about 90%, at least about 95%, at least about 96%, at least about 97%, at least about 98%, or at least about 99% identity to SEQ ID NO: 5, and comprising a L-CDR1, L-CDR2, and L-CDR3 as described in amino acid sequences set forth in SEQ ID Nos: 6, 7, and 8, respectively;
wherein the anti-human κ light chain antibodies and/or human κ light chain binding antibody fragments bind an epitope comprising SEQ ID NO: 33 (e.g., as measured by an ELISA).

In some cases, the heavy chain variable region comprises (i) an amino acid sequence having at least about 80% sequence identity to an amino acid sequence of SEQ ID NO: 1, and (ii) an H-CDR1, H-CDR2, and H-CDR3 as described in the amino acid sequences set forth in SEQ ID Nos: 2, 3, and 4, respectively. In some cases, the heavy chain variable region comprises (i) an amino acid sequence having at least about 85% sequence identity to an amino acid sequence of SEQ ID NO: 1, and (ii) an H-CDR1, H-CDR2, and H-CDR3 as described in the amino acid sequences set forth in SEQ ID Nos: 2, 3, and 4, respectively. In some cases, the heavy chain variable region comprises (i) an amino acid sequence having at least about 90% sequence identity to an amino acid sequence of SEQ ID NO: 1, and (ii) an H-CDR1, H-CDR2, and H-CDR3 as described in the amino acid sequences set forth in SEQ ID Nos: 2, 3, and 4, respectively. In some cases, the heavy chain variable region comprises (i) an amino acid sequence having at least about 95% sequence identity to an amino acid sequence of SEQ ID NO: 1, and (ii) an H-CDR1, H-CDR2, and H-CDR3 as described in the amino acid sequences set forth in SEQ ID Nos: 2, 3, and 4, respectively. In some cases, the heavy chain variable region comprises (i) an amino acid sequence having at least about 97% sequence identity to an amino acid sequence of SEQ ID NO: 1, and (ii) an H-CDR1, H-CDR2, and H-CDR3 as described in the amino acid sequences set forth in SEQ ID Nos: 2, 3, and 4, respectively. In some cases, the heavy chain variable region comprises (i) an amino acid sequence having at least about 99% sequence identity to an amino acid sequence of SEQ ID NO: 1, and (ii) an H-CDR1, H-CDR2, and H-CDR3 as described in the amino acid sequences set forth in SEQ ID Nos: 2, 3, and 4, respectively.

In some cases, the light chain variable region comprises (i) an amino acid sequence having at least about 80% sequence identity to an amino acid sequence of SEQ ID NO: 5, and (ii) an L-CDR1, L-CDR2, and L-CDR3 as described in the amino acid sequences set forth in SEQ ID Nos: 6, 7, and 8, respectively. In some cases, the light chain variable region comprises (i) an amino acid sequence having at least about 85% sequence identity to an amino acid sequence of SEQ ID NO: 5, and (ii) an L-CDR1, L-CDR2, and L-CDR3 as described in the amino acid sequences set forth in SEQ ID Nos: 6, 7, and 8, respectively. In some cases, the light chain variable region comprises (i) an amino acid sequence having at least about 90% sequence identity to an amino acid sequence of SEQ ID NO: 5, and (ii) an L-CDR1, L-CDR2, and L-CDR3 as described in the amino acid sequences set forth in SEQ ID Nos: 6, 7, and 8, respectively. In some cases, the light chain variable region comprises (i) an amino acid sequence having at least about 95% sequence identity to an amino acid sequence of SEQ ID NO: 5, and (ii) an L-CDR1, L-CDR2, and L-CDR3 as described in the amino acid sequences set forth in SEQ ID Nos: 6, 7, and 8, respectively. In some cases, the light chain variable region comprises (i) an amino acid sequence having at least about 97% sequence identity to an amino acid sequence of SEQ ID NO: 5, and (ii) an L-CDR1, L-CDR2, and L-CDR3 as described in the amino acid sequences set forth in SEQ ID Nos: 6, 7, and 8, respectively. In some cases, the light chain variable region comprises (i) an amino acid sequence having at least about 99% sequence identity to an amino acid sequence of SEQ ID NO: 5, and (ii) an L-CDR1, L-CDR2, and L-CDR3 as described in the amino acid sequences set forth in SEQ ID Nos: 6, 7, and 8, respectively.

Also provided and described herein are anti-human κ light chain antibodies and/or human κ light chain binding antibody fragments comprising:
a heavy chain variable region comprising an amino acid sequence having at least about 70%, at least about 80%, at least about 85%, at least about 90%, at least about 95%, at least about 96%, at least about 97%, at least about 98%, or at least about 99% identity to SEQ ID NO: 9; and/or
a light chain variable region comprising an amino acid sequence having at least about 70%, at least about 80%, at least about 85%, at least about 90%, at least about 95%, at least about 96%, at least about 97%, at least about 98%, or at least about 99% identity to SEQ ID NO: 13;
wherein the anti-human κ light chain antibodies and/or human κ light chain binding antibody fragments bind an epitope comprising SEQ ID NO: 33 (e.g., as measured by an ELISA).

In some cases, the heavy chain variable region comprises (i) an amino acid sequence having at least about 80% sequence identity to an amino acid sequence of SEQ ID NO: 9, and (ii) an H-CDR1, H-CDR2, and H-CDR3 as described in the amino acid sequences set forth in SEQ ID Nos: 10, 11, and 12, respectively. In some cases, the heavy chain variable region comprises (i) an amino acid sequence having at least about 85% sequence identity to an amino acid sequence of SEQ ID NO: 9, and (ii) an H-CDR1, H-CDR2, and H-CDR3 as described in the amino acid sequences set forth in SEQ ID Nos: 10, 11, and 12, respectively. In some cases, the heavy chain variable region comprises (i) an amino acid sequence having at least about 90% sequence identity to an amino acid sequence of SEQ ID NO: 9, and (ii) an H-CDR1, H-CDR2, and H-CDR3 as described in the amino acid sequences set forth in SEQ ID Nos: 10, 11, and 12, respectively. In some cases, the heavy chain variable region comprises (i) an amino acid sequence having at least about 95% sequence identity to an amino acid sequence of SEQ ID NO: 9, and (ii) an H-CDR1, H-CDR2, and H-CDR3 as described in the amino acid sequences set forth in SEQ ID Nos: 10, 11, and 12, respectively. In some cases, the heavy chain variable region comprises (i) an amino acid sequence having at least about 97% sequence identity to an amino acid sequence of SEQ ID NO: 9, and (ii) an H-CDR1, H-CDR2, and H-CDR3 as described in the amino acid sequences set forth in SEQ ID Nos: 10, 11, and 12, respectively. In some cases, the heavy chain variable region comprises (i) an amino acid sequence having at least about 99% sequence identity to an amino acid sequence of SEQ ID NO: 9, and (ii) an H-CDR1, H-CDR2, and H-CDR3 as described in the amino acid sequences set forth in SEQ ID Nos: 10, 11, and 12, respectively.

In some cases, the light chain variable region comprises (i) an amino acid sequence having at least about 80% sequence identity to an amino acid sequence of SEQ ID NO: 13, and (ii) an L-CDR1, L-CDR2, and L-CDR3 as described in the amino acid sequences set forth in SEQ ID Nos: 14, 15, and 16, respectively. In some cases, the light chain variable region comprises (i) an amino acid sequence having at least about 85% sequence identity to an amino acid sequence of SEQ ID NO: 13, and (ii) an L-CDR1, L-CDR2, and L-CDR3 as described in the amino acid sequences set forth in SEQ ID Nos: 14, 15, and 16, respectively. In some cases, the light chain variable region comprises (i) an amino acid sequence having at least about 90% sequence identity to an amino acid sequence of SEQ ID NO: 13, and (ii) an L-CDR1, L-CDR2, and L-CDR3 as described in the amino acid sequences set forth in SEQ ID Nos: 14, 15, and 16, respectively. In some cases, the light chain variable region comprises (i) an amino acid sequence having at least about 95% sequence identity to an amino acid sequence of SEQ ID NO: 13, and (ii) an L-CDR1, L-CDR2, and L-CDR3 as described in the amino acid sequences set forth in SEQ ID Nos: 14, 15, and 16, respectively. In some cases, the light chain variable region comprises (i) an amino acid sequence having at least about 97% sequence identity to an amino acid sequence of SEQ ID NO: 13, and (ii) an L-CDR1, L-CDR2, and L-CDR3 as described in the amino acid sequences set forth in SEQ ID Nos: 14, 15, and 16, respectively. In some cases, the light chain variable region comprises (i) an amino acid sequence having at least about 99% sequence identity to an amino acid sequence of SEQ ID NO: 13, and (ii) an L-CDR1, L-CDR2, and L-CDR3 as described in the amino acid sequences set forth in SEQ ID Nos: 14, 15, and 16, respectively.

Further provided and described herein are anti-human κ light chain antibodies and/or human κ light chain binding antibody fragments comprising:
a heavy chain variable region comprising an amino acid sequence having at least about 70%, at least about 80%, at least about 85%, at least about 90%, at least about 95%, at least about 96%, at least about 97%, at least about 98%, or at least about 99% identity to SEQ ID NO: 17; and/or
a light chain variable region comprising an amino acid sequence having at least about 70%, at least about 80%, at least about 85%, at least about 90%, at least about 95%, at least about 96%, at least about 97%, at least about 98%, or at least about 99% identity to SEQ ID NO: 21;
wherein the anti-human κ light chain antibodies and/or human κ light chain binding antibody fragments bind an epitope comprising SEQ ID NO: 33 (e.g., as measured by an ELISA).

In some cases, the heavy chain variable region comprises (i) an amino acid sequence having at least about 80% sequence identity to an amino acid sequence of SEQ ID NO: 17, and (ii) an H-CDR1, H-CDR2, and H-CDR3 as described in the amino acid sequences set forth in SEQ ID Nos: 18, 19, and 20, respectively. In some cases, the heavy chain variable region comprises (i) an amino acid sequence having at least about 85% sequence identity to an amino acid sequence of SEQ ID NO: 17, and (ii) an H-CDR1, H-CDR2, and H-CDR3 as described in the amino acid sequences set forth in SEQ ID Nos: 18, 19, and 20, respectively. In some cases, the heavy chain variable region comprises (i) an amino acid sequence having at least about 90% sequence identity to an amino acid sequence of SEQ ID NO: 17, and (ii) an H-CDR1, H-CDR2, and H-CDR3 as described in the amino acid sequences set forth in SEQ ID Nos: 18, 19, and 20, respectively. In some cases, the heavy chain variable region comprises (i) an amino acid sequence having at least about 95% sequence identity to an amino acid sequence of SEQ ID NO: 17, and (ii) an H-CDR1, H-CDR2, and H-CDR3 as described in the amino acid sequences set forth in SEQ ID Nos: 18, 19, and 20, respectively. In some cases, the heavy chain variable region comprises (i) an amino acid sequence having at least about 97% sequence identity to an amino acid sequence of SEQ ID NO: 17, and (ii) an H-CDR1, H-CDR2, and H-CDR3 as described in the amino acid sequences set forth in SEQ ID Nos: 18, 19, and 20, respectively. In some cases, the heavy chain variable region comprises (i) an amino acid sequence having at least about 99% sequence identity to an amino acid sequence of SEQ ID NO: 17, and (ii) an H-CDR1, H-CDR2, and H-CDR3 as described in the amino acid sequences set forth in SEQ ID Nos: 18, 19, and 20, respectively.

In some cases, the light chain variable region comprises (i) an amino acid sequence having at least about 80% sequence identity to an amino acid sequence of SEQ ID NO: 21, and (ii) an L-CDR1, L-CDR2, and L-CDR3 as described in the amino acid sequences set forth in SEQ ID Nos: 22, 23, and 24, respectively. In some cases, the light chain variable region comprises (i) an amino acid sequence having at least about 85% sequence identity to an amino acid sequence of SEQ ID NO: 21, and (ii) an L-CDR1, L-CDR2, and L-CDR3 as described in the amino acid sequences set forth in SEQ ID Nos: 22, 23, and 24, respectively. In some cases, the light chain variable region comprises (i) an amino acid sequence having at least about 90% sequence identity to an amino acid sequence of SEQ ID NO: 21, and (ii) an L-CDR1, L-CDR2, and L-CDR3 as described in the amino acid sequences set forth in SEQ ID Nos: 22, 23, and 24, respectively. In some cases, the light chain variable region comprises (i) an amino acid sequence having at least about 95% sequence identity to an amino acid sequence of SEQ ID NO: 21, and (ii) an L-CDR1, L-CDR2, and L-CDR3 as described in the amino acid sequences set forth in SEQ ID Nos: 22, 23, and 24, respectively. In some cases, the light chain variable region comprises (i) an amino acid sequence having at least about 97% sequence identity to an amino acid sequence of SEQ ID NO: 21, and (ii) an L-CDR1, L-CDR2, and L-CDR3 as described in the amino acid sequences set forth in SEQ ID Nos: 22, 23, and 24, respectively. In some cases, the light chain variable region comprises (i) an amino acid sequence having at least about 99% sequence identity to an amino acid sequence of SEQ ID NO: 21, and (ii) an L-CDR1, L-CDR2, and L-CDR3 as described in the amino acid sequences set forth in SEQ ID Nos: 22, 23, and 24, respectively.

Also provided and described herein are anti-human κ light chain antibodies and/or human κ light chain binding antibody fragments comprising:
a heavy chain variable region comprising an amino acid sequence having at least about 70%, at least about 80%, at least about 85%, at least about 90%, at least about 95%, at least about 96%, at least about 97%, at least about 98%, or at least about 99% identity to SEQ ID NO: 25; and/or
a light chain variable region comprising an amino acid sequence having at least about 70%, at least about 80%, at least about 85%, at least about 90%, at least about 95%, at least about 96%, at least about 97%, at least about 98%, or at least about 99% identity to SEQ ID NO: 29; wherein the anti-human κ light chain antibodies and/or human κ light chain binding antibody fragments bind an epitope comprising SEQ ID NO: 33 (e.g., as measured by an ELISA). In some embodiments, the heavy chain variable region comprises an amino acid sequence having at least about 80% identity to SEQ ID NO: 25, and the light chain variable region comprises an amino acid sequence having at least about 80% identity to SEQ ID NO: 29. In some embodiments, the heavy chain variable region comprises an amino acid sequence having at least about 85% identity to SEQ ID NO: 25, and the light chain variable region comprises an amino acid sequence having at least about 85% identity to SEQ ID NO: 29. In some embodiments, the heavy chain variable region comprises an amino acid sequence having at least about 90% identity to SEQ ID NO: 25, and the light chain variable region comprises an amino acid sequence having at least about 90% identity to SEQ ID NO: 29. In some embodiments, the heavy chain variable region comprises an amino acid sequence having at least about 95% identity to SEQ ID NO: 25, and the light chain variable region comprises an amino acid sequence having at least about 95% identity to SEQ ID NO: 29. In some embodiments, the heavy chain variable region comprises an amino acid sequence having at least about 97% identity to SEQ ID NO: 25, and the light chain variable region comprises an amino acid sequence having at least about 97% identity to SEQ ID NO: 29. In some embodiments, the heavy chain variable region comprises an amino acid sequence having at least about 98% identity to SEQ ID NO: 25, and the light chain variable region comprises an amino acid sequence having at least about 98% identity to SEQ ID NO: 29. In some embodiments, the heavy chain variable region comprises an amino acid sequence having at least about 99% sequence identity to SEQ ID NO: 25, and the light chain variable region comprises an amino acid sequence having at least about 99% sequence identity to SEQ ID NO: 29.

In some cases, the heavy chain variable region comprises (i) an amino acid sequence having at least about 80% sequence identity to an amino acid sequence of SEQ ID NO: 25, and (ii) an H-CDR1, H-CDR2, and H-CDR3 as described in the amino acid sequences set forth in SEQ ID Nos: 26, 27, and 28, respectively. In some cases, the heavy chain variable region comprises (i) an amino acid sequence having at least about 85% sequence identity to an amino acid sequence of SEQ ID NO: 25, and (ii) an H-CDR1, H-CDR2, and H-CDR3 as described in the amino acid sequences set forth in SEQ ID Nos: 26, 27, and 28, respectively. In some cases, the heavy chain variable region comprises (i) an amino acid sequence having at least about 90% sequence identity to an amino acid sequence of SEQ ID NO: 25, and (ii) an H-CDR1, H-CDR2, and H-CDR3 as described in the amino acid sequences set forth in SEQ ID Nos: 26, 27, and 28, respectively. In some cases, the heavy chain variable region comprises (i) an amino acid sequence having at least about 95% sequence identity to an amino acid sequence of SEQ ID NO: 25, and (ii) an H-CDR1, H-CDR2, and H-CDR3 as described in the amino acid sequences set forth in SEQ ID Nos: 26, 27, and 28, respectively. In some cases, the heavy chain variable region comprises (i) an amino acid sequence having at least about 97% sequence identity to an amino acid sequence of SEQ ID NO: 25, and (ii) an H-CDR1, H-CDR2, and H-CDR3 as described in the amino acid sequences set forth in SEQ ID Nos: 26, 27, and 28, respectively. In some cases, the heavy chain variable region comprises (i) an amino acid sequence having at least about 99% sequence identity to an amino acid sequence of SEQ ID NO: 25, and (ii) an H-CDR1, H-CDR2, and H-CDR3 as described in the amino acid sequences set forth in SEQ ID Nos: 26, 27, and 28, respectively.

In some cases, the light chain variable region comprises (i) an amino acid sequence having at least about 80% sequence identity to an amino acid sequence of SEQ ID NO: 29, and (ii) an L-CDR1, L-CDR2, and L-CDR3 as described in the amino acid sequences set forth in SEQ ID Nos: 30, 31, and 32, respectively. In some cases, the light chain variable region comprises (i) an amino acid sequence having at least about 85% sequence identity to an amino acid sequence of SEQ ID NO: 29, and (ii) an L-CDR1, L-CDR2, and L-CDR3 as described in the amino acid sequences set forth in SEQ ID Nos: 30, 31, and 32, respectively. In some cases, the light chain variable region comprises (i) an amino acid sequence having at least about 90% sequence identity to an amino acid sequence of SEQ ID NO: 29, and (ii) an L-CDR1, L-CDR2, and L-CDR3 as described in the amino acid sequences set forth in SEQ ID Nos: 30, 31, and 32, respectively. In some cases, the light chain variable region comprises (i) an amino acid sequence having at least about 95% sequence identity to an amino acid sequence of SEQ ID NO: 29, and (ii) an L-CDR1, L-CDR2, and L-CDR3 as described in the amino acid sequences set forth in SEQ ID Nos: 30, 31, and 32, respectively. In some cases, the light chain variable region comprises (i) an amino acid sequence having at least about 97% sequence identity to an amino acid sequence of SEQ ID NO: 29, and (ii) an L-CDR1, L-CDR2, and L-CDR3 as described in the amino acid sequences set forth in SEQ ID Nos: 30, 31, and 32, respectively. In some cases, the light chain variable region comprises (i) an amino acid sequence having at least about 99% sequence identity to an amino acid sequence of SEQ ID NO: 29, and (ii) an L-CDR1, L-CDR2, and L-CDR3 as described in the amino acid sequences set forth in SEQ ID Nos: 30, 31, and 32, respectively.

### Anti-Human κ Light Chain Constant Regions

In some embodiments, an anti-human κ light chain antibody encompasses intact or full-length antibodies, including antibodies of any class or sub-class, including IgG and sub-classes thereof, IgM, IgE, IgA, and IgD. In certain embodiments, the anti-human κ light chain antibodies comprise a human IgG constant region. In certain embodiments, the anti-human κ light chain antibodies comprise a human IgG1 constant region. In certain embodiments, the anti-human κ light chain antibodies comprise a human IgG2 constant region. In certain embodiments, the anti-human κ light chain antibodies comprise a human IgG3 constant region. In certain embodiments, the anti-human κ light chain antibodies comprise a human IgG4 constant region.

In some cases, the heavy chain sequence comprises (i) an amino acid sequence having at least about 80% sequence identity to an amino acid sequence of SEQ ID NO: 1, and (ii) an H-CDR1, H-CDR2, and H-CDR3 as described in the amino acid sequences set forth in SEQ ID Nos: 2, 3, and 4, respectively. In some cases, the heavy chain sequence comprises (i) an amino acid sequence having at least about 85% sequence identity to an amino acid sequence of SEQ ID NO: 1, and (ii) an H-CDR1, H-CDR2, and H-CDR3 as described in the amino acid sequences set forth in SEQ ID Nos: 2, 3, and 4, respectively. In some cases, the heavy chain sequence comprises (i) an amino acid sequence having at least about 90% sequence identity to an amino acid sequence of SEQ ID NO: 1, and (ii) an H-CDR1, H-CDR2, and H-CDR3 as described in the amino acid sequences set forth in SEQ ID Nos: 2, 3, and 4, respectively. In some cases, the heavy chain sequence comprises (i) an amino acid sequence having at least about 95% sequence identity to an amino acid sequence of SEQ ID NO: 1, and (ii) an H-CDR1, H-CDR2, and H-CDR3 as described in the amino acid sequences set forth in SEQ ID Nos: 2, 3, and 4, respectively. In some cases, the heavy chain sequence comprises (i) an amino acid sequence having at least about 97% sequence identity to an amino acid sequence of SEQ ID NO: 1, and (ii) an H-CDR1, H-CDR2, and H-CDR3 as described in the amino acid sequences set forth in SEQ ID Nos: 2, 3, and 4, respectively. In some cases, the heavy chain sequence comprises (i) an amino acid sequence having at least about 99% sequence identity to an amino acid sequence of SEQ ID NO: 1, and (ii) an H-CDR1, H-CDR2, and H-CDR3 as described in the amino acid sequences set forth in SEQ ID Nos: 2, 3, and 4, respectively.

In some cases, the heavy chain sequence comprises (i) an amino acid sequence having at least about 80% sequence identity to an amino acid sequence of SEQ ID NO: 9, and (ii) an H-CDR1, H-CDR2, and H-CDR3 as described in the amino acid sequences set forth in SEQ ID Nos: 10, 11, and 12, respectively. In some cases, the heavy chain sequence comprises (i) an amino acid sequence having at least about 85% sequence identity to an amino acid sequence of SEQ ID NO: 9, and (ii) an H-CDR1, H-CDR2, and H-CDR3 as described in the amino acid sequences set forth in SEQ ID Nos: 10, 11, and 12, respectively. In some cases, the heavy chain sequence comprises (i) an amino acid sequence having at least about 90% sequence identity to an amino acid sequence of SEQ ID NO: 9, and (ii) an H-CDR1, H-CDR2, and H-CDR3 as described in the amino acid sequences set forth in SEQ ID Nos: 10, 11, and 12, respectively. In some cases, the heavy chain sequence comprises (i) an amino acid sequence having at least about 95% sequence identity to an amino acid sequence of SEQ ID NO: 9, and (ii) an H-CDR1, H-CDR2, and H-CDR3 as described in the amino acid sequences set forth in SEQ ID Nos: 10, 11, and 12, respectively. In some cases, the heavy chain sequence comprises (i) an amino acid sequence having at least about 97% sequence identity to an amino acid sequence of SEQ ID NO: 9, and (ii) an H-CDR1, H-CDR2, and H-CDR3 as described in the amino acid sequences set forth in SEQ ID Nos: 10, 11, and 12, respectively. In some cases, the heavy chain sequence comprises (i) an amino acid sequence having at least about 99% sequence identity to an amino acid sequence of SEQ ID NO: 9, and (ii) an H-CDR1, H-CDR2, and H-CDR3 as described in the amino acid sequences set forth in SEQ ID Nos: 10, 11, and 12, respectively.

In some cases, the heavy chain sequence comprises (i) an amino acid sequence having at least about 80% sequence identity to an amino acid sequence of SEQ ID NO: 17, and (ii) an H-CDR1, H-CDR2, and H-CDR3 as described in the amino acid sequences set forth in SEQ ID Nos: 18, 19, and 20, respectively. In some cases, the heavy chain sequence comprises (i) an amino acid sequence having at least about 85% sequence identity to an amino acid sequence of SEQ ID NO: 17, and (ii) an H-CDR1, H-CDR2, and H-CDR3 as described in the amino acid sequences set forth in SEQ ID Nos: 18, 19, and 20, respectively. In some cases, the heavy chain sequence comprises (i) an amino acid sequence having at least about 90% sequence identity to an amino acid sequence of SEQ ID NO: 17, and (ii) an H-CDR1, H-CDR2, and H-CDR3 as described in the amino acid sequences set forth in SEQ ID Nos: 18, 19, and 20, respectively. In some cases, the heavy chain sequence comprises (i) an amino acid sequence having at least about 95% sequence identity to an amino acid sequence of SEQ ID NO: 17, and (ii) an H-CDR1, H-CDR2, and H-CDR3 as described in the amino acid sequences set forth in SEQ ID Nos: 18, 19, and 20, respectively. In some cases, the heavy chain sequence comprises (i) an amino acid sequence having at least about 97% sequence identity to an amino acid sequence of SEQ ID NO: 17, and (ii) an H-CDR1, H-CDR2, and H-CDR3 as described in the amino acid sequences set forth in SEQ ID Nos: 18, 19, and 20, respectively. In some cases, the heavy chain sequence comprises (i) an amino acid sequence having at least about 99% sequence identity to an amino acid sequence of SEQ ID NO: 17, and (ii) an H-CDR1, H-CDR2, and H-CDR3 as described in the amino acid sequences set forth in SEQ ID Nos: 18, 19, and 20, respectively.

In some cases, the heavy chain sequence comprises (i) an amino acid sequence having at least about 80% sequence identity to an amino acid sequence of SEQ ID NO: 25, and (ii) an H-CDR1, H-CDR2, and H-CDR3 as described in the amino acid sequences set forth in SEQ ID Nos: 26, 27, and 28, respectively. In some cases, the heavy chain sequence comprises (i) an amino acid sequence having at least about 85% sequence identity to an amino acid sequence of SEQ ID NO: 25, and (ii) an H-CDR1, H-CDR2, and H-CDR3 as described in the amino acid sequences set forth in SEQ ID Nos: 26, 27, and 28, respectively. In some cases, the heavy chain sequence comprises (i) an amino acid sequence having at least about 90% sequence identity to an amino acid sequence of SEQ ID NO: 25, and (ii) an H-CDR1, H-CDR2, and H-CDR3 as described in the amino acid sequences set forth in SEQ ID Nos: 26, 27, and 28, respectively. In some cases, the heavy chain sequence comprises (i) an amino acid sequence having at least about 95% sequence identity to an amino acid sequence of SEQ ID NO: 25, and (ii) an H-CDR1, H-CDR2, and H-CDR3 as described in the amino acid sequences set forth in SEQ ID Nos: 26, 27, and 28, respectively. In some cases, the heavy chain sequence comprises (i) an amino acid sequence having at least about 97% sequence identity to an amino acid sequence of SEQ ID NO: 25, and (ii) an H-CDR1, H-CDR2, and H-CDR3 as described in the amino acid sequences set forth in SEQ ID Nos: 26, 27, and 28, respectively. In some cases, the heavy chain sequence comprises (i) an amino acid sequence having at least about 99% sequence identity to an amino acid sequence of SEQ ID NO: 25, and (ii) an H-CDR1, H-CDR2, and H-CDR3 as described in the amino acid sequences set forth in SEQ ID Nos: 26, 27, and 28, respectively.

In some cases, the light chain sequence comprises (i) an amino acid sequence having at least about 80% sequence identity to an amino acid sequence of SEQ ID NO: 5, and (ii) an L-CDR1, L-CDR2, and L-CDR3 as described in the amino acid sequences set forth in SEQ ID Nos: 6, 7, and 8, respectively. In some cases, the light chain sequence comprises (i) an amino acid sequence having at least about 85% sequence identity to an amino acid sequence of SEQ ID NO: 5, and (ii) an L-CDR1, L-CDR2, and L-CDR3 as described in the amino acid sequences set forth in SEQ ID Nos: 6, 7, and 8, respectively. In some cases, the light chain sequence comprises (i) an amino acid sequence having at least about 90% sequence identity to an amino acid sequence of SEQ ID NO: 5, and (ii) an L-CDR1, L-CDR2, and L-CDR3 as described in the amino acid sequences set forth in SEQ ID Nos: 6, 7, and 8, respectively. In some cases, the light chain sequence comprises (i) an amino acid sequence having at least about 95% sequence identity to an amino acid sequence of SEQ ID NO: 5, and (ii) an L-CDR1, L-CDR2, and L-CDR3 as described in the amino acid sequences set forth in SEQ ID Nos: 6, 7, and 8, respectively. In some cases, the light chain sequence comprises (i) an amino acid sequence having at least about 97% sequence identity to an amino acid sequence of SEQ ID NO: 5, and (ii) an L-CDR1, L-CDR2, and L-CDR3 as described in the amino acid sequences set forth in SEQ ID Nos: 6, 7, and 8, respectively. In some cases, the light chain sequence comprises (i) an amino acid sequence having at least about 99% sequence identity to an amino acid sequence of SEQ ID NO: 5, and (ii) an L-CDR1, L-CDR2, and L-CDR3 as described in the amino acid sequences set forth in SEQ ID Nos: 6, 7, and 8, respectively.

In some cases, the light chain sequence comprises (i) an amino acid sequence having at least about 80% sequence identity to an amino acid sequence of SEQ ID NO: 13, and (ii) an L-CDR1, L-CDR2, and L-CDR3 as described in the amino acid sequences set forth in SEQ ID Nos: 14, 15, and 16, respectively. In some cases, the light chain sequence comprises (i) an amino acid sequence having at least about 85% sequence identity to an amino acid sequence of SEQ ID NO: 13, and (ii) an L-CDR1, L-CDR2, and L-CDR3 as described in the amino acid sequences set forth in SEQ ID Nos: 14, 15, and 16, respectively. In some cases, the light chain sequence comprises (i) an amino acid sequence having at least about 90% sequence identity to an amino acid sequence of SEQ ID NO: 13, and (ii) an L-CDR1, L-CDR2, and L-CDR3 as described in the amino acid sequences set forth in SEQ ID Nos: 14, 15, and 16, respectively. In some cases, the light chain sequence comprises (i) an amino acid sequence having at least about 95% sequence identity to an amino acid sequence of SEQ ID NO: 13, and (ii) an L-CDR1, L-CDR2, and L-CDR3 as described in the amino acid sequences set forth in SEQ ID Nos: 14, 15, and 16, respectively. In some cases, the light chain sequence comprises (i) an amino acid sequence having at least about 97% sequence identity to an amino acid sequence of SEQ ID NO: 13, and (ii) an L-CDR1, L-CDR2, and L-CDR3 as described in the amino acid sequences set forth in SEQ ID Nos: 14, 15, and 16, respectively. In some cases, the light chain sequence comprises (i) an amino acid sequence having at least about 99% sequence identity to an amino acid sequence of SEQ ID NO: 13, and (ii) an L-CDR1, L-CDR2, and L-CDR3 as described in the amino acid sequences set forth in SEQ ID Nos: 14, 15, and 16, respectively.

In some cases, the light chain sequence comprises (i) an amino acid sequence having at least about 80% sequence identity to an amino acid sequence of SEQ ID NO: 21, and (ii) an L-CDR1, L-CDR2, and L-CDR3 as described in the amino acid sequences set forth in SEQ ID Nos: 22, 23, and 24, respectively. In some cases, the light chain sequence comprises (i) an amino acid sequence having at least about 85% sequence identity to an amino acid sequence of SEQ ID NO: 21, and (ii) an L-CDR1, L-CDR2, and L-CDR3 as described in the amino acid sequences set forth in SEQ ID Nos: 22, 23, and 24, respectively. In some cases, the light chain sequence comprises (i) an amino acid sequence having at least about 90% sequence identity to an amino acid sequence of SEQ ID NO: 21, and (ii) an L-CDR1, L-CDR2, and L-CDR3 as described in the amino acid sequences set forth in SEQ ID Nos: 22, 23, and 24, respectively. In some cases, the light chain sequence comprises (i) an amino acid sequence having at least about 95% sequence identity to an amino acid sequence of SEQ ID NO: 21, and (ii) an L-CDR1, L-CDR2, and L-CDR3 as described in the amino acid sequences set forth in SEQ ID Nos: 22, 23, and 24, respectively. In some cases, the light chain sequence comprises (i) an amino acid sequence having at least about 97% sequence identity to an amino acid sequence of SEQ ID NO: 21, and (ii) an L-CDR1, L-CDR2, and L-CDR3 as described in the amino acid sequences set forth in SEQ ID Nos: 22, 23, and 24, respectively. In some cases, the light chain sequence comprises (i) an amino acid sequence having at least about 99% sequence identity to an amino acid sequence of SEQ ID NO: 21, and (ii) an L-CDR1, L-CDR2, and L-CDR3 as described in the amino acid sequences set forth in SEQ ID Nos: 22, 23, and 24, respectively.

In some cases, the light chain sequence comprises (i) an amino acid sequence having at least about 80% sequence identity to an amino acid sequence of SEQ ID NO: 29, and (ii) an L-CDR1, L-CDR2, and L-CDR3 as described in the amino acid sequences set forth in SEQ ID Nos: 30, 31, and 32, respectively. In some cases, the light chain sequence comprises (i) an amino acid sequence having at least about 85% sequence identity to an amino acid sequence of SEQ ID NO: 29, and (ii) an L-CDR1, L-CDR2, and L-CDR3 as described in the amino acid sequences set forth in SEQ ID Nos: 30, 31, and 32, respectively. In some cases, the light chain sequence comprises (i) an amino acid sequence having at least about 90% sequence identity to an amino acid sequence of SEQ ID NO: 29, and (ii) an L-CDR1, L-CDR2, and L-CDR3 as described in the amino acid sequences set forth in SEQ ID Nos: 30, 31, and 32, respectively. In some cases, the light chain sequence comprises (i) an amino acid sequence having at least about 95% sequence identity to an amino acid sequence of SEQ ID NO: 29, and (ii) an L-CDR1, L-CDR2, and L-CDR3 as described in the amino acid sequences set forth in SEQ ID Nos: 30, 31, and 32, respectively. In some cases, the light chain sequence comprises (i) an amino acid sequence having at least about 97% sequence identity to an amino acid sequence of SEQ ID NO: 29, and (ii) an L-CDR1, L-CDR2, and L-CDR3 as described in the amino acid sequences set forth in SEQ ID Nos: 30, 31, and 32, respectively. In some cases, the light chain sequence comprises (i) an amino acid sequence having at least about 99% sequence identity to an amino acid sequence of SEQ ID NO: 29, and (ii) an L-CDR1, L-CDR2, and L-CDR3 as described in the amino acid sequences set forth in SEQ ID Nos: 30, 31, and 32, respectively.

Described herein in certain embodiments are antibodies that bind misfolded human κ light chains. In certain embodiments, the antibodies bind aggregated human κ light chains. In certain embodiments, the antibodies bind alkylated human κ light chains. In certain embodiments, the antibodies bind amyloid human κ light chains. In certain embodiments, the antibodies bind aggregated human κ light chains with an equilibrium constant (K_{D}) of less than about 100 nanomolar. In certain embodiments, the antibodies bind aggregated human κ light chains with a K_{D} of less than about 90 nanomolar. In certain embodiments, the antibodies bind aggregated human κ light chains with a K_{D} of less than about 80 nanomolar. In certain embodiments, the antibodies bind aggregated human κ light chains with a K_{D} of less than about 70 nanomolar. In certain embodiments, the antibodies bind aggregated human κ light chains with a K_{D} of less than about 60 nanomolar. In certain embodiments, the antibodies bind aggregated human κ light chains with a K_{D} of less than about 50 nanomolar. In certain embodiments, the antibodies bind aggregated human κ light chains with a K_{D} of less than about 40 nanomolar. In certain embodiments, the antibodies bind aggregated human κ light chains with a K_{D} of less than about 30 nanomolar. In certain embodiments, the antibodies bind aggregated human κ light chains with a K_{D} of less than about 20 nanomolar. In certain embodiments, the antibodies bind aggregated human κ light chains with a K_{D} of less than about 15 nanomolar. In certain embodiments, the antibodies bind aggregated human κ light chains with a K_{D} of less than about 10 nanomolar. In certain embodiments, the antibodies bind aggregated human κ light chains with a K_{D} of less than about 5 nanomolar. In certain embodiments, the antibodies bind aggregated human κ light chains with a K_{D} of greater than about 0.01, 0.05, 0.1, 0.5, 1, 10, 20, 30, 40, or 50 nanomolar. In certain embodiments, the antibodies bind amyloid human κ light chains with a K_{D} of less than about 100 nanomolar. In certain embodiments, the antibodies bind amyloid human κ light chains with a K_{D} of less than about no more than about 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1.5 nM, 1.2 nM, 1 nM, 0.9 nM, 0.8 nM, 0.7 nM, 0.6 nM, 0.5 nM, 0.1 nM, 0.01 nM, or less. In certain embodiments, the antibodies bind amyloid human κ light chains with a K_{D} of less than about 80 nanomolar. In certain embodiments, the antibodies bind amyloid human κ light chains with a K_{D} of less than about 70 nanomolar. In certain embodiments, the antibodies bind amyloid human κ light chains with a K_{D} of less than about 60 nanomolar. In certain embodiments, the antibodies bind amyloid human κ light chains with a K_{D} of less than about 50 nanomolar. In certain embodiments, the antibodies bind amyloid human κ light chains with a K_{D} of less than about 40 nanomolar. In certain embodiments, the antibodies bind amyloid human κ light chains with a K_{D} of less than about 30 nanomolar. In certain embodiments, the antibodies bind amyloid human κ light chains with a K_{D} of less than about 20 nanomolar. In certain embodiments, the antibodies bind amyloid human κ light chains with a K_{D} of less than about 15 nanomolar. In certain embodiments, the antibodies bind amyloid human κ light chains with a K_{D} of less than about 10 nanomolar. In certain embodiments, the antibodies bind amyloid human κ light chains with a K_{D} of greater than about 0.01 nM, 0.1 nM, 0.5 nM, 0.6 nM, 0.7 nM, 0.8 nM, 0.9 nM, 1 nM, 1.2 nM, 1.5 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, or more. In certain embodiments, the antibodies bind amyloid human κ light chains with a K_{D} of about 50, 40, 30, 20, 15, 10, or 5 nanomolar. In certain embodiments, the antibodies bind alkylated human κ light chains with a K_{D} of less than about 70 nanomolar. In certain embodiments, the antibodies bind alkylated human κ light chains with a K_{D} of less than about 60 nanomolar. In certain embodiments, the antibodies bind alkylated human κ light chains with a K_{D} of less than about 50 nanomolar. In certain embodiments, the antibodies bind alkylated human κ light chains with a K_{D} of less than about 40 nanomolar. In certain embodiments, the antibodies bind alkylated human κ light chains with a K_{D} of less than about 30 nanomolar. In certain embodiments, the antibodies bind alkylated human κ light chains with a K_{D} of less than about 20 nanomolar. In certain embodiments, the antibodies bind alkylated human κ light chains with a K_{D} of less than about 15 nanomolar. In certain embodiments, the antibodies bind alkylated human κ light chains with a K_{D} of less than about 10 nanomolar. In certain embodiments, the antibodies bind alkylated human κ light chains with a K_{D} of less than about 5 nanomolar. In certain embodiments, the antibodies bind alkylated human κ light chains with a K_{D} of greater than about 0.01, 0.05, 0.1, 0.5, 1, 10, 20, 30, 40, or 50 nanomolar. In some embodiments, the K_{D} is measured by an ELISA or biolayer interferometry. In some embodiments, the K_{D} is measured by an ELISA. In some embodiments, the K_{D} is measured by biolayer interferometry.

In some embodiments, the anti-human kappa light chain antibodies or human kappa light chain binding fragments thereof are characterized by a higher binding affinity for a misfolded kappa light chain than a native kappa light chain. In some embodiments, the anti-human kappa light chain antibodies or human kappa light chain binding fragments thereof bind to a misfolded kappa light chain with an equilibrium constant (K_{D}) of no more than about 50%, 40%, 30%, 20%, 10%, 5%, 3%, 2%, 1%, 0.1%, 0.01%, or less of a K_{D} of the anti-human kappa light chain antibodies or human kappa light chain binding fragments thereof binding to a native kappa light chain. In some embodiments, the anti-human kappa light chain antibodies or human kappa light chain binding fragments thereof bind to a misfolded kappa light chain having an equilibrium constant (K_{D}) of greater than about 1%, 3%, 5%, 10%, 15%, 20%, 30%, 40%, or 50% of a K_{D} of the anti-human kappa light chain antibodies or human kappa light chain binding fragments thereof binding to a native kappa light chain. In some embodiments, the anti-human kappa light chain antibodies or human kappa light chain binding fragments thereof bind to a misfolded kappa light chain having an equilibrium constant (K_{D}) that is about 3%, 5%, 10%, 15%, 20%, 25%, or 30% of a K_{D} of the anti-human kappa light chain antibodies or human kappa light chain binding fragments thereof binding to a native kappa light chain.

In some embodiments, the anti-human kappa light chain antibodies or human kappa light chain binding fragments thereof are characterized by a higher binding affinity for a misfolded kappa amyloid than a native kappa light chain. In some embodiments, the anti-human kappa light chain antibodies or human kappa light chain binding fragments thereof bind to a misfolded kappa amyloid having an equilibrium constant (K_{D}) of no more than about 50%, 40%, 30%, 20%, 10%, 5%, or 3% of a K_{D} of the anti-human kappa light chain antibodies or human kappa light chain binding fragments thereof binding to a native kappa light chain. In some embodiments, the anti-human kappa light chain antibodies or human kappa light chain binding fragments thereof bind to a misfolded kappa amyloid having an equilibrium constant (K_{D}) of greater than about 1%, 3%, 5%, 10%, 15%, 20%, 30%, 40%, or 50% of a K_{D} of the anti-human kappa light chain antibodies or human kappa light chain binding fragments thereof binding to a native kappa light chain. In some embodiments, the anti-human kappa light chain antibodies or human kappa light chain binding fragments thereof bind to a misfolded kappa amyloid having an equilibrium constant (K_{D}) that is about 3%, 5%, 10%, 15%, 20%, 25%, or 30% of a K_{D} of the anti-human kappa light chain antibodies or human kappa light chain binding fragments thereof binding to a native kappa light chain.

In some embodiments, the anti-human kappa light chain antibodies or human kappa light chain binding fragments thereof are characterized by a higher binding affinity for a misfolded kappa aggregate than a native kappa light chain. In some embodiments, the anti-human kappa light chain antibodies or human kappa light chain binding fragments thereof bind to a misfolded kappa aggregate (e.g., aggregate of misfolded κ light chain) having an equilibrium constant (K_{D}) of no more than about 50%, 40%, 30%, 20%, 10%, 5%, or 3% of a K_{D} of the anti-human kappa light chain antibodies or human kappa light chain binding fragments thereof binding to a native kappa light chain. In some embodiments, the anti-human kappa light chain antibodies or human kappa light chain binding fragments thereof bind to a misfolded kappa aggregate having an equilibrium constant (K_{D}) of greater than about 1%, 3%, 5%, 10%, 15%, 20%, 30%, 40%, or 50% of a K_{D} of the anti-human kappa light chain antibodies or human kappa light chain binding fragments thereof binding to a native kappa light chain. In some embodiments, the anti-human kappa light chain antibodies or human kappa light chain binding fragments thereof bind to a misfolded kappa aggregate having an equilibrium constant (K_{D}) that is about 3%, 5%, 10%, 15%, 20%, 25%, or 30% of a K_{D} of the anti-human kappa light chain antibodies or human kappa light chain binding fragments thereof binding to a native kappa light chain.

In some embodiments, the anti-human kappa light chain antibodies or human kappa light chain binding fragments thereof are characterized by a higher binding affinity for an alkylated kappa light chain than a native kappa light chain. In some embodiments, the anti-human kappa light chain antibodies or human kappa light chain binding fragments thereof bind to an alkylated kappa light chain having an equilibrium constant (K_{D}) of no more than about 50%, 40%, 30%, 20%, 10%, 5%, or 3% of a K_{D} of the anti-human kappa light chain antibodies or human kappa light chain binding fragments thereof binding to a native kappa light chain. In some embodiments, the anti-human kappa light chain antibodies or human kappa light chain binding fragments thereof bind to an alkylated kappa light chain having an equilibrium constant (K_{D}) of greater than about 1%, 3%, 5%, 10%, 15%, 20%, 30%, 40%, or 50% of a K_{D} of the anti-human kappa light chain antibodies or human kappa light chain binding fragments thereof binding to a native kappa light chain. In some embodiments, the anti-human kappa light chain antibodies or human kappa light chain binding fragments thereof bind to an alkylated kappa light chain having an equilibrium constant (K_{D}) of about 3%, 5%, 10%, 15%, 20%, 25%, or 30% of a K_{D} of the anti-human kappa light chain antibodies or human kappa light chain binding fragments thereof binding to a native kappa light chains.

In some embodiments, a K_{D} of the anti-human kappa light chain antibodies or human kappa light chain binding fragments thereof binding to a misfolded kappa light chain, a misfolded kappa amyloid, a kappa light chain aggregate, an alkylated kappa light chain, or a native kappa light chain can be measured by an ELISA. In some embodiments, a K_{D} of the anti-human kappa light chain antibodies or human kappa light chain binding fragments thereof binding to a misfolded kappa light chain or a native kappa light chain can be measured by an ELISA or biolayer interferometry. In some embodiments, a K_{D} of the anti-human kappa light chain antibodies or human kappa light chain binding fragments thereof binding to a misfolded kappa amyloid or a native kappa light chain can be measured by an ELISA or biolayer interferometry. In some embodiments, a K_{D} of the anti-human kappa light chain antibodies or human kappa light chain binding fragments thereof binding to a kappa light chain aggregate or a native kappa light chain can be measured by an ELISA or biolayer interferometry. In some embodiments, a K_{D} of the anti-human kappa light chain antibodies or human kappa light chain binding fragments thereof binding to an alkylated kappa light chain or a native kappa light chain can be measured by an ELISA or biolayer interferometry.

In some embodiments, the anti-κ light chain antibody or κ light chain binding fragment thereof can reduce a level of, or prevent accumulation of, a polypeptide, a fibril, or a polypeptide aggregate, such as in a subject, a cell, or a sample. In some embodiments, the anti-κ light chain antibody or κ light chain binding fragment thereof can reduce or prevent accumulation of a misfolded polypeptide, including a misfolded κ light chain, a misfolded κ light chain aggregate, or a misfolded κ light chain fibril.

In some embodiments, the anti-κ light chain antibody or κ light chain binding fragment thereof can have a half-maximal inhibitory concentration (IC₅₀) of no more than about 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1.5 nM, 1.2 nM, 1 nM, 0.9 nM, 0.8 nM, 0.7 nM, 0.6 nM, 0.5 nM, 0.1 nM, 0.01 nM, or less for reducing an amount of a polypeptide (e.g., a misfolded κ light chain), a fibril (e.g., a κ light chain fibril), or a polypeptide aggregate (e.g., a κ light chain aggregate).

In some embodiments, the anti-κ light chain antibody or κ light chain binding fragment thereof can have a half-maximal inhibitory concentration (IC₅₀) of at least about about 0.01 nM, 0.1 nM, 0.5 nM, 0.6 nM, 0.7 nM, 0.8 nM, 0.9 nM, 1 nM, 1.2 nM, 1.5 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 150 nM, 200 nM, 250 nM 300 nM, 400 nM, or higher for reducing an amount of a polypeptide (e.g., a misfolded κ light chain), a fibril (e.g., a κ light chain fibril), or a polypeptide aggregate (e.g., a κ light chain aggregate).

In some embodiments, the antibodies that bind misfolded human κ light chains are thermally stable. In some embodiments, the antibodies that bind misfolded human κ light chains comprise a melting temperature (Tₘ). In some embodiments, the antibodies comprise a Tₘ determined by an inflection point of protein fluorescence or protein structural stability. In some embodiments, the antibodies can comprise a Tₘ that is at least about 50 °C, 60 °C, 70 °C, 80 °C, or 90 °C In some embodiments, the antibodies can comprise a Tₘ that is at least about 70 °C In some embodiments, the antibodies can comprise a Tₘ that is at least about 80 °C In some embodiments, the antibodies can comprise a Tₘ that is at least about 90 °C In some embodiments, the antibodies can comprise a Tₘ that is from about 50 °C to about 95 °C In some embodiments, the antibodies can comprise a Tₘ that is from about 60 °C to about 95 °C In some embodiments, the antibodies can comprise a Tₘ that is from about 70 °C to about 95 °C In some embodiments, the antibodies can comprise a Tₘ that is from about 75 °C to about 95 °C In some embodiments, the antibodies can comprise a Tₘ that is about 75 °C In some embodiments, the antibodies can comprise a Tₘ that is about 80 °C In some embodiments, the antibodies can comprise a Tₘ that is about 85 °C In some embodiments, the antibodies can comprise a Tₘ that is about 90 °C

In some embodiments, the Tₘ can be measured by a thermal shift assay or a thermal stability assay. In some embodiments, the Tₘ can be determined by fluorescence of the antibody. In some embodiments, the Tₘ can be determined by a ratio of the fluorescence of the antibody is measured at 350 nm and 330 nm.

In some embodiments, the anti-human κ light chain antibodies and/or human κ light chain binding antibody fragments can comprise a Fab, a Fab′, a F(ab′)₂, a scFv, a dsFv, a ds-scFv, a dimer, a minibody, or a diabody.

In some embodiments, the anti-human κ light chain antibodies and/or human κ light chain binding antibody fragments provided herein can be phagocytosed by a cell.

In some embodiments, the cell can be a neutrophil, a macrophage, a monocyte, a dendritic cell, or a mast cell. In some embodiments, the cell can be a cell obtained from a subject. In some embodiments, the cell can be from a monocytic line. In some embodiments, the cell can be a THP-1 monocyte cell line. In some embodiments, the anti-human κ light chain antibodies and/or binding antibody fragments can be phagocytosed by a cell (e.g., a phagocytic cell, THP-1 monocyte) at an effective concentration (EC₅₀) of about at least about 0.01 nM, 0.1 nM, 0.2 nM, 0.3 nM, 0.4 nM, 0.5 nM, 0.6 nM, 0.7 nM, 0.8 nM, 0.9 nM, 1 nM, 1.2 nM, 1.5 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 150 nM, 200 nM, 250 nM 300 nM, 400 nM, or more than a control (e.g., isotype control or Fc receptor null control) after a period of about 2 hours in contact with a monocyte. In some embodiments, the anti-human κ light chain antibodies and/or binding antibody fragments can be phagocytosed by a cell (e.g., a phagocytic cell, THP-1 monocyte) at an effective concentration (EC₅₀) of no more than about 0.2 nM, 0.3 nM, 0.4 nM, 0.5 nM, or 0.6 nM, 0.8 nM, 0.9 nM, 1 nM, 1.2 nM, 1.5 nM, 2 nM, 3 nM, 4 nM, 5 nM, 6 nM, 7 nM, 8 nM, 9 nM, 10 nM, 20 nM, 30 nM, 40 nM, 50 nM, or less than a control (e.g., isotype control or Fc receptor null control) after a period of about 2 hours in contact with a monocyte. In some embodiments, the anti-human κ light chain antibodies and/or binding antibody fragments can be phagocytosed by a cell (e.g., a phagocytic cell, THP-1 monocyte) at an effective concentration (EC₅₀) of about at least about 0.01 nM, 0.1 nM, 0.2 nM, 0.3 nM, 0.5 nM, 0.6 nM, 0.7 nM, 0.8 nM, 0.9 nM, 1 nM, 1.2 nM, 1.5 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 150 nM, 200 nM, 250 nM 300 nM, 400 nM, or more than a control (e.g., isotype control or Fc receptor null control) after a period of about 24 hours in contact with a monocyte. In some embodiments, the anti-human κ light chain antibodies and/or binding antibody fragments can be phagocytosed by a cell (e.g., a phagocytic cell, THP-1 monocyte) at an effective concentration (EC₅₀) of no more than about 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1.5 nM, 1.2 nM, 1 nM, 0.9 nM, 0.8 nM, 0.7 nM, 0.6 nM, 0.5 nM, 0.4 nM, 0.3 nM, 0.2 nM, 0.1 nM, 0.05 nM, 0.01 nM, or less than a control (e.g., isotype control or Fc receptor null control) after a period of about 24 hours in contact with the cell (e.g., a monocyte, THP-1 monocyte).

TABLE 1
Sequences

| SEQ ID | | |
|---|---|---|
| NO: | SEQUENCE | ANNOTATION |
| 1 | EVQLLESGGGLVQPGGSLRLSCAASGFTFSSYGM | AB1_HV |
| | SWVRQAPGKGLEWVSAISYSGGSTYYADSVKGR | |
| | FTISRDNSKNTLYLQMNSLRAEDTAVYYCASTGW | |
| | ESDAFDIWGQGTLVTVSS | |
| 2 | AASGFTFSSYGMS | AB1_H-CDR1 |
| 3 | AISYSGGSTY | AB1_H-CDR2 |
| 4 | ASTGWESDAFDI | AB1_H-CDR3 |
| 5 | DIQMTQSPSSLSASVGDRVTITCRASQGIRNDLGW | AB1_LV |
| | YQQKPGKAPKLLIYAASSLQTGVPSRFSGSGSGTH | |
| | FTLTISSLQPDDFATYYCQQGNSFPPTFGQGTKVEI | |
| | K | |
| 6 | RASQGIRNDLG | AB1_L-CDR1 |
| 7 | YAASSLQT | AB1_L-CDR2 |
| 8 | QQGNSFPPT | AB1_L-CDR3 |
| 9 | QVQLVQSGAEVKKPGSSVKVSCKASGYTFTSLWI | AB4_HV |
| | QWVRQAPGQGLEWMGSIYPGDGDTQYIQKFRGR | |
| | VTITADESTSTAYMELSSLRSEDTAVYYCSIITTAPD | |
| | YWGQGTLVTVSS | |
| 10 | KASGYTFTSLWIQ | AB4_H-CDR1 |
| 11 | SIYPGDGDTQ | AB4_H-CDR2 |
| 12 | SIITTAPDY | AB4_H-CDR3 |
| 13 | DVVMTQSPLSLPVTLGQPASISCKSSQDLLDVDGKTY | AB4_LV |
| | LNWFQQRPGQSPRRLIYHVSKLDSGVPDRFSGSGSGT | |
| | DFTLKISRVEAEDVGVYYCHQGTHFPQTFGGGTKVEI | |
| | K | |
| 14 | KSSQDLLDVDGKTYLN | AB4_L-CDR1 |
| 15 | YHVSKLDS | AB4_L-CDR2 |
| 16 | HQGTHFPQT | AB4_L-CDR3 |
| 17 | QVQLVQSGAEVKKPGSSVKVSCKASGYTFRSQWI | AB2_HV |
| | QWVRQAPGQGLEWMGSIYPGDGDTQYIQKFRGR | |
| | VTITADESTSTAYMELSSLRSEDTAVYYCSIITTAPD | |
| | YWGQGTLVTVSS | |
| 18 | KASGYTFRSQWIQ | AB2_H-CDR1 |
| 19 | SIYPGDGDTQ | AB2_H-CDR2 |
| 20 | SIITTAPDY | AB2_H-CDR3 |
| 21 | DVVMTQSPLSLPVTLGQPASISCKSSQDLLEGDGK | AB2_LV |
| | TYLNWFQQRPGQSPRRLIYHVSKLDSGVPDRFSG | |
| | SGSGTDFTLKISRVEAEDVGVYYCHQGTHFPQTF | |
| | GGGTKVEIK | |
| 22 | KSSQDLLEGDGKTYLN | AB2_L-CDR1 |
| 23 | YHVSKLDS | AB2_L-CDR2 |
| 24 | HQGTHFPQT | AB2_L-CDR3 |
| 25 | QVQLVQSGAEVKKPGSSVKVSCKASGYTFRSKWI | AB3_HV |
| | QWVRQAPGQGLEWMGSIYPGDGDTQYIQKFRGR | |
| | VTITADESTSTAYMELSSLRSEDTAVYYCSIITTAPD | |
| | YWGQGTLVTVSS | |
| 26 | KASGYTFRSKWIQ | AB3_H-CDR1 |
| 27 | SIYPGDGDTQ | AB3_H-CDR2 |
| 28 | SIITTAPDY | AB3_H-CDR3 |
| 29 | DVVMTQSPLSLPVTLGQPASISCKSSQDLLEGDGK | AB3_LV |
| | TYLNWFQQRPGQSPRRLIYHVSKLDSGVPDRFSG | |
| | SGSGTDFTLKISRVEAEDVGVYYCHQGTHFPQTF | |
| | GGGTKVEIK | |
| 30 | KSSQDLLEGDGKTYLN | AB3_L-CDR1 |
| 31 | YHVSKLDS | AB3_L-CDR2 |
| 32 | HQGTHFPQT | AB3_L-CDR3 |
| 33 | STYSLSSTLT | Misfolded K LC |
| | | epitope |

Provided herein are compositions (e.g., pharmaceutical compositions) comprising the anti-human κ light chain antibodies and/or human κ light chain binding antibody fragments described herein. A composition or pharmaceutical formulation or pharmaceutical composition generally encompasses and/or refers to a preparation which is in such form as to permit the biological activity of an active ingredient contained therein to be effective, and which contains no additional components which are unacceptably toxic to a subject to which the formulation would be administered. Pharmaceutical formulations are generally sterile (e.g., aseptic or free from all living microorganisms and their spores). In some embodiments, a composition or pharmaceutical composition comprises at least one, at least two, at least three, or at least four, or any combination of the anti-human κ light chain antibodies and/or human κ light chain binding antibody fragments described herein. In some embodiments, a composition or pharmaceutical composition comprises one or more of the anti-human κ light chain antibodies and/or human κ light chain binding antibody fragments described herein.

Compositions comprising the anti-human κ light chain antibodies and/or human κ light chain binding antibody fragments described herein can include in a pharmaceutical composition comprising one or more pharmaceutically acceptable excipients, carriers, and diluents. In certain embodiments, the antibodies of the current disclosure are administered suspended in a sterile and/or isotonic solution. In certain embodiments, the solution comprises about 0.9% NaCl. In certain embodiments, the solution comprises about 5.0% dextrose. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, L-Histidine/L-histidine monohydrochloride monohydrate, trehalose, and dextran 40; amino acids, for example, glycine or arginine; antioxidants, for example, ascorbic acid, methionine; or chelating agents, for example, EDTA or EGTA.

Formulations for administration of antibodies can comprise one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine or arginine; antioxidants, for example, ascorbic acid, methionine; or chelating agents, for example, EDTA or EGTA. Additionally, a compound or molecule that relieves pain at the injection site can be included, such as hyaluronidase, for example at a concentration of from about 2,000 U/ml to about 12,000 U/ml.

In certain embodiments, the anti-human κ light chain antibodies or human κ light chain binding antibody fragments of described herein are shipped/stored lyophilized and reconstituted before administration. In certain embodiments, lyophilized antibody formulations comprise a bulking agent such as, mannitol, sorbitol, sucrose, trehalose, dextran 40, or combinations thereof. The lyophilized formulation can be contained in a vial comprised of glass or other suitable non-reactive material. The antibodies when formulated, whether reconstituted or not, can be buffered at a certain pH, generally less than 7.0. In certain embodiments, the pH can be between 4.5 and 6.5, 4.5 and 6.0, 4.5 and 5.5, 4.5 and 5.0, or 5.0 and 6.0.

In some embodiments, the anti-human κ light chain antibodies and/or human κ light chain binding antibody fragments can be configured for parenteral, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural, or intrasternal injection. In some embodiments, the compositions comprising the anti-human κ light chain antibodies and/or human κ light chain binding antibody fragments can be configured for intravenous injection.

In some cases, the composition comprising the anti-human κ light chain antibodies and/or human κ light chain binding antibody fragments can be configured for delivery to a subject. In some cases, the subject can be a mammal. In some cases, the subject can be a primate. In some cases, the subject can be a human. In some cases, the subject can be a non-human primate. In some embodiments, the subject can have, or can be suspected of having, a disease or condition characterized by kappa light chain aggregates. In some embodiments, the subject can have, or can be suspected of having, a disease or condition characterized by expression of disease-associated immunoglobulin light chain sequences arising from VJ recombination events involving Immunoglobulin Kappa Variable 1 (IGKV1) germline v-gene. In some embodiments, the subject can have, or can be suspected of having, a disease or condition characterized by expression of disease-associated immunoglobulin light chain sequences arising from VJ recombination events involving Immunoglobulin Kappa Variable 4 (IGKV4) germline v-gene. In some cases, the disease or condition characterized by kappa light chain aggregates can be a plasma cell disorder, systemic amyloidosis, or light chain amyloidosis. In some cases, the disease or condition characterized by kappa light chain aggregates can be an amyloidosis. In some embodiments, the disease or condition characterized by kappa light chain aggregates can be a systemic amyloidosis. In some embodiments, the disease or condition characterized by kappa light chain aggregates can be a renal amyloidosis, a cardiac amyloidosis, a gastrointestinal amyloidosis, or a neuro-amyloidosis. In some embodiments, the disease or condition characterized by kappa light chain aggregates can be a renal amyloidosis. In some embodiments, the disease or condition characterized by kappa light chain aggregates can be a cardiac amyloidosis. In some embodiments, the disease or condition characterized by kappa light chain aggregates can be a gastrointestinal amyloidosis. In some embodiments, the disease or condition characterized by kappa light chain aggregates can be a neuro-amyloidosis. In some embodiments, the disease or condition characterized by kappa light chain aggregates can be a muscular amyloidosis. In some embodiments, the disease or condition characterized by kappa light chain aggregates can be a light chain amyloidosis linked to expression of disease-associated immunoglobulin light chain sequences arising from VJ recombination events involving Immunoglobulin Kappa Variable 4 (IGKV4) germline v-gene. In some embodiments, the disease or condition characterized by kappa light chain aggregates can be a light chain amyloidosis linked to expression of disease-associated immunoglobulin light chain sequences arising from VJ recombination events involving Immunoglobulin Kappa Variable 1 (IGKV1) germline v-gene. In certain embodiments, the disease or condition characterized by kappa light chain aggregates can be a light chain amyloidosis. In some embodiments, the disease or condition characterized by kappa light chain aggregates can be a plasma cell dyscrasias. In some cases, the plasma cell dyscrasia can be amyloidosis, monoclonal gammopathy of unknown significance (MGUS), multiple myeloma (MM), plasma cell leukemia (PCL), or any combination thereof. In some embodiments, the amyloidosis or plasma cell dyscrasia can be a systemic amyloidosis. In some embodiments, the amyloidosis or plasma cell dyscrasia can be a light chain amyloidosis. In some embodiments, the amyloidosis or plasma cell dyscrasia can be a cardiac amyloidosis. In some embodiments, amyloidosis or plasma cell dyscrasia can be a renal amyloidosis.

Also described herein are kits comprising one or more of the antibodies described herein in a suitable container and one or more additional components selected from: instructions for use; a diluent, an excipient, a carrier, and a device for administration.

In certain embodiments, described herein is a method of preparing a treatment comprising admixing one or more pharmaceutically acceptable excipients, carriers, or diluents and the anti-human κ light chain antibodies and/or human κ light chain binding antibody fragments. In certain embodiments, described herein is a method of preparing a cancer treatment for storage or shipping comprising lyophilizing one or more antibodies of the current disclosure.

### Cells and Nucleic Acids

Cells, cell lines, and/or cell cultures are used interchangeably, and generally include and/or refer to cells into which exogenous nucleic acid has been introduced, including the progeny of such cells. Cells include transformants and transformed cells, which include the primary transformed cell and progeny derived therefrom without regard to the number of passages. In some embodiments, cells can be eukaryotic cells. In some embodiments, cells can be prokaryotic cells. In some embodiments, cells can be mammalian cells. In some embodiments, cells can be a Chines Hamster Ovary (CHO) cell, a mouse myeloma (MSO) cell, or a human embryonic kidney cell (e.g., HEK293 cell). In some embodiments, a cell comprises a nucleic acid encoding any of the anti-human kappa light chain antibodies provided in this disclosure or binding fragments thereof. In some embodiments, a cell comprises a nucleic acid encoding a heavy chain variable region and/or a light chain variable region of any of the anti-human kappa light chain antibodies provided in this disclosure or binding fragments thereof.

An isolated nucleic acid generally encompasses and/or refers to a nucleic acid molecule that has been separated from a component of its natural environment. An isolated nucleic acid encompasses a nucleic acid molecule contained in cells that ordinarily contain the nucleic acid molecule, but the nucleic acid molecule is present extrachromosomally or at a chromosomal location that is different from its natural chromosomal location. An isolated nucleic acid encoding an antibody encompasses and/or refers to one or more nucleic acid molecules encoding antibody heavy and/or light chains (or fragments thereof), including such nucleic acid molecule(s) in a single vector or separate vectors, and such nucleic acid molecule(s) present at one or more locations in a host cell. In some embodiments, provided herein is a nucleic acid sequence encoding the anti-human κ light chain antibodies and/or human κ light chain binding antibody fragments described herein. In some embodiments, the nucleic acid sequence encodes a heavy chain variable region and/or a light chain variable region of the anti-human κ light chain antibodies and/or human κ light chain binding antibody fragments described herein. In some embodiments, also provided is a host cell comprising the nucleic acid sequence encoding the anti-human κ light chain antibodies and/or human κ light chain binding antibody fragments. In some embodiments, the nucleic acid sequence can encode an amino acid sequence comprising at least about 60%, at least about 70%, at least about 80%, at least about 85%, at least about 90%, at least about 95%, at least about 96%, at least about 97%, at least about 98%, or at least about 99% sequence identity to any one of SEQ ID NOs: 1, 5, 9, 13, 17, 21, 25, and 29.

In some embodiments, the nucleic acid sequence can encode an amino acid sequence comprising at least 70% sequence identity to any one of SEQ ID NOs: 1, 5, 9, 13, 17, 21, 25, and 29. In some embodiments, the nucleic acid sequence can encode an amino acid sequence comprising at least 80% sequence identity to any one of SEQ ID NOs: 1, 5, 9, 13, 17, 21, 25, and 29. In some embodiments, the nucleic acid sequence can encode an amino acid sequence comprising at least 90% sequence identity to any one of SEQ ID NOs: 1, 5, 9, 13, 17, 21, 25, and 29. In some embodiments, the nucleic acid sequence can encode an amino acid sequence comprising at least 95% sequence identity to any one of SEQ ID NOs: 1, 5, 9, 13, 17, 21, 25, and 29. In some embodiments, the nucleic acid sequence can encode an amino acid sequence comprising any one of SEQ ID NOs: 1, 5, 9, 13, 17, 21, 25, and 29.

In some embodiments, the nucleic acid sequence can encode an amino acid sequence comprising at least about 60%, at least about 70%, at least about 80%, at least about 85%, at least about 90%, at least about 95%, at least about 96%, at least about 97%, at least about 98%, or at least about 99% sequence identity to any one pair of SEQ ID NOs: 1 and 5, 9 and 13, 17 and 21, or 25 and 29. In some embodiments, the nucleic acid sequence can encode an amino acid sequence comprising at least 70% sequence identity to any one pair of SEQ ID NOs: 1 and 5, 9 and 13, 17 and 21, or 25 and 29. In some embodiments, the nucleic acid sequence can encode an amino acid sequence comprising at least 80% sequence identity to any one pair of SEQ ID NOs: 1 and 5, 9 and 13, 17 and 21, or 25 and 29. In some embodiments, the nucleic acid sequence can encode an amino acid sequence comprising at least 90% sequence identity to any one pair of SEQ ID NOs: 1 and 5, 9 and 13, 17 and 21, or 25 and 29. In some embodiments, the nucleic acid sequence can encode an amino acid sequence comprising at least 95% sequence identity to any one pair of SEQ ID NOs: 1 and 5, 9 and 13, 17 and 21, or 25 and 29.

In some embodiments, the nucleic acid sequence can encode an amino acid sequence comprising at least 70% sequence identity to SEQ ID NOs: 1 or 5. In some embodiments, the nucleic acid sequence can encode an amino acid sequence comprising at least 70% sequence identity to SEQ ID NOs: 1 and 5. In some embodiments, the nucleic acid sequence can encode an amino acid sequence comprising at least 80% sequence identity to SEQ ID NOs: 1 or 5. In some embodiments, the nucleic acid sequence can encode an amino acid sequence comprising at least 80% sequence identity to SEQ ID NOs: 1 and 5. In some embodiments, the nucleic acid sequence can encode an amino acid sequence comprising at least 90% sequence identity to SEQ ID NOs: 1 or 5. In some embodiments, the nucleic acid sequence can encode an amino acid sequence comprising at least 90% sequence identity to SEQ ID NOs: 1 and 5. In some embodiments, the nucleic acid sequence can encode an amino acid sequence comprising at least 95% sequence identity to SEQ ID NOs: 1 or 5. In some embodiments, the nucleic acid sequence can encode an amino acid sequence comprising at least 95% sequence identity to SEQ ID NOs: 1 and 5. In some embodiments, the nucleic acid sequence can encode an amino acid sequence comprising at least 97% sequence identity to SEQ ID NOs: 1 or 5. In some embodiments, the nucleic acid sequence can encode an amino acid sequence comprising at least 97% sequence identity to SEQ ID NOs: 1 and 5. In some embodiments, the nucleic acid sequence can encode an amino acid sequence comprising at least 99% sequence identity to SEQ ID NOs: 1 or 5. In some embodiments, the nucleic acid sequence can encode an amino acid sequence comprising at least 99% sequence identity to SEQ ID NOs: 1 and 5. In some embodiments, the nucleic acid sequence can encode an amino acid sequence comprising SEQ ID NOs: 1 or 5. In some embodiments, the nucleic acid sequence can encode an amino acid sequence comprising SEQ ID NOs: 1 and 5.

In some embodiments, the nucleic acid sequence can encode an amino acid sequence comprising at least 70% sequence identity to SEQ ID NOs: 9 or 13. In some embodiments, the nucleic acid sequence can encode an amino acid sequence comprising at least 70% sequence identity to SEQ ID NOs: 9 and 13. In some embodiments, the nucleic acid sequence can encode an amino acid sequence comprising at least 80% sequence identity to SEQ ID NOs: 9 or 13. In some embodiments, the nucleic acid sequence can encode an amino acid sequence comprising at least 80% sequence identity to SEQ ID NOs: 9 and 13. In some embodiments, the nucleic acid sequence can encode an amino acid sequence comprising at least 90% sequence identity to SEQ ID NOs: 9 or 13. In some embodiments, the nucleic acid sequence can encode an amino acid sequence comprising at least 90% sequence identity to SEQ ID NOs: 9 and 13. In some embodiments, the nucleic acid sequence can encode an amino acid sequence comprising at least 95% sequence identity to SEQ ID NOs: 9 or 13. In some embodiments, the nucleic acid sequence can encode an amino acid sequence comprising at least 95% sequence identity to SEQ ID NOs: 9 and 13. In some embodiments, the nucleic acid sequence can encode an amino acid sequence comprising at least 97% sequence identity to SEQ ID NOs: 9 or 13. In some embodiments, the nucleic acid sequence can encode an amino acid sequence comprising at least 97% sequence identity to SEQ ID NOs: 9 and 13. In some embodiments, the nucleic acid sequence can encode an amino acid sequence comprising at least 99% sequence identity to SEQ ID NOs: 9 or 13. In some embodiments, the nucleic acid sequence can encode an amino acid sequence comprising at least 99% sequence identity to SEQ ID NOs: 9 and 13. In some embodiments, the nucleic acid sequence can encode an amino acid sequence comprising SEQ ID NOs: 9 or 13. In some embodiments, the nucleic acid sequence can encode an amino acid sequence comprising SEQ ID NOs: 9 and 13.

In some embodiments, the nucleic acid sequence can encode an amino acid sequence comprising at least 70% sequence identity to SEQ ID NOs: 17 or 21. In some embodiments, the nucleic acid sequence can encode an amino acid sequence comprising at least 70% sequence identity to SEQ ID NOs: 17 and 21. In some embodiments, the nucleic acid sequence can encode an amino acid sequence comprising at least 80% sequence identity to SEQ ID NOs: 17 or 21. In some embodiments, the nucleic acid sequence can encode an amino acid sequence comprising at least 80% sequence identity to SEQ ID NOs: 17 and 21. In some embodiments, the nucleic acid sequence can encode an amino acid sequence comprising at least 90% sequence identity to SEQ ID NOs: 17 or 21. In some embodiments, the nucleic acid sequence can encode an amino acid sequence comprising at least 90% sequence identity to SEQ ID NOs: 17 and 21. In some embodiments, the nucleic acid sequence can encode an amino acid sequence comprising at least 9521% sequence identity to SEQ ID NOs: 17 or 21. In some embodiments, the nucleic acid sequence can encode an amino acid sequence comprising at least 9521% sequence identity to SEQ ID NOs: 17 and 21. In some embodiments, the nucleic acid sequence can encode an amino acid sequence comprising at least 97% sequence identity to SEQ ID NOs: 17 or 21. In some embodiments, the nucleic acid sequence can encode an amino acid sequence comprising at least 97% sequence identity to SEQ ID NOs: 17 and 21. In some embodiments, the nucleic acid sequence can encode an amino acid sequence comprising at least 99% sequence identity to SEQ ID NOs: 17 or 21. In some embodiments, the nucleic acid sequence can encode an amino acid sequence comprising at least 99% sequence identity to SEQ ID NOs: 17 and 21. In some embodiments, the nucleic acid sequence can encode an amino acid sequence comprising SEQ ID NOs: 17 or 21. In some embodiments, the nucleic acid sequence can encode an amino acid sequence comprising SEQ ID NOs: 17 and 21.

In some embodiments, the nucleic acid sequence can encode an amino acid sequence comprising at least 70% sequence identity to SEQ ID NOs: 25 or 29. In some embodiments, the nucleic acid sequence can encode an amino acid sequence comprising at least 70% sequence identity to SEQ ID NOs: 25 and 29. In some embodiments, the nucleic acid sequence can encode an amino acid sequence comprising at least 80% sequence identity to SEQ ID NOs: 25 or 29. In some embodiments, the nucleic acid sequence can encode an amino acid sequence comprising at least 80% sequence identity to SEQ ID NOs: 25 and 29. In some embodiments, the nucleic acid sequence can encode an amino acid sequence comprising at least 90% sequence identity to SEQ ID NOs: 25 or 29. In some embodiments, the nucleic acid sequence can encode an amino acid sequence comprising at least 90% sequence identity to SEQ ID NOs: 25 and 29. In some embodiments, the nucleic acid sequence can encode an amino acid sequence comprising at least 95% sequence identity to SEQ ID NOs: 25 or 29. In some embodiments, the nucleic acid sequence can encode an amino acid sequence comprising at least 95% sequence identity to SEQ ID NOs: 25 and 29. In some embodiments, the nucleic acid sequence can encode an amino acid sequence comprising at least 97% sequence identity to SEQ ID NOs: 25 or 29. In some embodiments, the nucleic acid sequence can encode an amino acid sequence comprising at least 97% sequence identity to SEQ ID NOs: 25 and 29. In some embodiments, the nucleic acid sequence can encode an amino acid sequence comprising at least 99% sequence identity to SEQ ID NOs: 25 or 29. In some embodiments, the nucleic acid sequence can encode an amino acid sequence comprising at least 99% sequence identity to SEQ ID NOs: 25 and 29. In some embodiments, the nucleic acid sequence can encode an amino acid sequence comprising SEQ ID NOs: 25 or 29. In some embodiments, the nucleic acid sequence can encode an amino acid sequence comprising SEQ ID NOs: 25 and 29.

The nucleic acid of this disclosure can be encapsulated or coupled to a vector. In some embodiments, the vector is configured to deliver any one or more of the nucleic acids described herein to a subject. In some cases, the vector is a non-viral vector. In some embodiments, the non-viral vector is a lipid nanoparticle (LNP), a liposome, a cationic lipid, a polymer, an inorganic nanoparticle, an organic nanoparticle, an extracellular vesicle (e.g., a microvesicle or an exosome), or a peptide. In some embodiments, the vector can be a viral vector. In some cases, the viral vector is an adenoviral vector, an associated-adeno viral vector, a lentiviral vector, a retroviral vector, or a herpes simplex viral vector.

In some embodiments, the vector comprises a first nucleic acid sequence encoding a heavy chain variable region comprising an amino acid sequence as set forth in SEQ ID NO: 1, or encoding a heavy chain variable region comprising an H-CDR1, a H-CDR2, and a H-CDR3 as set forth in the amino acid sequence of SEQ ID NO: 1, and/or a second nucleic acid encoding a light chain variable region comprising an amino acid sequence as set forth in SEQ ID NO: 5, or encoding a light chain variable region comprising an L-CDR1, an L-CDR2, and an L-CDR3 as set forth in the amino acid sequence of SEQ ID NO: 5.

In some embodiments, the vector comprises a first nucleic acid sequence encoding a heavy chain variable region comprising an amino acid sequence as set forth in SEQ ID NO: 9, or encoding a heavy chain variable region comprising an H-CDR1, a H-CDR2, and a H-CDR3 as set forth in the amino acid sequence of SEQ ID NO: 9, and/or a second nucleic acid encoding a light chain variable region comprising an amino acid sequence as set forth in SEQ ID NO: 13, or encoding a light chain variable region comprising an L-CDR1, an L-CDR2, and an L-CDR3 as set forth in the amino acid sequence of SEQ ID NO: 13.

In some embodiments, a first nucleic acid sequence encoding a heavy chain variable region comprising an amino acid sequence as set forth in SEQ ID NO: 17, or encoding a heavy chain variable region comprising an H-CDR1, a H-CDR2, and a H-CDR3 as set forth in the amino acid sequence of SEQ ID NO: 17, and/or a second nucleic acid encoding a light chain variable region comprising an amino acid sequence as set forth in SEQ ID NO: 21, or encoding a light chain variable region comprising an L-CDR1, an L-CDR2, and an L-CDR3 as set forth in the amino acid sequence of SEQ ID NO: 21.

In some embodiments, the vector comprises a first nucleic acid sequence encoding a heavy chain variable region comprising an amino acid sequence as set forth in SEQ ID NO: 25, or encoding a heavy chain variable region comprising an H-CDR1, a H-CDR2, and a H-CDR3 as set forth in the amino acid sequence of SEQ ID NO: 25, and/or a second nucleic acid encoding a light chain variable region comprising an amino acid sequence as set forth in SEQ ID NO: 29, or encoding a light chain variable region comprising an L-CDR1, an L-CDR2, and an L-CDR3 as set forth in the amino acid sequence of SEQ ID NO: 29.

### Methods

The anti-human κ light chain antibodies and/or human κ light chain binding antibody fragments described herein can be used to identify, detect, and treat misfolded κ light chain (e.g., K light chain aggregates or amyloid deposits), and diseases or disorders associated therewith. This disclosure also provides and describes methods for promoting clearance of misfolded κ light chains with the anti-human κ light chain antibodies and/or human κ light chain binding antibody fragments described herein. In another aspect, a method of making or manufacturing the anti-human κ light chain antibodies and/or human κ light chain binding antibody fragments is also provided and described herein.

### Methods of Detecting κ Light Chains

Provided and described herein are methods of diagnosing amyloidosis in a sample, the method comprising contacting a biological sample obtained from a subject with the anti-human κ light chain antibodies or human κ light chain binding antibody fragments described herein. Also provided herein are methods of detecting free light chain polypeptides in a sample, the method comprising contacting the biological sample with the anti-human κ light chain antibodies or human κ light chain binding antibody fragments described herein. Further provided are methods of detecting amyloid deposits in a sample, the method comprising contacting the biological sample with the anti-human κ light chain antibodies or human κ light chain binding antibody fragments described herein. In some embodiments, the sample can be from blood, serum, plasma, solid tissue, and any combination thereof. In some embodiments, the sample is obtained from a subject.

As described, certain embodiments of the present disclosure are directed to methods of measuring light chain immunoglobulins. The anti-human κ light chain antibodies or human κ light chain binding antibody fragments disclosed herein can be used to measure any immunoglobulin light chain or aggregate thereof. In some embodiments, the anti-human κ light chain antibodies or human κ light chain binding antibody fragments can be used to measure an immunoglobulin light chain or an aggregate thereof in vivo. In some embodiments, the anti-human κ light chain antibodies or human κ light chain binding antibody fragments can be used to measure an immunoglobulin light chain or an aggregate thereof in vitro or ex vivo. In some embodiments, the anti-human κ light chain antibodies or human κ light chain binding antibody fragments can be used to measure an immunoglobulin light chain or an aggregate thereof in a biological sample collected from a subject. In some embodiments, the biological sample can be a plasma sample, a serum sample, a blood sample, a tissue sample (e.g., a tissue biopsy), a solid tissue sample, a liquid biopsy, a urine sample, a bone marrow sample, a cerebrospinal fluid sample, or any combination thereof. In some embodiments, the biological sample can be a blood sample. In some embodiments, the biological sample can be a serum sample. In some embodiments, the biological sample can be a bone marrow sample. In some embodiments, the biological sample comprises an immune cell (e.g., lymphocyte, macrophage, monocyte, or dendritic cell). In some embodiments, the sample can be a tissue sample, including but not limited to a cardiac tissue, a kidney tissue. a pulmonary tissue (e.g., a lung), an intestinal tissue (e.g., a colon), a gastrointestinal (GI) tissue (e.g., a stomach, an esophagus), a skin tissue, a subcutis tissue, a soft tissue, a tendon tissue, a bone tissue, a synovium, a gall bladder tissue, a pharyngeal tissue (e.g., a nasopharynx), a spleen tissue, a lymph node, a neural tissue (e.g., a brain, a spinal cord), or an immune tissue (e.g., bone marrow, thymus, spleen, lymph nodes, tonsils, adenoids). In some embodiments, the tissue sample can be a cardiac tissue. In some embodiments, the tissue sample can be a renal tissue (e.g., kidney tissue). In some embodiments, the tissue sample can be a gastrointestinal tissue or a colon tissue. In some embodiments, the method further comprises quantifying a level of an immunoglobulin light chain or an aggregate thereof in a biological sample collected from a subject. In some embodiments, the quantifying comprises measuring a level of an immunoglobulin light chain or an aggregate thereof in a biological sample collected from a subject. In some embodiments, the measuring comprises detecting a level of an immunoglobulin in a biological sample obtained from a subject by ELISA, western blot, mass spectrometry, biolayer interferometry, immunohistochemistry, histology, or microscopy. In some embodiments, the quantifying comprises analyzing a level of an immunoglobulin light chain or an aggregate thereof in a biological sample collected from a subject. In some embodiments, the immunoglobulin light chain or the aggregate thereof comprises a κ light chain or an aggregate thereof. In some embodiments, the immunoglobulin light chain or the aggregate thereof comprises a κ light chain amyloid. In some embodiments, the immunoglobulin light chain or the aggregate thereof comprises an alkylated κ light chain. In some embodiments, the immunoglobulin light chain or the aggregate thereof comprises a misfolded κ light chain.

In some embodiments, measuring light chain immunoglobulins can comprise measuring misfolded κ light chains, κ light chain amyloid deposits, κ light chain aggregates, or alkylated κ light chains. In some embodiments, measuring light chain immunoglobulins can comprise measuring in a sample obtained from a subject at least a 10%, 20%, 30%, 40%, 50%, 60% 70%, 80%, or 90% increase in a level of misfolded κ light chains in the sample obtained from the subject compared to a control sample. In some embodiments, measuring light chain immunoglobulins can comprise measuring in a sample obtained from a subject at least a 10%, 20%, 30%, 40%, 50%, 60% 70%, 80%, or 90% increase in a level of κ light chain aggregates in the sample obtained from the subject compared to a control sample. In some embodiments, measuring light chain immunoglobulins can comprise measuring in a sample obtained from a subject at least a 10%, 20%, 30%, 40%, 50%, 60% 70%, 80%, or 90% increase in a level of alkylated κ light chains in the sample obtained from the subject compared to a control sample. In some embodiments, measuring light chain immunoglobulins can comprise measuring in a sample obtained from a subject at least a 10%, 20%, 30%, 40%, 50%, 60% 70%, 80%, or 90% increase in a level of κ light chain amyloids in the sample obtained from the subject compared to a control sample. In some embodiments, the control sample can be a biological sample obtained from a control subject. In some embodiments, the control sample can be obtained from a healthy individual, a subject that does not have an amyloidosis (e.g., antibody light chain amyloidosis), or a subject that does not have a plasma cell disorder (e.g., plasma cell dyscrasias). In some embodiments, measuring light chain immunoglobulins can comprise performing an ELISA assay, a western blot, biolayer interferometry, or any other method of measuring protein levels in a biological sample.

In some embodiments, the method can comprise measuring light chain fibrils. As such, the antibodies described herein can be used to identify a subject that has a disease or condition characterized by the formation of immunoglobulin light chain deposits, e.g., amyloid fibril deposits. In some embodiments, the anti-human κ light chain antibodies or human κ light chain binding antibody fragments can be used to identify (e.g., diagnose) a subject as having an amyloidosis. In some embodiments, the anti-human κ light chain antibodies or human κ light chain binding antibody fragments can be used to identify a subject as having a systemic amyloidosis. In some embodiments, the anti-human κ light chain antibodies or human κ light chain binding antibody fragments can be used to identify a subject as having an AL amyloidosis. In some embodiments, the anti-human κ light chain antibodies or human κ light chain binding antibody fragments can be used to identify a subject as having a plasma cell dyscrasia. In some embodiments, the anti-human κ light chain antibodies or human κ light chain binding antibody fragments can be used to identify a subject as having a monoclonal gammopathy of unknown significance (MGUS). In some embodiments, the anti-human κ light chain antibodies or human κ light chain binding antibody fragments can be used to identify a subject as having multiple myeloma (MM). In some embodiments, the anti-human κ light chain antibodies or human κ light chain binding antibody fragments can be used to identify a subject as having plasma cell leukemia (PCL). In some embodiments, the anti-human κ light chain antibodies or human κ light chain binding antibody fragments can be used to identify a subject as having light chain deposition disease (LCDD).

In some embodiments, the anti-human κ light chain antibody or human κ light chain binding antibody fragment can comprise one or more labels, covalently or non-covalently bound to the antibody or antigen binding fragment. Any label known in the art can be used to increase the ability of anti-human κ light chain antibody or human κ light chain binding antibody fragment to be detected and/or visualized. In some embodiments, the label can comprise a chemical label. In some embodiments, the label can comprise an enzymatic label. In some embodiments, label can comprise a substrate, which is detectable in the presence of a particular enzyme. In some embodiments, the label can be a fluorescent label. In some embodiments, the label can be a radioisotope label.

Certain embodiments of the present disclosure are directed to methods of measuring light chain immunoglobulins in vivo using an anti-human κ light chain antibody or human κ light chain binding antibody fragment disclosed herein. In some embodiments, the anti-human κ light chain antibody or human κ light chain binding antibody fragment comprises a detectable probe. In some embodiments, the anti-human κ light chain antibody or human κ light chain binding antibody fragment can be a contrast-conjugated antibody. In some embodiments, the method can be a diagnostic medical imaging method, e.g., a PET/CT, SPECT, MRI, or any combination thereof.

In some embodiments, the measuring the light chain fibrils can comprise measuring misfolded κ light chains, κ light chain amyloid deposits, κ light chain aggregates, or alkylated κ light chains. In some embodiments, measuring light chain fibrils can comprise measuring in a sample obtained from a subject at least a 10%, 20%, 30%, 40%, 50%, 60% 70%, 80%, or 90% increase in a level of misfolded κ light chains in the sample obtained from the subject compared to a control sample. In some embodiments, measuring light chain fibrils can comprise measuring in a sample obtained from a subject at least a 10%, 20%, 30%, 40%, 50%, 60% 70%, 80%, or 90% increase in a level of κ light chain aggregates in the sample obtained from the subject compared to a control sample. In some embodiments, measuring light chain fibrils can comprise measuring in a sample obtained from a subject at least a 10%, 20%, 30%, 40%, 50%, 60% 70%, 80%, or 90% increase in a level of alkylated κ light chains in the sample obtained from the subject compared to a control sample. In some embodiments, measuring light chain fibrils can comprise measuring in a sample obtained from a subject at least a 10%, 20%, 30%, 40%, 50%, 60% 70%, 80%, or 90% increase in a level of κ light chain amyloids in the sample obtained from the subject compared to a control sample. In some embodiments, the control sample can be a biological sample obtained from a control subject. In some embodiments, the control sample can be obtained from a healthy individual, a subject that does not have an amyloidosis (e.g., antibody light chain amyloidosis), or a subject that does not have a plasma cell disorder (e.g., plasma cell dyscrasias). In some embodiments, measuring light chain fibrils can comprise performing an ELISA assay, a western blot, biolayer interferometry, or any other method of measuring protein levels in a biological sample.

In some embodiments, the subject can be a mammal. In some embodiments, the subject can be a primate. In some embodiments, the subject can be a human. In some embodiments, the subject can be a non-human primate. In some embodiments, the subject can be a monkey, a chimpanzee, a macaque, a cat, a dog, a sheep, a goat, a chicken, a camel, or a llama.

### Methods of Treating and or Reducing Misfolded κ Light Chains

Provided and described herein are methods of promoting the clearance of a κ light chain aggregate by monocytic cells or macrophages, the method comprising contacting the misfolded κ light chain or κ light chain aggregate with the anti-human κ light chain antibodies or human κ light chain binding antibody fragments described herein, thereby promoting the clearance of the misfolded κ light chain or κ light chain aggregate by a phagotyic cell (e.g., monocytic cells or macrophages). In some embodiments, the contacting occurs in vivo. In some embodiments, the contacting occurs in vitro or ex vivo.

Clearance of a κ light chain aggregate by the anti-human κ light chain antibodies or human κ light chain binding antibody fragments of this disclosure can be measured. In some embodiments, the κ light chain clearance can be measured upon detecting in a subject, in a tissue obtained from a subject, and/or in vitro a level of the κ light chain aggregate using any method known in the art. Common methods for detecting the κ light chain aggregate include, but are not limited to, in vivo protein tagging methods, immunological assays (e.g., ELISA), immunostaining, immunohistology (e.g., immunofluorescent reporters), reporters, western blotting, and the like.

Clearance of the κ light chain aggregate can be determined upon measuring a decrease in the κ light chain aggregate. In some cases, the anti-κ light chain antibodies or κ light chain binding fragments thereof described herein can decrease a level of a κ light chain aggregate in a subject, a sample, or a cell by at least about 10%, 20%, 30%, 40%, 50%, 60%, 70%, 80%, 90%, or more compared to a level of the misfolded κ light chain or κ light chain aggregate in a similar subject, sample, or cell that has not been contacted with anti-κ light chain antibodies or κ light chain binding fragments thereof. In some cases, the anti-κ light chain antibodies or κ light chain binding fragments thereof described herein can decrease a level of a misfolded κ light chain or κ light chain aggregate in a subject, a sample, or a cell by at least about 10%, 20%, 30%, 40%, 50%, 60%, 70%, 80%, 90%, or more compared to a level of the misfolded κ light chain or κ light chain aggregate in a similar subject, sample, or cell that has been contacted with a variable region-targeting anti-K light chain antibody.

For example, the level of the misfolded κ light chain or κ light chain aggregate can be measured to decrease at a given one or more time points compared to the level of the misfolded κ light chain or κ light chain aggregate in a subject or sample acquired at an earlier time point (e.g., a reference or baseline timepoint).

In some cases, a decrease of the κ light chain aggregate by the anti-K light chain antibodies or κ light chain binding fragments thereof can be a kinetic measurement, such as an increase in a decay rate of the misfolded κ light chain or κ light chain aggregate level.

In some cases, the anti-K light chain antibodies or κ light chain binding fragments thereof described herein can increase a decay rate of a misfolded κ light chain or κ light chain aggregate in a subject, a sample, or a cell by at least about 10%, 20%, 30%, 40%, 50%, 60%, 70%, 80%, 90%, or more compared to a level of the κ light chain aggregate in a similar subject, sample, or cell that has not been contacted with anti-K light chain antibodies or κ light chain binding fragments thereof. In some cases, the anti-K light chain antibodies or κ light chain binding fragments thereof described herein can increase a decay rate of a κ light chain aggregate in a subject, a sample, or a cell by no more than about 10%, 20%, 30%, 40%, 50%, 60%, 70%, 80%, 90%, or less compared to a level of the misfolded κ light chain or κ light chain aggregate in a similar subject, sample, or cell that has not been contacted with anti-K light chain antibodies or κ light chain binding fragments thereof.

The decay rate of the level of the κ light chain aggregate can be measured by determining or calculating a half-life or a decay rate constant of the κ light chain aggregate in a subject, a sample, or a cell. The half-life and decay rate constants of the κ light chain aggregate can be measured or extrapolated from fitted data curves consisting of a series of timepoints according to the following equations or by any other means know in the art for determining such parameters of the κ light chain aggregate:

$$N ( t ) = N 0 {e}^{- λ t} \qquad Equation I$$

$$\frac{t 1}{2} = \frac{ln ( 2 )}{λ} \qquad Equation II$$

In some embodiments, the kinetic measurement can include a decrease in a half-life of the misfolded κ light chain or κ light chain aggregate measured in a subject, sample, or a cell over time.

In some cases, the anti-K light chain antibodies or κ light chain binding fragments thereof described herein can decrease a half-life of a misfolded κ light chain or κ light chain aggregate in a subject, a sample, or a cell by at least about 10%, 20%, 30%, 40%, 50%, 60%, 70%, 80%, 90%, or more compared to a level of the κ light chain aggregate in a similar subject, sample, or cell that has not been contacted with anti-K light chain antibodies or κ light chain binding fragments thereof. In some cases, the anti-K light chain antibodies or κ light chain binding fragments thereof described herein can decrease a half-life of a misfolded κ light chain or κ light chain aggregate in a subject, a sample, or a cell by no more than about 10%, 20%, 30%, 40%, 50%, 60%, 70%, 80%, 90%, or less compared to a level of the misfolded κ light chain or κ light chain aggregate in a similar subject, sample, or cell that has not been contacted with anti-K light chain antibodies or κ light chain binding fragments thereof.

In some cases, the anti-K light chain antibodies or κ light chain binding fragments thereof described herein can decrease a half-life of a misfolded κ light chain or κ light chain aggregate in a subject, a sample, or a cell by no more than about 10%, 20%, 30%, 40%, 50%, 60%, 70%, 80%, 90%, or less compared to a level of the misfolded κ light chain or κ light chain aggregate in a similar subject, sample, or cell that has been contacted with a variable region-targeting anti-K light chain antibodies or κ light chain binding fragments thereof.

In some cases, the half-life of the κ light chain aggregate after exposure of a subject, sample, or cell to one of the anti-K light chain antibodies or κ light chain binding fragments thereof of this disclosure can be no more than about 1 day, 2 days, 3 days, 4 days, 5 days, 6 days, 7 days, about 2 weeks, 3 weeks, 4 weeks, 5 weeks, 6 weeks, 7 weeks, 8 weeks, 10 weeks, 12 weeks, 4 months, 5 months, 6 months, 12 months, 18 months, 24 months, or less.

In some cases, the anti-K light chain antibodies or κ light chain binding fragments thereof described herein can increase a decay rate constant of a κ light chain aggregate in a subject, a sample, or a cell by at least about 10%, 20%, 30%, 40%, 50%, 60%, 70%, 80%, 90%, or more compared to a level of the misfolded κ light chain or κ light chain aggregate in a similar subject, sample, or cell that has not been contacted with anti-K light chain antibodies or κ light chain binding fragments thereof. In some cases, the anti-κ light chain antibodies or κ light chain binding fragments thereof described herein can increase a decay rate constant of a misfolded κ light chain or κ light chain aggregate in a subject, a sample, or a cell by no more than about 10%, 20%, 30%, 40%, 50%, 60%, 70%, 80%, 90%, or less compared to a level of the misfolded κ light chain or κ light chain aggregate in a similar subject, sample, or cell that has not been contacted with anti-κ light chain antibodies or κ light chain binding fragments thereof.

Clearance of a misfolded κ light chain or κ light chain aggregate by the anti-human κ light chain antibodies or human κ light chain binding antibody fragments of this disclosure can be measured over a time period. The clearance of the misfolded κ light chain or κ light chain aggregate can be measured over a course of at least at least about 1 hours, 2 hours, 3 hours, 4 hours, 5 hours, 10 hours, 12 hours, 24 hours, 48 hours, 3 days, 4 day, 5 days, 6 days, 7 days, 8 days, 9 days, 10, day, 2 weeks, 3 weeks, 4 weeks, 3 months, 6 months, 1 year, or longer.

In some embodiments, the anti-human κ light chain antibodies or human κ light chain binding antibody fragments provided herein can reduce a level of a misfolded κ light chain or κ light chain aggregate from a tissue of a subject in no more than 6 months, 5 months, 4 months, 3 months, 2 months, 1 month, 3 weeks, 2 weeks, 1 week, 6 days, 5 days, 4 days, 3 days, or less.

In some embodiments, the anti-human κ light chain antibodies or human κ light chain binding antibody fragments disclosed herein can reduce a level of a misfolded κ light chain or κ light chain aggregate in a tissue of a subject by 50% in no more than 3 months, 2 months, 1 month, 3 weeks, 2 weeks, 1 week, 6 days, 5 days, 4 days, 3 days, or less.

In some embodiments, the method further comprises recruiting a monocytic cell or a macrophage to a misfolded κ light chain (e.g., κ light chain aggregate, alkylated κ light chain, or κ light chain amyloid) in the subject. In some embodiments, the monocytic cell can be a macrophage or a dendritic cell. In some embodiments, the macrophage expresses F4/80. In some embodiments, recruiting a monocytic cell or a macrophage to a misfolded κ light chain can comprise increasing a number of monocytes or a number of macrophages in contact with a misfolded κ light chain or a κ light chain amyloidoma compared to a control. In some embodiments, recruiting a monocytic cell can comprise increasing a number of monocytes in contact with a misfolded κ light chain or a κ light chain amyloidoma by at least about 10%, 20%, 30%, 40%, 50%, 60%, 70%, 80%, 90%, 100%, 150%, 20%, 250%, 300%, 350%, 400%, or more compared to a control. In some embodiments, recruiting a macrophage can comprise increasing a number of macrophages in contact with a misfolded κ light chain or a κ light chain amyloidoma by at least about 10%, 20%, 30%, 40%, 50%, 60%, 70%, 80%, 90%, 100%, 150%, 20%, 250%, 300%, 350%, 400%, or more compared to a control. In some embodiments, the recruiting a macrophage can comprise increasing a number of macrophages in contact with a misfolded κ light chain or a κ light chain amyloidoma compared to a control for a period of at least about 1 day, 2 days, 3 days, 4 days, 5 days, 6 days, 7 days, 8 days, 9 days, 10 days, 14 days, 28 days, 2 months, 3 months, 4 months, 5 months, 6 months, 9 months, 12 months, 18 months, 2 years, or more after contacting the κ light chain aggregate with the anti-human κ light chain antibodies or binding antibody fragments thereof. In some embodiments, the recruiting a macrophage can comprise increasing a number of macrophages in contact with a misfolded κ light chain or amyloidoma by at least about 100% compared to a control for a period of at least about 5 days after contacting the κ light chain aggregate with the anti-human κ light chain antibodies or binding antibody fragments thereof. In some embodiments, the recruiting a macrophage can comprise increasing a number of macrophages in contact with a misfolded κ light chain or amyloidoma by about 200% compared to a control for a period of about 6 days after contacting the κ light chain aggregate with the anti-human κ light chain antibodies or binding antibody fragments thereof. In some embodiments, the recruiting a macrophage can comprise increasing a number of macrophages in contact with a misfolded κ light chain or amyloidoma by about 300% compared to a control for a period of about 6 days after contacting the κ light chain aggregate with the anti-human κ light chain antibodies or binding antibody fragments thereof. In some embodiments, the control can be a number of monocytes or macrophages contacting misfolded κ light chains or amyloidomas prior to contacting with the anti-human κ light chain antibodies or binding antibody fragments thereof (e.g., a within-subject or a within-sample comparison). In some embodiments, the control can be a number of a number of monocytes or macrophages contacting misfolded κ light chains or amyloidomas in an absence of anti-human κ light chain antibodies or binding antibody fragments thereof (e.g., an untreated sample or subject).

Also provided and described are methods of increasing clearance of a κ light chain aggregates, the method comprising contacting the κ light chain aggregate with the anti-human κ light chain antibodies or human κ light chain binding antibody fragments described herein, thereby increasing the clearance of a κ light chain aggregates.

In some embodiments, increasing clearance of κ light chain aggregates comprises decreasing a level of κ light chain aggregates in a biological sample compared to a control. In some embodiments, the increasing clearance of κ light chain aggregates can comprise decreasing a level of κ light chain aggregates in a biological sample at a faster rate than a control. In some embodiments, the increasing clearance of κ light chain aggregates can comprise decreasing a level of κ light chain aggregates in a biological sample by at least about 1.5-fold, 2-fold, 3-fold, 4-fold, 5-fold, 6-fold, 7-fold, 8-fold, 9-fold, 10-fold, or more compared to a control after a time period following contacting with the anti-human κ light chain antibodies or binding antibody fragments thereof. In some embodiments, the increasing clearance of κ light chain aggregates can comprise decreasing a level of κ light chain aggregates in a biological sample by at least about 1.5-fold compared to a control after 12 hours after contacting with the anti-human κ light chain antibodies or binding antibody fragments thereof. In some embodiments, the increasing clearance of κ light chain aggregates can comprise decreasing a level of κ light chain aggregates in a biological sample by at least about 2-fold compared to a control after 24 hours after contacting with the anti-human κ light chain antibodies or binding antibody fragments thereof. In some embodiments, the biological is a sample obtained from a subject or derived from the subject. In some embodiments, the biological sample is a serum sample. In some embodiments, the biological sample is a serum sample.

Further provided and described are methods of reducing κ light chain aggregates in a subject, the method comprising administering the anti-human κ light chain antibodies or human κ light chain binding antibody fragments described herein to the subject, thereby reducing the κ light chain aggregates in the subject.

In some embodiments, reducing κ light chain aggregates in a subject can comprise reducing a number of κ light chain aggregates (e.g., amyloidomas) in the subject by at least about 10%, 20%, 30%, 40%, 50%, 60%, 70%, 80%, 90% or more compared to a control subject after at least 1 day, 2 days, 3 days, 4 days, 5 days, 7 days, 10 days, 12 days, 15 days, or longer after administration of the anti-human κ light chain antibodies or human κ light chain binding antibody fragments thereof.

In some embodiments, reducing κ light chain aggregates in a subject can comprise reducing a size of κ light chain aggregates (e.g., amyloidomas) in the subject by at least about 10%, 20%, 30%, 40%, 50%, 60%, 70%, 80%, 90%, or more compared to a control subject for at least 24 hours after administration of the anti-human κ light chain antibodies or binding antibody fragments thereof.

In some embodiments, reducing κ light chain aggregates in a subject can comprise increasing phagocytic uptake of κ light chain aggregates in the subject. In some embodiments, increasing phagocytic uptake of κ light chain aggregates (e.g., κ light chain amyloid, κ light chain fibrils) can comprise increasing a level of κ light chain amyloid in phagocytes of the subject by at least about 1-fold, 2-fold, 3-fold, 4-fold, or more compared to a control subject. In some embodiments, increasing phagocytic uptake of κ light chain aggregates can comprise increasing a level of κ light chain amyloid in phagocytes of the subject by about 2-fold compared to a control subject for a period of from about 1 day to about 6 days after administration of the anti-human κ light chain antibodies or binding antibody fragments thereof. In some embodiments, increasing phagocytic uptake of κ light chain aggregates can comprise increasing a level of κ light chain amyloid in phagocytes of the subject by about 1-fold compared to a control subject for a period of from about 1 day to about 8 days after administration of the anti-human κ light chain antibodies or binding antibody fragments thereof.

In some embodiments, reducing κ light chain aggregates in a subject can comprise reducing a size of κ light chain aggregates (e.g., amyloidomas) in the subject by at least about 10% compared to a control subject for a period of at least 24 hours, 48 hours, 72, hours, 96 hours, 5 days, 6 days, 7 days, 8 days, 9 days, 10 days, 12 days, 14 days, 21 days, 1 month, 2 months, 3 months, or longer after administration of the anti-human κ light chain antibodies or binding antibody fragments thereof. In some embodiments, reducing κ light chain aggregates in a subject can comprise reducing an area of κ light chain aggregates (e.g., amyloidomas) in the subject by at least about 10%, 20%, 30%, 40%, 50%, 60%, 70%, 80%, or 90% compared to a control subject. In some embodiments, an area of a κ light chain aggregate (e.g., amyloidomas) in the subject is reduced by at least about 30% after about 48 hours following administration of the anti-human κ light chain antibodies or binding antibody fragments thereof. In some embodiments, an area of a κ light chain aggregate (e.g., amyloidomas) in the subject is reduced by at least about 50% after about 96 hours following administration of the anti-human κ light chain antibodies or binding antibody fragments thereof.

In some embodiments, the method can result in a decrease in the level, the size, or the number of the kappa light chain aggregate in a cardiac tissue, a renal tissue, a pulmonary tissue, an intestinal tissue, a gastrointestinal (GI) tissue, a soft tissue, a synovial tissue, a spleen tissue, or an immune tissue compared to a level of the kappa light chain aggregate in a similar tissue prior to the contacting.

In some embodiments, the method can result in a decrease in the level of the kappa light chain aggregate in a cardiac tissue compared to a level of the kappa light chain aggregate in a similar cardiac tissue prior to the contacting. In some embodiments, the method can result in a decrease in the level of the kappa light chain aggregate in a similar renal tissue compared to a level of the kappa light chain aggregate in a similar renal tissue prior to the contacting. In some embodiments, the method can result in a decrease in the level of the kappa light chain aggregate in a colon tissue compared to a level of the kappa light chain aggregate in a colon tissue prior to the contacting.

In some embodiments, the method can result in a decrease in the level or the number of the kappa light chain aggregate in a cardiac tissue, a renal tissue, a pulmonary tissue, an intestinal tissue, a gastrointestinal (GI) tissue, a soft tissue, a synovial tissue, a spleen tissue, or an immune tissue compared to a level or a number or a number of the kappa light chain aggregate in a similar tissue prior to the administering

In some embodiments, the method can result in a decrease in the level or the number of the kappa light chain aggregate in a cardiac tissue compared to a level or a number of the kappa light chain aggregate in a similar cardiac tissue prior to the administering. In some embodiments, the method can result in a decrease in the level or the number of the kappa light chain aggregate in a renal tissue compared to a level or a number of the kappa light chain aggregate in a similar renal tissue prior to the administering. In some embodiments, the method can result in a decrease in the level or the number of the kappa light chain aggregate in a similar colon tissue compared to a level or a number of the kappa light chain aggregate in a similar colon tissue prior to the administering.

Provided and described herein are methods of treating a disease or condition characterized by κ light chain aggregates, the method comprising administering the anti-human κ light chain antibodies or human κ light chain binding antibody fragments described herein to the subject, thereby reducing the number of κ light chain aggregates in the subject. Provided and described are methods of treating AL amyloidosis (e.g., in a subject having κ light chain aggregates), the method comprising administering the anti-human κ light chain antibodies or human κ light chain binding antibody fragments described herein to the subject, thereby reducing the number of κ light chain aggregates in the subject.

The instant disclosure also provides an anti-human kappa light chain antibody or human kappa light chain binding fragment thereof for use in the manufacture of a medicament for the treatment of a disease or condition characterized by kappa light chain aggregates in the subject.

In some embodiments, the subject has a plasma cell disorder. In some embodiments, the plasma cell disorder comprises plasma cell dyscrasia. In some embodiments, the plasma cell dyscrasia is selected from a monoclonal gammopathy of unknown significance (MGUS), multiple myeloma (MM), plasma cell leukemia (PCL), and any combination thereof. In some cases, the subject has a disease or condition characterized by kappa light chain aggregates. In some cases, the disease or condition characterized by kappa light chain aggregates is a plasma cell disorder, systemic amyloidosis, or light chain amyloidosis. In some cases, the disease or condition characterized by kappa light chain aggregates is an amyloidosis. In some embodiments, the disease or condition characterized by kappa light chain aggregates is a systemic amyloidosis. In certain embodiments, the disease or condition characterized by kappa light chain aggregates is a light chain amyloidosis. In certain embodiments, the disease or condition characterized by kappa light chain aggregates can be a cardiac amyloidosis. In certain embodiments, the disease or condition characterized by kappa light chain aggregates can be a renal amyloidosis. In some embodiments, the disease or condition characterized by kappa light chain aggregates is a plasma cell dyscrasias. In some cases, the plasma cell dyscrasias is amyloidosis, monoclonal gammopathy of unknown significance (MGUS), multiple myeloma (MM), plasma cell leukemia (PCL), or any combination thereof.

Certain uses of the anti-human κ light chain antibodies or human κ light chain binding antibody fragments described herein are directed to methods of treating a disease or condition in a subject in need thereof, comprising administering an anti-human κ light chain antibody or human κ light chain binding antibody fragment described herein. In some embodiments, the disease or condition comprises an amyloidosis. In some embodiments, the disease or condition comprises a systemic amyloidosis. In some embodiments, the disease or condition comprises an AL amyloidosis. In some embodiments, the disease or condition comprises a plasma cell dyscrasia. In some embodiments, the disease or condition comprises a monoclonal gammopathy of unknown significance (MGUS). In some embodiments, the disease or condition comprises multiple myeloma (MM). In some embodiments, the disease or condition comprises plasma cell leukemia (PCL). In some embodiments, the disease or condition comprises light chain deposition disease (LCDD).

Certain uses of the anti-human κ light chain antibodies or human κ light chain binding antibody fragments described herein are directed to methods of reducing and/or clearing an immunoglobulin light chain aggregate, e.g., an amyloid fibril, in a subject in need thereof, comprising administering an anti-human κ light chain antibody or human κ light chain binding antibody fragment described herein. In some embodiments, the subject can have one or more immunoglobulin light chain aggregate, e.g., amyloid fibril, in a tissue of a subject or a sample obtained from a subject.

In some embodiments, the anti-human κ light chain antibody or human κ light chain binding antibody fragment described herein can reduce or clear a misfolded κ light chain or aggregate from a tissue of a subject. In some embodiments, the tissue can be of a cardiac tissue, a kidney tissue. a pulmonary tissue (e.g., a lung), an intestinal tissue (e.g., a colon), a gastrointestinal (GI) tissue (e.g., a stomach, an esophagus), a skin tissue, a subcutis tissue, a soft tissue, a tendon tissue, a bone tissue, a synovium, a gall bladder tissue, a pharyngeal tissue (e.g., a nasopharynx), a spleen tissue, a lymph node, a neural tissue (e.g., a brain, a spinal cord), or an immune tissue (e.g., bone marrow, thymus, spleen, lymph nodes, tonsils, adenoids).

In some embodiments, the subject can have one or more immunoglobulin light chain aggregate in a subregion of a heart, including a ventricle, an atrium, a myocardium, an endocardium, an epicardium, a pericardium, a septum, a valve, or a Trabeculae Carneae. In some embodiments, the subject can have one or more immunoglobulin light chain aggregate, e.g., amyloid fibril, in the heart. In some embodiments, the subject can have one or more immunoglobulin light chain aggregate in a subregion of a heart, including a ventricle, an atrium, a myocardium, an endocardium, an epicardium, a pericardium, a septum, a valve, or a Trabeculae Carneae. In some embodiments, the subject can have one or more immunoglobulin light chain aggregate, e.g., amyloid fibril, in a kidney. In some embodiments, the subject can have one or more immunoglobulin light chain aggregate in a subregion of a kidney, including a glomerulus, a renal capsule, a renal cortex, a renal medulla, a renal pyramid, a renal column, a renal papilla, a minor calyx, a major calyx, a renal pelvis, or a renal hilus. In some embodiments, the subject can have one or more immunoglobulin light chain aggregate, e.g., amyloid fibril, in a tissue of the nervous system. In some embodiments, the subject can have one or more immunoglobulin light chain aggregate, e.g., amyloid fibril, in the brain. In some embodiments, the subject can have one or more immunoglobulin light chain aggregate, e.g., amyloid fibril, in a tissue of the gastrointestinal system. In some embodiments, the subject has one or more immunoglobulin light chain aggregate, e.g., amyloid fibril, in a mouth, a pharynx, an esophagus, a stomach, a small intestine (a duodenum, a jejunum, an ileum), a large intestine (cecum, a colon, a rectum), and/or an anus.

In some embodiments, the method can result in a decrease in the size of the kappa light chain aggregate in a cardiac tissue, a renal tissue, a pulmonary tissue, an intestinal tissue, a gastrointestinal (GI) tissue, a soft tissue, a synovial tissue, a spleen tissue, or an immune tissue compared to a size of the kappa light chain aggregate in a similar tissue prior to the administering.

In some embodiments, the method can result in a decrease in the amount, burden, or size of the kappa light chain aggregate in a cardiac tissue compared to a size of the kappa light chain aggregate in a similar cardiac tissue prior to the administering. In some embodiments, the method can result in a decrease in the size of the kappa light chain aggregate in a renal tissue compared to a size of the kappa light chain aggregate in a similar renal tissue prior to the administering. In some embodiments, the method can result in a decrease in the size of the kappa light chain aggregate in a similar colon tissue compared to a size of the kappa light chain aggregate in a similar colon tissue prior to the administering. In some embodiments, the method can result in a decrease in the amount, burden, or size of the kappa light chain aggregate in a renal tissue compared to an amount, burden, or size of the kappa light chain aggregate in a similar renal tissue prior to the administering. In some embodiments, the method can result in a decrease in the amount, burden, or size of the kappa light chain aggregate in a similar colon tissue compared to an amount, burden, or size of the kappa light chain aggregate in a similar colon tissue prior to the administering.

Certain uses of the anti-human κ light chain antibodies or human κ light chain binding antibody fragments described herein are directed to methods of treating a nephrotic syndrome in a subject in need thereof, comprising administering to the subject an anti-human κ light chain antibody or human κ light chain binding antibody fragment described herein, wherein the nephrotic syndrome comprises one or more immunoglobulin light chain deposits, e.g. Certain uses of the anti-human κ light chain antibodies or human κ light chain binding antibody fragments described herein are directed to methods of treating a hepatomegaly in a subject in need thereof, comprising administering to the subject an anti-human κ light chain antibody or human κ light chain binding antibody fragment described herein, wherein the hepatomegaly comprises one or more immunoglobulin light chain deposit, e.g., one or more amyloid fibril deposit. Certain uses of the anti-human κ light chain antibodies or human κ light chain binding antibody fragments described herein are directed to methods of treating a peripheral neuropathy in a subject in need thereof, comprising administering to the subject an anti-human κ light chain antibody or human κ light chain binding antibody fragment described herein, wherein the peripheral neuropathy comprises one or more immunoglobulin light chain deposit, e.g., one or more amyloid fibril deposit. Certain uses of the anti-human κ light chain antibodies or human κ light chain binding antibody fragments described herein are directed to methods of treating a restrictive cardiomyopathy in a subject in need thereof, comprising administering to the subject an anti-human κ light chain antibody or human κ light chain binding antibody fragment described herein, wherein the restrictive cardiomyopathy comprises one or more immunoglobulin light chain deposit, e.g., one or more amyloid fibril deposit. In some embodiments, the anti-human κ light chain antibodies or human κ light chain binding antibody fragments described herein can be used in a method of treating an amyloidosis (e.g., systemic amyloidosis, a light chain amyloidosis). In some embodiments, the anti-human κ light chain antibodies or human κ light chain binding antibody fragments described herein can be used in a method of treating a light chain amyloidosis. In some embodiments, the anti-human κ light chain antibodies or human κ light chain binding antibody fragments described herein can be used in a method of treating a light chain amyloidosis. In some embodiments, the anti-human κ light chain antibodies or human κ light chain binding antibody fragments described herein can be used in a method of treating a cardiac amyloidosis. In some embodiments, the anti-human κ light chain antibodies or human κ light chain binding antibody fragments described herein can be used in a method of treating a renal amyloidosis.

In some embodiments, administering the anti-human κ light chain antibody or human κ light chain binding antibody fragment to the subject can result in the reduction and/or clearance of one or more immunoglobulin light chain aggregates, e.g., κ amyloid fibril or misfolded κ light chain aggregates, in the subject. In some embodiments, the size of an immunoglobulin light chain aggregate, e.g., amyloid fibril, in the subject can be reduced following administration of the anti-human κ light chain antibody or human κ light chain binding antibody fragment. In some embodiments, administering the anti-human κ light chain antibody or human κ light chain binding antibody fragment to the subject can result in the reduction of misfolded κ light chain aggregates (e.g., amyloidomas) in the subject. In some embodiments, administering the anti-human κ light chain antibody or human κ light chain binding antibody fragment to the subject can result in an increase in phagocytic uptake of the misfolded κ light chain aggregates, a decrease in a number of misfolded κ light chain aggregates, or a decrease in a size of misfolded κ light chain aggregates (e.g., amyloidomas) in the subject compared to a control subject. In some embodiments, administering the anti-human κ light chain antibody or human κ light chain binding antibody fragment to the subject can result in an increase in recruitment of monocytes or macrophages in contact with misfolded κ light chain aggregates (e.g., amyloidomas). In some embodiments, the increase in recruitment of monocytes or macrophages in contact with misfolded κ light chain aggregates comprises an increase in a number of macrophages surrounding or within an amyloidoma of the subject. In some embodiments, the macrophages express F4/80.

In certain embodiments, the antibodies can be administered to a subject in need thereof by any route suitable for the administration of antibody-containing pharmaceutical compositions, such as, for example, subcutaneous, intraperitoneal, intravenous, intramuscular, etc. In certain embodiments, the antibodies can be administered intravenously. In certain embodiments, the antibodies can be administered subcutaneously. In certain embodiments, the antibodies can be administered on a suitable dosage schedule, for example, daily, weekly, twice weekly, monthly, twice monthly, once every two weeks, once every three weeks, or once per month etc. In certain embodiments, the antibodies can be administered once every three weeks. The antibodies can be administered in any therapeutically effective amount. In certain embodiments, therapeutically acceptable amount can be between about 0.1 mg/kg and about 100 mg/kg, between about 1 mg/kg and about 80 mg/kg, between about 1 mg/kg and about 60 mg/kg, between about 1 mg/kg and about 40 mg/kg, between about 1 mg/kg and about 30 mg/kg, between about 10 mg/kg and about 40 mg/kg, or between about 10 mg/kg and about 30 mg/kg. In certain embodiments, therapeutically acceptable amount can be between about 1 mg/kg and about 40 mg/kg. In certain embodiments, therapeutically acceptable amount can be between about 10 mg/kg and about 30 mg/kg. In certain embodiments, therapeutically acceptable amount can be between about 1 mg/kg and about 10 mg/kg. Therapeutically effective amounts include amounts sufficient to ameliorate one or more symptoms associated with the disease or affliction to be treated.

In some embodiments, the control subject can be a subject prior to administration of the anti-human κ light chain antibodies or binding antibody fragments thereof (e.g., a within-subject comparison). In some embodiments, the control subject can be a subject who was not administered the anti-human κ light chain antibodies or binding antibody fragments thereof (e.g., an untreated subject). In some embodiments, the control subject can be a subject who was administered a human isotype control (e.g., human IgG isotype control) or vehicle only.

In some embodiments, the subject can be a mammal. In some embodiments, the subject can be a primate. In some embodiments, the subject can be a human. In some embodiments, the subject can be a non-human primate. In some embodiments, the subject can be a monkey, a chimpanzee, a macaque, a cat, a dog, a sheep, a goat, a chicken, a camel, or a llama.

### Methods of Manufacturing Anti-Human κ Light Chain Antibodies

Provided and described herein are methods of producing or manufacturing the anti-human κ light chain antibodies or human kappa light chain binding fragments provided and described herein.

In some embodiments, the method comprises culturing a cell comprising a nucleic acid encoding the anti-human kappa light chain antibodies or binding fragments thereof. In some embodiments, the cell is cultured under conditions sufficient to allow production and secretion of the anti-human kappa light chain antibodies or binding fragments thereof. In some embodiments, the method comprises culturing a cell comprising a nucleic acid encoding the anti-human kappa light chain antibody or human kappa light chain binding fragment thereof in a cell culture medium under conditions sufficient to allow production and secretion of anti-human kappa light chain antibody or the human kappa light chain binding fragment thereof from the cell. In some embodiments, the method further comprises isolating the anti-human kappa light chain antibody or the human kappa light chain binding fragment thereof from the cell culture medium conditioned by the cell. In some embodiments, the method comprises culturing a cell comprising a nucleic acid encoding the anti-human kappa light chain antibody or human kappa light chain binding fragment thereof in a cell culture medium under conditions sufficient to allow production and secretion of anti-human kappa light chain antibody or the human kappa light chain binding fragment thereof from the cell; and isolating the anti-human kappa light chain antibody or the human kappa light chain binding fragment thereof from the cell culture medium conditioned by the cell.

The nucleic acids encoding the anti-human κ light chain antibodies or human kappa light chain binding fragments of this disclosure can be used to infect, transfect, transform, or otherwise render a suitable cell transgenic for the nucleic acid, thus enabling the production of composite binding molecules for commercial or therapeutic uses. Standard cell lines and methods for the production of antibodies from a large-scale cell culture are known in the art. See e.g., Li et al., “Cell culture processes for monoclonal antibody production.” Mabs. 2010 September-October; 2(5): 466-477.

In certain embodiments, a nucleic acid sequence encodes the anti-human κ light chain antibodies or human kappa light chain binding fragments disclosed herein. In certain embodiments, the polynucleotide sequence encoding the composite binding molecule is operatively coupled to a eukaryotic regulatory sequence. In some embodiments, a cell comprises the nucleic acid sequence.

In some embodiments, a cell comprises a nucleic acid encoding the anti-human κ light chain antibodies or human kappa light chain binding fragments disclosed herein.

In certain embodiments, the cell comprises a prokaryotic cell. In certain embodiments, the prokaryotic cell is an Escherichia coli cell. In certain embodiments, the cell comprises a eukaryotic cell. In certain embodiments, the eukaryotic cell is a Chines Hamster Ovary (CHO) cell, an NS0 murine myeloma cell, a human embryonic kidney cell (e.g., HEK293), or a human PER.C6 cell.

In certain embodiments, described herein is a method of making an anti-human κ light chain antibodies or human kappa light chain binding fragments comprising culturing a cell comprising a nucleic acid encoding the anti-human κ light chain antibodies or binding fragments thereof under conditions in vitro sufficient to allow production and secretion of the composite binding molecules.

In certain embodiments, described herein, is a master cell bank comprising: (a) a mammalian cell line comprising a nucleic acid encoding an antibody described herein integrated at a genomic location; and (b) a cryoprotectant. In certain embodiments, the cryoprotectant can comprise glycerol. In certain embodiments, the master cell bank comprises: (a) a CHO cell line comprising a nucleic acid encoding a composite binding molecule integrated at a genomic location; and (b) a cryoprotectant. In certain embodiments, the cryoprotectant can comprise glycerol. In certain embodiments, the master cell bank can be contained in a suitable vial or container able to withstand freezing by liquid nitrogen.

In some embodiments, the method further comprises incubating a cell, a primary cell, or a cell-line comprising a nucleic acid encoding the anti-human κ light chain antibodies or human kappa light chain binding fragments thereof in a cell culture medium under conditions sufficient to allow for expression and secretion of the anti-human κ light chain antibodies or human kappa light chain binding fragments.

In some embodiments, the method further comprises harvesting the anti-human κ light chain antibodies or human kappa light chain binding fragments from the cell culture medium. The harvesting can further comprise one or more purification steps to remove live cells, cellular debris, non-composite binding molecules proteins or polypeptides, undesired salts, buffers, and medium components. In certain embodiments, the additional purification step(s) include centrifugation, ultracentrifugation, protein A, protein G, protein A/G, or protein L purification, and/or ion exchange chromatography.

### Pharmaceutically Acceptable Excipients, Carriers, and Diluents

In certain embodiments the anti-kappa light chain antibodies of the current disclosure are included in a pharmaceutical composition comprising one or more pharmaceutically acceptable excipients, carriers, and diluents. Pharmaceutically acceptable excipients, carriers and diluents can be included to increase shelf-life, stability, or the administrability of the antibody. Such compounds include salts, pH buffers, detergents, anti-coagulants, and preservatives. In certain embodiments, the antibodies of the current disclosure can be administered suspended in a sterile solution. In certain embodiments, the solution can comprise about 0.9% NaCl. In certain embodiments, the solution can comprise about 5.0% dextrose. In certain embodiments, the solution further can comprise one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine or arginine; antioxidants, for example, ascorbic acid, methionine; or chelating agents, for example, EDTA or EGTA. In certain embodiments, the pharmaceutical composition can comprise a nucleic acid sequence encoding the anti-human kappa light chain antibody or human kappa light chain binding fragment thereof. In some embodiments, the pharmaceutical composition comprises: (i) a nucleic acid sequence encoding the anti-human kappa light chain antibody or human kappa light chain binding fragment thereof; and (ii) a vector. The vector can, for example, encapsulate the nucleic acid comprising any nucleic acid sequence encoding the anti-human kappa light chain antibody or human kappa light chain binding fragment thereof, as previously described. Alternatively, the vector can conjugate with a nucleic acid comprising a nucleic acid sequence encoding an anti-human kappa light chain antibody or human kappa light chain binding fragment thereof previously provided in this disclosure. The nucleic acid of this disclosure can be encapsulated or coupled to a vector. In some embodiments, the vector can be configured to deliver any one or more of the nucleic acids described herein to a subject. In some cases, the vector can be a non-viral vector. In some embodiments, the non-viral vector can be a lipid nanoparticle (LNP), a liposome, a cationic lipid, a polymer, an inorganic nanoparticle, an organic nanoparticle, an extracellular vesicle (e.g., a microvesicle or an exosome), or a peptide. In some embodiments, the vector can be a viral vector. In some cases, the viral vector can be an adenoviral vector, an associated-adeno viral vector, a lentiviral vector, a retroviral vector, or a herpes simplex viral vector.

In some embodiments, the vector can comprise a first nucleic acid sequence encoding a heavy chain variable region comprising an amino acid sequence as set forth in SEQ ID NO: 1, or encoding a heavy chain variable region comprising an H-CDR1, a H-CDR2, and a H-CDR3 as set forth in the amino acid sequence of SEQ ID NO: 1, and/or a second nucleic acid encoding a light chain variable region comprising an amino acid sequence as set forth in SEQ ID NO: 5, or encoding a light chain variable region comprising an L-CDR1, an L-CDR2, and an L-CDR3 as set forth in the amino acid sequence of SEQ ID NO: 5.

In some embodiments, the vector can comprise a first nucleic acid sequence encoding a heavy chain variable region comprising an amino acid sequence as set forth in SEQ ID NO: 9, or encoding a heavy chain variable region comprising an H-CDR1, a H-CDR2, and a H-CDR3 as set forth in the amino acid sequence of SEQ ID NO: 9, and/or a second nucleic acid encoding a light chain variable region comprising an amino acid sequence as set forth in SEQ ID NO: 13, or encoding a light chain variable region comprising an L-CDR1, an L-CDR2, and an L-CDR3 as set forth in the amino acid sequence of SEQ ID NO: 13.

In some embodiments, a first nucleic acid sequence encoding a heavy chain variable region comprising an amino acid sequence as set forth in SEQ ID NO: 17, or encoding a heavy chain variable region comprising an H-CDR1, a H-CDR2, and a H-CDR3 as set forth in the amino acid sequence of SEQ ID NO: 17, and/or a second nucleic acid encoding a light chain variable region comprising an amino acid sequence as set forth in SEQ ID NO: 21, or encoding a light chain variable region comprising an L-CDR1, an L-CDR2, and an L-CDR3 as set forth in the amino acid sequence of SEQ ID NO: 21.

In some embodiments, the vector can comprise a first nucleic acid sequence encoding a heavy chain variable region comprising an amino acid sequence as set forth in SEQ ID NO: 25, or encoding a heavy chain variable region comprising an H-CDR1, a H-CDR2, and a H-CDR3 as set forth in the amino acid sequence of SEQ ID NO: 25, and/or a second nucleic acid encoding a light chain variable region comprising an amino acid sequence as set forth in SEQ ID NO: 29, or encoding a light chain variable region comprising an L-CDR1, an L-CDR2, and an L-CDR3 as set forth in the amino acid sequence of SEQ ID NO: 29.

In certain embodiments, the antibodies of the current disclosure can be shipped/stored lyophilized and reconstituted before administration. In certain embodiments, lyophilized antibody formulations comprise a bulking agent such as, mannitol, sorbitol, sucrose, trehalose, dextran 40, or combinations thereof. The lyophilized formulation can be contained in a vial comprised of glass or other suitable non-reactive material. The antibodies when formulated, whether reconstituted or not, can be buffered at a certain pH, generally less than 7.0. In certain embodiments, the pH can be between 4.5 and 7.0, 4.5 and 6.5, 4.5 and 6.0, 4.5 and 5.5, 4.5 and 5.0, or 5.0 and 6.0.

Also described herein are kits comprising one or more of the anti-human kappa light chain antibodies or human kappa light chain binding fragments described herein in a suitable container and one or more additional components selected from: instructions for use; a diluent, an excipient, a carrier, and a device for administration. In some embodiments, the kit can comprise one or more of the anti-human kappa light chain antibodies or human kappa light chain binding fragments provided and described herein. In some embodiments, the kit can comprise a syringe, a vial, or any receptacle suitable receptable or container for the anti-K light chain antibody or κ light chain binding fragment thereof described herein. In some embodiments, the kit can comprise a set of instructions for administering the anti-K light chain antibody or κ light chain binding fragment thereof. The instructions can instruct a physician or a subject how to administer the anti-K light chain antibody or κ light chain binding fragment thereof. In some cases, the instructions can be provided for a subject to self-administer the anti-K light chain antibody or κ light chain binding fragment thereof.

In certain embodiments, described herein is a method of preparing a kappa light chain amyloidosis treatment comprising admixing one or more pharmaceutically acceptable excipients, carriers, or diluents and an antibody of the current disclosure. In certain embodiments, described herein is a method of preparing a cancer treatment for storage or shipping comprising lyophilizing one or more antibodies of the current disclosure.

### DEFINITIONS

An antibody is used in the broadest sense and generally encompasses and/or refers to various antibody structures, including but not limited to monoclonal antibodies, polyclonal antibodies, multispecific antibodies (e.g., bispecific antibodies), and antibody fragments so long as they exhibit the desired antigen-binding activity. In some embodiments, an antibody or antibodies include intact antibodies and functional (antigen-binding) antibody fragments, including fragment antigen binding (Fab) fragments, F(ab′)₂ fragments, Fab′ fragments, Fv fragments, recombinant IgG (rIgG) fragments, single chain antibody fragments, including single chain variable fragments (sFv or scFv), and single domain antibodies (e.g., sdAb, sdFv, nanobody) fragments. In some embodiments, an antibody or antibodies include genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. In some embodiments, an antibody or antibodies encompass functional antibody fragments. In some embodiments, an antibody or antibodies encompasses intact or full-length antibodies, including antibodies of any class or sub-class, including IgG and sub-classes thereof, IgM, IgE, IgA, and IgD. The antibody can comprise a human IgG1 constant region. The antibody can comprise a human IgG4 constant region. In some embodiments, an antibody or antibodies include, but is not limited to, full-length and native antibodies, as well as fragments and portion thereof retaining the binding specificities thereof, such as any specific binding portion thereof including those having any number of, immunoglobulin classes and/or isotypes (e.g., IgGl, IgG2, IgG3, IgG4, IgM, IgA, IgD, IgE and IgM); and biologically relevant (antigen-binding) fragments or specific binding portions thereof, including but not limited to Fab, F(ab′)₂, Fv, and scFv (single chain or related entity). A monoclonal antibody is generally one within a composition of substantially homogeneous antibodies; thus, any individual antibodies comprised within the monoclonal antibody composition are identical except for possible naturally occurring mutations that can be present within a composition in minor amounts. A monoclonal antibody can comprise a human IgG1 constant region or a human IgG4 constant region.

A native antibody generally encompasses and/or refers to naturally occurring immunoglobulin molecules with varying structures. For example, native IgG antibodies are heterotetrameric glycoproteins of about 150,000 Daltons, composed of two identical light chains and two identical heavy chains that are disulfide bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody can be assigned to one of two types, called kappa (κ) and lambda (λ), based on the amino acid sequence of its constant domain.

A full-length antibody, intact antibody, and whole antibody are interchangeable, and generally include and/or refer to an antibody having a structure substantially similar to a native antibody structure or having heavy chains that contain an Fc region as defined herein.

A human consensus framework generally encompasses and/or refers to a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In some embodiments, for the VL, the subgroup is subgroup kappa I as in Kabat et al., supra. In some embodiments, for the VH, the subgroup is subgroup III as in Kabat et al., supra. A humanized antibody encompasses and refers to a chimeric antibody comprising amino acid residues from non-human HVRs and amino acid residues from human framework regions (FRs). In some embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally encompasses at least a portion of an antibody constant region derived from a human antibody. A “humanized form” of an antibody, e.g., a non-human antibody, refers to an antibody that has undergone humanization.

A complementarity determining region or CDR, which are synonymous with hypervariable region or HVR, generally include and/or refer to regions of an antibody which are hypervariable in sequence and/or form structurally defined loops (hypervariable loops) and/or contain the antigen-contacting residues (antigen contacts). In some embodiments, complementarity determining regions or CDRs generally include and refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3).

A framework region or FR generally includes and/or refers to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). In some embodiments, the framework regions are defined by the non-CDR sequences of a variable region sequence. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Pluckthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg N R and Rees A R, “WAM: an improved algorithm for modelling antibodies on the WEB,” Protein Eng. 2000 December; 13(12):819-24 (“AbM” numbering scheme. In certain embodiments, the CDRs of the antibodies described herein can be defined by a method selected from Kabat, Chothia, IMGT, Aho, AbM, or combinations thereof. In some embodiments, the CDRs and FRs are defined by and/or according to a Kabat numbering scheme. In some embodiments, the CDRs and FRs are defined by and/or according to a Chothia numbering scheme. In some embodiments, the CDRs and FRs are defined by and/or according to a IMGT numbering scheme. In some embodiments, the CDRs and FRs are defined by and/or according to an EU numbering scheme.

The boundaries of a given CDR or FR, in certain instances, vary depending on the scheme used for identification. For example, the Kabat scheme is based on structural alignments, while the Chothia scheme is based on structural information.

Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

Fc region generally encompasses and/or refers to a C-terminal region of an immunoglobulin heavy chain that contains at least a portion of the constant region. The term includes native sequence Fc regions and variant Fc regions. In some embodiments, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

A human antibody generally encompasses and/or refers to an antibody which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

An acceptor human framework region generally encompasses and/or refers to a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework can comprise the same amino acid sequence thereof, or it can contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

A variable region or variable domain generally encompasses and/or refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs (See e.g., Kindt et al. Kuby Immunology, 6th ed., W.H. Freeman and Co., page 91(2007)). A single V_{H} or V_{L} domain can be sufficient to confer antigen-binding specificity. Furthermore, antibodies that bind a particular antigen can be isolated using a V_{H} or V_{L} domain from an antibody that binds the antigen to screen a library of complementary V_{L} or V_{H} domains, respectively (See e.g., Portolano et al., J. Immunol. 150:880-887 (1993); Clarkson et al., Nature 352:624-628 (1991)).

Affinity generally encompasses and/or refers to the strength of the sum total of noncovalent interactions between a single binding site of a molecule (e.g., an antibody) and its binding partner (e.g., an antigen). Unless indicated otherwise, as used herein, binding affinity generally encompasses and refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary embodiments for measuring binding affinity are described throughout.

An affinity matured antibody generally encompasses and/or refers to an antibody with one or more alterations in one or more hypervariable regions (HVRs), compared to a parent antibody which does not possess such alterations, such alterations resulting in an improvement in the affinity of the antibody for antigen.

Binding and a determination of binding (e.g., to an epitope comprising SEQ ID NO: 51) can be readily determined by methods known within the art (e.g., ELISA, surface plasmon resonance, bio-layer interferometry, isothermal calorimetry, etc.). In some embodiments, binding is determined by ELISA. In some embodiments, binding comprising a K_{D} less than, e.g., 10⁻⁵ M (10 uM) as measured by surface plasmon resonance, bio-layer interferometry, or isothermal calorimetry. In some embodiments, binding comprising a K_{D} less than, e.g., 10⁻⁶ M (1 uM) surface plasmon resonance, bio-layer interferometry, or isothermal calorimetry. In some embodiments, binding comprising a K_{D} less than, e.g., 10⁻⁷ M (100 nM) surface plasmon resonance, bio-layer interferometry, or isothermal calorimetry.

A chimeric antibody generally encompasses and/or refers to an antibody in which a portion of the heavy and/or light chain is derived from a particular source or species, while the remainder of the heavy and/or light chain is derived from a different source or species. 1 The “class” of an antibody refers to the type of constant domain or constant region possessed by its heavy chain. There are five major classes of antibodies: IgA, IgD, IgE, IgG, and IgM, and several of these can be further divided into subclasses (isotypes), e.g., IgG₁, IgG₂, IgG₃, IgG₄, IgA₁, and IgA₂. The heavy chain constant domains that correspond to the different classes of immunoglobulins are called a, d, e, g, and m, respectively.

A monoclonal antibody generally encompasses and/or refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present within a composition in minor amounts. In contrast to polyclonal antibody preparations, which typically include different antibodies directed against different determinants (epitopes), each monoclonal antibody of a monoclonal antibody preparation is directed against a single determinant on an antigen. In some embodiments, the modifier monoclonal indicates the character of the antibody as being obtained from a substantially homogeneous population of antibodies and is not to be construed as requiring production of the antibody by any particular method. For example, the monoclonal antibodies to be used in accordance with the present within a composition disclosure can be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

An isolated antibody generally encompasses and/or refers to an antibody that has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC).

In certain embodiments, the antibody comprises one or more naturally occurring amino acids. In certain embodiments, the antibody consists of naturally occurring amino acids. As used herein, naturally occurring amino acids include and/or refer to amino acids which are generally found in nature and are not manipulated by man. In certain instances, naturally occurring includes and/or further refers to the 20 conventional amino acids: alanine (A or Ala), cysteine (C or Cys), aspartic acid (D or Asp), glutamic acid (E or Glu), phenylalanine (F or Phe), glycine (G or Gly), histidine (H or His), isoleucine (I or Ile), lysine (K or Lys), leucine (L or Leu), methionine (M or Met), asparagine (N or Asn), proline (P or Pro), glutamine (Q or Gln), arginine (R or Arg), serine (S or Ser), threonine (T or Thr), valine (V or Val), tryptophan (W or Trp), and tyrosine (Y or Tyr).

In some embodiments, the antibody comprises a variant sequence of the antibody. In certain instances, amino acid substitutions can be made in the sequence of any of the antibodies described herein, without necessarily decreasing or ablating its activity (as measured by, e.g., the binding or functional assays described herein). Accordingly, in some embodiments, the variant sequence comprises one or more amino acid substitutions (e.g., within the variable region or within one or more CDRs). In some embodiments, the variant sequence comprises one or more substitutions in one or more CDRs. In certain embodiments, the variant sequence comprises one amino acid substitution. In certain embodiments, the variant sequence comprises two amino acid substitutions. In certain embodiments, the variant sequence comprises three amino acid substitutions. In certain instances, substitutions include conservative substitutions (e.g., substitutions with amino acids of comparable chemical characteristics). In certain instances, a non-polar amino acid can be substituted and replaced with another non-polar amino acid, wherein non-polar amino acids include alanine, leucine, isoleucine, valine, glycine, proline, phenylalanine, tryptophan and methionine. In certain instances, a neutrally charged polar amino acids can be substituted and replaced with another neutrally charged polar amino acid, wherein neutrally charged polar amino acids include serine, threonine, cysteine, tyrosine, asparagine, and glutamine. In certain instances, a positively charged amino acid can be substituted and replaced with another positively charged amino acid, wherein positively charged amino acids include arginine, lysine and histidine. In certain instances, a negatively charged amino acid can be substituted and replaced with another negatively charged amino acid, wherein negatively charged amino acids include aspartic acid and glutamic acid. Examples of amino acid substitutions also include substituting an L-amino acid for its corresponding D-amino acid, substituting cysteine for homocysteine or other non-natural amino acids.

In certain embodiments, the antibody comprises one or more non-natural amino acids. In certain embodiments, the antibody consists of non-natural amino acids. As used herein, non-natural amino acids and/or unnatural amino acids include and/or refer to amino acid structures that cannot be generated biosynthetically in any organism using unmodified or modified genes from any organism. For example, these include, but are not limited to, modified amino acids and/or amino acid analogues that are not one of the 20 naturally occurring amino acids (e.g., non-natural side chain variant sequence amino acids), D-amino acids, homo amino acids, beta-homo amino acids, N-methyl amino acids, alpha-methyl amino acids, or. By way of further example, non-natural amino acids also include 4-Benzoylphenylalanine (Bpa), Aminobenzoic Acid (Abz), Aminobutyric Acid (Abu), Aminohexanoic Acid (Ahx), Aminoisobutyric Acid (Aib), Citrulline (Cit), Diaminobutyric Acid (Dab), Diaminopropanoic Acid (Dap), Diaminopropionic Acid (Dap), Gamma-Carboxyglutamic Acid (Gla), Homoalanine (Hala), Homoarginine (Harg), Homoasparagine (Hasn), Homoaspartic Acid (Hasp), Homocysteine (Hcys), Homoglutamic Acid (Hglu), Homoglutamine (Hgln), Homoisoleucine (Hile), Homoleucine (Hleu), Homomethionine (Hmet), Homophenylalanine (Hphe), Homoserine (Hser), Homotyrosine (Htyr), Homovaline (Hval), Hydroxyproline (Hyp), Isonipecotic Acid (Inp), N aphthylalanine (Nal), Nipecotic Acid (Nip), Norleucine (Nle), Norvaline (Nva), Octahydroindole-2-carboxylic Acid (Oic), Penicillamine (Pen), Phenylglycine (Phg), Pyroglutamic Acid (Pyr), Sarcosine (Sar), tButylglycine (Tle), and Tetrahydro-isoquinoline-3-carboxylic Acid (Tic). Such non-natural amino acid residues can be introduced by substitution of naturally occurring amino acids, and/or by insertion of non-natural amino acids into the naturally occurring antibody sequence. A non-natural amino acid residue also can be incorporated such that a desired functionality is imparted to the apelin molecule, for example, the ability to link a functional moiety (e.g., PEG).

A stable formulation refers to and/or encompasses a formulation wherein the protein (e.g., antibody) therein essentially retains its physical stability and/or chemical stability and/or biological activity upon storage at an intended storage temperature, e.g., 2-8 °C In some embodiments, the formulation essentially retains its physical and chemical stability, as well as its biological activity upon storage. A storage period can generally be selected based on the intended shelf-life of the formulation. Furthermore, the formulation is generally stable following freezing (to, e.g., −20 °C) and thawing of the formulation, for example following 1 or more cycles of freezing and thawing. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993), for example. Stability can be measured at a selected temperature for a selected time period. Stability can be evaluated qualitatively and/or quantitatively in a variety of different ways, including evaluation of aggregate formation (for example using size exclusion chromatography, by measuring turbidity, and/or by visual inspection); by assessing charge heterogeneity using cation exchange chromatography or capillary zone electrophoresis; SDS-PAGE analysis to compare reduced and intact antibody; evaluating biological activity or antigen binding function of the antibody; and the methods described herein. Instability can involve any one or more of: aggregation, deamidation (e.g., Asn deamidation), oxidation (e.g. Met oxidation), isomerization (e.g. Asp isomeriation), clipping/hydrolysis/fragmentation (e.g. hinge region fragmentation), succinimide formation, unpaired cysteine(s), etc.

A pharmaceutically acceptable carrier generally encompasses and/or refers to an ingredient in a pharmaceutical formulation, other than an active ingredient, which is nontoxic to a subject. A pharmaceutically acceptable carrier encompasses, but is not limited to, a buffer, excipient, stabilizer, or preservative.

A polypeptide or protein are used interchangeably and generally encompass and/or refer to a polymer of amino acid residues and are not limited to a minimum length. Polypeptides, including the provided antibodies and antibody chains and other peptides, e.g., linkers and binding peptides, can include amino acid residues including natural and/or non-natural amino acid residues. The terms also include post-expression modifications of the polypeptide, for example, glycosylation, sialylation, acetylation, phosphorylation, and the like. In some embodiments, the polypeptides can contain modifications with respect to a native or natural sequence, as long as the protein maintains the desired activity. These modifications can be deliberate, as through site-directed mutagenesis, or can be accidental, such as through mutations of hosts which produce the proteins or errors due to PCR amplification. In some embodiments, the anti-κ light chain antibody or κ light chain binding fragment thereof can have a heavy chain variable region and a light chain variable region that are coupled directly or indirectly. In some embodiments, the heavy chain variable region and the light chain variable region can be coupled via a peptide, e.g., a peptide linker.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

As used herein the term individual, patient, or subject generally includes and/or refers to individuals diagnosed with, suspected of being afflicted with, or at-risk of developing at least one disease, condition, or status for which the described compositions and method are useful for treating. In certain embodiments, the individual is a mammal. In certain embodiments, the mammal is a mouse, rat, rabbit, dog, cat, horse, cow, sheep, pig, goat, llama, alpaca, or yak. In certain embodiments, the individual is a human.

The antibodies described herein can be encoded by a nucleic acid. A nucleic acid is a type of polynucleotide comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In the expression vectors regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a genomic region. In certain embodiments, the expression vector is a plasmid. In certain embodiments, the expression vector is a lentivirus, adenovirus, or adeno-associated virus. In certain embodiments, the expression vector is an adenovirus. In certain embodiments, the expression vector is an adeno-associated virus. In certain embodiments, the expression vector is a lentivirus.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). Percent homology of sequences can be determined using the most recent version of BLAST, as of the filing date of this application.

The nucleic acids encoding the antibodies described herein can be used to infect, transfect, transform, or otherwise render a suitable cell transgenic for the nucleic acid, thus enabling the production of antibodies for commercial or therapeutic uses. Standard cell lines and methods for the production of antibodies from a large-scale cell culture are known in the art. See e.g., Li et al., “Cell culture processes for monoclonal antibody production.” Mabs. 2010 September-October; 2(5): 466-477. In certain embodiments, the cell is a Eukaryotic cell. In certain embodiments, the Eukaryotic cell is a mammalian cell. In certain embodiments, the mammalian cell is a cell line useful for producing antibodies is a Chines Hamster Ovary cell (CHO) cell, an NS0 murine myeloma cell, or a PER.C6® cell. In certain embodiments, the nucleic acid encoding the antibody is integrated into a genomic locus of a cell useful for producing antibodies. In certain embodiments, described herein is a method of making an antibody comprising culturing a cell comprising a nucleic acid encoding an antibody under conditions in vitro sufficient to allow production and secretion of said antibody.

In certain embodiments, described herein, is a master cell bank comprising: (a) a mammalian cell line comprising a nucleic acid encoding an antibody described herein integrated at a genomic location; and (b) a cryoprotectant. In certain embodiments, the cryoprotectant comprises glycerol or DMSO. In certain embodiments, the master cell bank comprises: (a) a CHO cell line comprising a nucleic acid encoding an antibody of the disclosure; and (b) a cryoprotectant. In certain embodiments, the cryoprotectant comprises glycerol or DMSO. In certain embodiments, the master cell bank is contained in a suitable vial or container able to withstand freezing by liquid nitrogen.

Also described herein are methods of making an antibody described herein. Such methods comprise incubating a cell or cell-line comprising a nucleic acid encoding the antibody in a cell culture medium under conditions sufficient to allow for expression and secretion of the antibody, and further harvesting the antibody from the cell culture medium. The harvesting can further comprise one or more purification steps to remove live cells, cellular debris, non-antibody proteins or polypeptides, undesired salts, buffers, and medium components. In certain embodiments, the additional purification step(s) include centrifugation, ultracentrifugation, protein A, protein G, protein A/G, or protein L purification, and/or ion exchange chromatography.

“Treat,” “treatment,” or “treating,” as used herein refers to, e.g., a deliberate intervention to a physiological disease state resulting in the reduction in severity of a disease or condition; the reduction in the duration of a condition course; the amelioration or elimination of one or more symptoms associated with a disease or condition; or the provision of beneficial effects to a subject with a disease or condition. Treatment does not require curing the underlying disease or condition.

A “therapeutically effective amount,” “effective dose,” “effective amount,” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, “pharmaceutically acceptable” with reference to a carrier” “excipient” or “diluent” includes any and all solvents, dispersion media, coatings, antibacterial and antifungal agents, isotonic and absorption delaying agents, and the like that are physiologically compatible. In some aspects, the carrier is suitable for intravenous, intramuscular, subcutaneous, parenteral, spinal or epidermal administration (e.g., by injection or infusion). Depending on the route of administration, the active compound, i.e., antibody, can be coated in a material to protect the compound from the action of acids and other natural conditions that can inactivate the compound.

The pharmaceutical compounds described herein can include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66: 1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

As used herein, treatment or treating generally include and/or refer to a pharmaceutical or other intervention regimen used for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made. Skilled artisans will recognize that given a population of potential individuals for treatment not all will respond or respond equally to the treatment. Such individuals are considered treated.

In some embodiments, treating misfolded κ light chains and/or treating κ light chain amyloid deposits or treating κ light chain aggregates can be used interchangeably herein. In some embodiments, treating misfolded κ light chains includes treating a disorder characterized by misfolded κ light chains. In some embodiments, treating misfolded κ light chains comprises decreasing and/or reducing and/or inhibiting κ light chain aggregates (e.g., by measuring freely circulating light chains) or a symptom thereof. In some embodiments, treating misfolded κ light chains comprises promoting the clearance of treating misfolded κ light chains by monocytic cells and/or phagocytotic cells (e.g., by measuring freely circulating light chains).

As used herein, monocyte or monocyte cell refers and/or encompasses to a type of leukocyte, or white blood cell. Monocytes are the largest class of leukocyte and can differentiate into, e.g., macrophages. As a part of the vertebrate innate immune system monocytes also influence the process of adaptive immunity. Monocytes can generally be classified three types of monocytes in human blood: 1) The classical monocyte may be characterized by high level expression of the CD14 cell surface receptor (CD14⁺⁺CD16⁻ monocyte); 2) The non-classical monocyte shows low level expression of CD14 and additional co-expression of the CD16 receptor (CD14⁺CD16⁺⁺ monocyte); and 3) The intermediate monocyte with high level expression of CD14 and low level expression of CD16 (CD14⁺⁺CD16⁺ monocytes).

As used herein, a macrophage generally refers to and/or encompasses a type of white blood cell of the immune system, that in a process called phagocytosis, engulfs and digests cellular debris, foreign substances, microbes, cancer cells, and anything else that does not have the type of proteins specific to healthy body cells on its surface.

Beyond increasing inflammation and stimulating the immune system, macrophages can also play an important anti-inflammatory role and can decrease immune reactions through the release of cytokines

As used herein, a phagocytes generally refers to and/or encompasses a cell that is capable of phagocytosis and include both professional and non-professional phagocytes.

As used herein, amyloidosis generally refers to and/or encompasses a disease or condition characterized by the misfolding of free immunoglobulin light chains (LC) (e.g., kappa LCs) which can deposit as light chain amyloid (AL) in the heart, kidneys, or liver. The condition is typically caused by an underlying plasma cell dyscrasia that results in plasma cells secreting a large excess of free immunoglobulin kappa (κ) and/or lambda (λ) light chains into circulation. In certain instances, the immunoglobulin kappa (κ) and/or lambda (λ) light chains in circulation are misfolded, prone to aggregation and/or forming amyloid deposits, or aggregated Primary amyloidosis or AL amyloidosis generally refer to and/or encompass the most common type of systemic amyloidosis, with an incidence of 9-14 people per million per year in the Western world.

As used herein, a plasma cell dyscrasia generally refers to and/or encompasses a proliferative plasma cell disorder under which a single, aberrant plasma cell clone (a terminally differentiated B cell) undergoes unregulated expansion and produces above-normal amounts of light chain protein (e.g., misfolded LC protein). Plasma cell dyscrasias can be classified as either benign, as seen in monoclonal gammopathies of unknown significance (MGUS), or malignant, as in multiple myeloma (MM) and plasma cell leukemia (PCL). Regardless of the type of dyscrasia involved, these secreted light chains can undergo misfolding events that render them prone to aggregate and accumulate as amyloid in vital organs including the heart, kidneys and nervous system. Such infiltration can result in nephrotic syndrome, hepatomegaly, peripheral neuropathy, restrictive cardiomyopathy, and ultimately death.

As used herein, the words “comprising” (and any form of comprising, such as comprise and comprises), having (and any form of having, such as have and has), including (and any form of including, such as include and includes) or containing (and any form of containing, such as contain and contains), are inclusive or open-ended and do not exclude additional, unrecited elements or process steps. As also used herein, in any instance or embodiment described herein, comprising may be replaced with consisting essentially of and/or consisting of: used herein, in any instance or embodiment described herein, comprises may be replaced with consists essentially of and/or consists of.

As used herein, the term “about” in the context of a given value or range includes and/or refers to a value or range that is within 20%, within 10%, and/or within 5% of the given value or range.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” can apply to each of the numerical values in that series of numerical values. For example, less than or equal to 3, 2, or 1 is equivalent to less than or equal to 3, less than or equal to 2, or less than or equal to 1.

Throughout this application, various cases may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

“Optional” or “optionally” means that the subsequently described event or circumstance may or may not occur, and that the description includes instances where said event or circumstance occurs and instances where it does not.

“Exemplary” means “an example of” and is not intended to convey an indication of a preferred or ideal aspect. “Such as” is not used in a restrictive sense, but for explanatory purposes.

As used herein, the terms “essentially” or “substantially,” when describing a relative value, a relative amount or a relative degree between two subjects, generally refers to within 80%, 81%, 82%, 83%, 84%, 85%, 86%, 87%, 88%, 89% 90%, 91%, 92%, 93%, 94%, 95%, 96%, 97%, 98%, 99%, 100%, 101%, 102%, 103%, 104%, 105%, 106%, 107%, 108%, 109%, or 110% of each other in value, amount or degree.

The terms “increased”, “increasing”, or “increase” are used herein to generally mean an increase by a statically significant amount. In some cases, the terms “increased,” or “increase,” mean an increase of at least 10% as compared to a reference level, for example an increase of at least 10%, at least 20%, or at least 30%, or at least 40%, or at least 50%, or at least 60%, or at least 70%, or at least 80%, or at least 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms, “decreased”, “decreasing”, or “decrease” are used herein generally to mean a decrease by a statistically significant amount. In some cases, “decreased” or “decrease” means a reduction by at least 10% as compared to a reference level, for example a decrease by at least 20%, or at least 30%, or at least 40%, or at least 50%, or at least 60%, or at least 70%, or at least 80%, or at least 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

As used herein, the term and/or is to be taken as specific disclosure of each of the two specified features or components with or without the other. For example, A and/or B is to be taken as specific disclosure of each of (i) A, (ii) B and (iii) A and B, just as if each were set out individually herein.

### EMBODIMENTS

Embodiment 1. An anti-human kappa light chain antibody or human kappa light chain binding fragment thereof comprising:
   a. a heavy chain complementarity determining region 1 (H-CDR1) comprising an amino acid sequence set forth in SEQ ID NO: 2;
   b. a heavy chain complementarity determining region 2 (H-CDR2) comprising an amino acid sequence set forth in SEQ ID NO: 3;
   c. a heavy chain complementarity determining region 3 (H-CDR3) comprising an amino acid sequence set forth in SEQ ID NO: 4;
   d. a light chain complementarity determining region 1 (L-CDR1) comprising an amino acid sequence set forth in SEQ ID NO: 6;
   e. a light chain complementarity determining region 2 (L-CDR2) comprising an amino acid sequence set forth in SEQ ID NO: 7; and
   f. a light chain complementarity determining region 3 (L-CDR3) comprising an amino acid sequence set forth in SEQ ID NO: 8.
Embodiment 2. The anti-human kappa light chain antibody or human kappa light chain binding fragment thereof of embodiment 1, further comprising:
   a. a heavy chain variable region comprising an amino acid sequence of SEQ ID NO: 1; and
   b. a light chain variable region comprising an amino acid sequence of SEQ ID NO: 5.
Embodiment 3. An anti-human kappa light chain antibody or human kappa light chain binding fragment thereof comprising:
   a. the heavy chain variable region comprising an amino acid sequence of SEQ ID NO: 1; and
   b. a light chain variable region comprising an amino acid sequence of SEQ ID NO: 5.
Embodiment 4. The anti-human kappa light chain antibody or human kappa light chain binding fragment thereof of embodiment 1, wherein:
   a. the heavy chain variable region comprises the amino acid sequence of SEQ ID NO: 1; or
   b. the light chain variable region comprises the amino acid sequence of SEQ ID NO: 5.
Embodiment 5. An anti-human kappa light chain antibody or human kappa light chain binding fragment thereof comprising:
   a. a heavy chain complementarity determining region 1 (H-CDR1) comprising an amino acid sequence set forth in SEQ ID NO: 10;
   b. a heavy chain complementarity determining region 2 (H-CDR2) comprising an amino acid sequence set forth in SEQ ID NO:11;
   c. a heavy chain complementarity determining region 3 (H-CDR3) comprising an amino acid sequence set forth in SEQ ID NO: 12;
   d. a light chain complementarity determining region 1 (L-CDR1) comprising an amino acid sequence set forth in SEQ ID NO: 14;
   e. a light chain complementarity determining region 2 (L-CDR2) comprising an amino acid sequence set forth in SEQ ID NO: 15; and
   f. a light chain complementarity determining region 3 (L-CDR3) comprising an amino acid sequence set forth in SEQ ID NO: 16.
Embodiment 6. The anti-human kappa light chain antibody or human kappa light chain binding fragment thereof of embodiment 5, further comprising:
   a. a heavy chain variable region comprising an amino acid sequence of SEQ ID NO: 9; and
   b. a light chain variable region comprising an amino acid sequence of SEQ ID NO: 13.
Embodiment 7. An anti-human kappa light chain antibody or human kappa light chain binding fragment thereof comprising:
   a. the heavy chain variable region comprising an amino acid sequence of SEQ ID NO: 9; and
   b. the light chain variable region comprising an amino acid sequence of SEQ ID NO: 13.
Embodiment 8. The anti-human kappa light chain antibody or human kappa light chain binding fragment thereof of embodiment 5, wherein:
   a. the heavy chain variable region comprises an amino acid sequence of SEQ ID NO: 9; or
   b. the light chain variable region comprises an amino acid sequence of SEQ ID NO: 13.
Embodiment 9. An anti-human kappa light chain antibody or human kappa light chain binding fragment thereof comprising:
   a. a heavy chain complementarity determining region 1 (H-CDR1) comprising an amino acid sequence set forth in SEQ ID NO: 18;
   b. a heavy chain complementarity determining region 2 (H-CDR2) comprising an amino acid sequence set forth in SEQ ID NO: 19;
   c. a heavy chain complementarity determining region 3 (H-CDR3) comprising an amino acid sequence set forth in SEQ ID NO: 20;
   d. a light chain complementarity determining region 1 (L-CDR1) comprising an amino acid sequence set forth in SEQ ID NO: 22;
   e. a light chain complementarity determining region 2 (L-CDR2) comprising an amino acid sequence set forth in SEQ ID NO: 23; and
   f. a light chain complementarity determining region 3 (L-CDR3) comprising an amino acid sequence set forth in SEQ ID NO: 24.
Embodiment 10. The anti-human kappa light chain antibody or human kappa light chain binding fragment thereof of embodiment 9, further comprising:
   a. a heavy chain variable region comprising an amino acid sequence of SEQ ID NO: 17; and
   b. a light chain variable region comprising an amino acid sequence of SEQ ID NO: 21.
Embodiment 11. An anti-human kappa light chain antibody or human kappa light chain binding fragment thereof comprising:
   a. the heavy chain variable region comprising an amino acid sequence of SEQ ID NO: 17; and
   b. the light chain variable region comprising an amino acid sequence of SEQ ID NO: 21.
Embodiment 12. The anti-human kappa light chain antibody or human kappa light chain binding fragment thereof of embodiment 9, wherein:
   a. the heavy chain variable region comprises an amino acid sequence of SEQ ID NO: 17; or
   b. the light chain variable region comprises an amino acid sequence of SEQ ID NO: 21.
Embodiment 13. An anti-human kappa light chain antibody or human kappa light chain binding fragment thereof comprising:
   a. a heavy chain complementarity determining region 1 (H-CDR1) comprising an amino acid sequence set forth in SEQ ID NO: 26;
   b. a heavy chain complementarity determining region 2 (H-CDR2) comprising an amino acid sequence set forth in SEQ ID NO: 27;
   c. a heavy chain complementarity determining region 3 (H-CDR3) comprising an amino acid sequence set forth in SEQ ID NO: 28;
   d. a light chain complementarity determining region 1 (L-CDR1) comprising an amino acid sequence set forth in SEQ ID NO: 30;
   e. a light chain complementarity determining region 2 (L-CDR2) comprising an amino acid sequence set forth in SEQ ID NO: 31; and
   f. a light chain complementarity determining region 3 (L-CDR3) comprising an amino acid sequence set forth in SEQ ID NO: 32.
Embodiment 14. The anti-human kappa light chain antibody or human kappa light chain binding fragment thereof of embodiment 13, further comprising:
   a. a heavy chain variable region comprising an amino acid sequence of SEQ ID NO: 25; and
   b. a light chain variable region comprising an amino acid sequence of SEQ ID NO: 29.
Embodiment 15. An anti-human kappa light chain antibody or human kappa light chain binding fragment thereof comprising:
   a. the heavy chain variable region comprising an amino acid sequence of SEQ ID NO: 25; and
   b. the light chain variable region comprising an amino acid sequence of SEQ ID NO: 29.
Embodiment 16. The anti-human kappa light chain antibody or human kappa light chain binding fragment thereof of embodiment 13, wherein:
   a. the heavy chain variable region comprises an amino acid sequence of SEQ ID NO: 25; or
   b. the light chain variable region comprises an amino acid sequence of SEQ ID NO: 29.
Embodiment 17. The anti-human kappa light chain antibody or human kappa light chain binding fragment thereof of any one of embodiments 1 to 4, wherein the anti-human κ light chain antibody isa human antibody.
Embodiment 18. The anti-human kappa light chain antibody or human kappa light chain binding fragment thereof of any one of embodiments 5-16, wherein the antibody is a humanized antibody.
Embodiment 19. The anti-human kappa light chain antibody or human kappa light chain binding fragment thereof of any one of embodiments 1 to 18, wherein the human κ light chain binding antibody fragment comprises a Fab, a Fab′, a F(ab′)2, a scFv, a dsFv, a ds-scFv, a dimer, a minibody, or a diabody.
Embodiment 20. The anti-human kappa light chain antibody or human kappa light chain binding fragment thereof of any one of embodiments 1 to 19, wherein the anti-human kappa light chain antibody or human kappa light chain binding fragment thereof is an IgG antibody.
Embodiment 21. The anti-human kappa light chain antibody or human kappa light chain binding fragment thereof of embodiment 20, wherein the IgG antibody is an IgG1 isotype.
Embodiment 22. The anti-human kappa light chain antibody or human kappa light chain binding fragment thereof of embodiment 20, wherein the IgG antibody is an IgG4 isotype.
Embodiment 23. The anti-human kappa light chain antibody or human kappa light chain binding fragment thereof of any one of embodiments 1 to 22, wherein the anti-kappa light chain antibody or human kappa light chain binding fragment thereof is coupled to a label.
Embodiment 24. The anti-human kappa light chain antibody or human kappa light chain binding fragment thereof of embodiment 23, wherein the label comprises a chemical label, a biological label, a fluorescent label, a radioisotope label, or any combination thereof.
Embodiment 25. The anti-human kappa light chain antibody or human kappa light chain binding fragment thereof of any one of embodiments 1 to 24, wherein the anti-human kappa light chain antibody or human kappa light chain binding fragment thereof binds to a kappa light chain aggregate.
Embodiment 26. The anti-human kappa light chain antibody or human kappa light chain binding fragment thereof of any one of embodiments 1 to 25, wherein the anti-human kappa light chain antibody or human kappa light chain binding fragment thereof is characterized by a melting temperature of at least about 70 °C, at least about 80 °C, or at least about 90 °C
Embodiment 27. The anti-human kappa light chain antibody or human kappa light chain binding fragment thereof of any one of embodiments 1 to 26, wherein the anti-human kappa light chain antibody or human kappa light chain binding fragment thereof is characterized by a higher binding affinity for a kappa light chain aggregate than a native kappa light chain.
Embodiment 28. The anti-human kappa light chain antibody or human kappa light chain binding fragment thereof of embodiment 1 to 27, wherein the anti-human kappa light chain antibody or human kappa light chain binding fragment thereof is characterized by an equilibrium constant (KD) for binding an kappa light chain aggregate that is no more than about 40 nM, no more than about 30 nM, no more than about 20 nM, no more than about 10 nM, no more than about 5 nM. no more than about 2 nM, no more than about 1 nM, no more than about 0.5 nM, no more than about 0.1 nM, no more than about 0.05 nM, or no more than about 0.01 nM.
Embodiment 29. The anti-human kappa light chain antibody or human kappa light chain binding fragment thereof of embodiment 1 to 27, wherein the anti-human kappa light chain antibody or human kappa light chain binding fragment thereof is characterized by an equilibrium constant (KD) for binding an kappa light chain aggregate that is at least about 0.01 nM, at least about 0.05 nM, at least about 0.1 nM, at least about 0.5 nM, at least about 1 nM, at least about 5 nM, or at least about 10 nM.
Embodiment 30. The anti-human kappa light chain antibody or human kappa light chain binding fragment thereof of embodiment 28 or embodiment 29, wherein the K_{D} is measured by an ELISA.
Embodiment 31. The anti-human kappa light chain antibody or human kappa light chain binding fragment thereof of embodiment 28 or embodiment 29, wherein the K_{D} is measured by a biolayer interferometry sensorgram.
Embodiment 32. The anti-human kappa light chain antibody or human kappa light chain binding fragment thereof of any one of embodiments 1 to 31, wherein the anti-human kappa light chain antibody or human kappa light chain binding fragment thereof is capable of being phagocytosed by a THP-1 monocyte cell line.
Embodiment 33. A pharmaceutical composition comprising the anti-human kappa light chain antibody or human kappa light chain binding fragment thereof of any one of embodiments 1 to 32 and a pharmaceutically acceptable excipient, diluent, or carrier.
Embodiment 34. The pharmaceutical composition of embodiment 33, wherein the pharmaceutical composition is formulated for intravenous administration.
Embodiment 35. The pharmaceutical composition of embodiment 33 or embodiment 34, for use in the treatment of a disease or condition characterized by kappa light chain aggregates in the subject.
Embodiment 36. The pharmaceutical composition of embodiment 35, wherein the disease or condition characterized by kappa light chain aggregates is a plasma cell disorder, systemic amyloidosis, or light chain amyloidosis.
Embodiment 37. The pharmaceutical composition of embodiment 35, wherein the plasma cell disorder is a plasma cell dyscrasia.
Embodiment 38. The pharmaceutical composition of embodiment 37, wherein the plasma cell dyscrasia is amyloidosis, monoclonal gammopathy of unknown significance (MGUS), multiple myeloma (MM), plasma cell leukemia (PCL), or any combination thereof.
Embodiment 39. The pharmaceutical composition of embodiment 37, wherein the plasma cell dyscrasia is systemic amyloidosis.
Embodiment 40. The pharmaceutical composition of embodiment 37, wherein the plasma cell dyscrasia is light chain amyloidosis.
Embodiment 41. The pharmaceutical composition of embodiment 37, wherein the plasma cell dyscrasia is a cardiac amyloidosis.
Embodiment 42. The pharmaceutical composition of embodiment 37, wherein the plasma cell dyscrasia is a renal amyloidosis.
Embodiment 43. A nucleic acid or a plurality of nucleic acids encoding the anti-human kappa light chain antibody or human kappa light chain binding fragment thereof of any one of embodiments 1 to 32.
Embodiment 44. A cell comprising a nucleic acid or a plurality of nucleic acids encoding the anti-human kappa light chain antibody or human kappa light chain binding fragment thereof of any one of embodiments 1 to 32.
Embodiment 45. The cell of embodiment 44, wherein the cell is a eukaryotic cell.
Embodiment 46. The cell of embodiment 45, wherein the eukaryotic cell is a Chines Hamster Ovary (CHO) cell.
Embodiment 47. The cell of embodiment 44, wherein the cell is a prokaryotic cell.
Embodiment 48. A method of diagnosing amyloidosis in a sample, the method comprising contacting a sample with the anti-human kappa light chain antibody or human kappa light chain binding fragment of any one of embodiments 1 to 32.
Embodiment 49. A method of detecting a kappa light chain aggregate in a sample, the method comprising contacting the sample with the anti-human kappa light chain antibody or human kappa light chain binding fragment thereof of any one of embodiments 1 to 32.
Embodiment 50. The method of any one of embodiments 48 to 49, wherein the sample comprises blood, serum, plasma, solid tissue, liquid biopsy, bone marrow, cerebrospinal fluid, or any combination thereof.
Embodiment 51. The method of embodiment 49 or embodiment 50, wherein the sample comprises a cardiac tissue, a kidney tissue, a pulmonary tissue, an intestinal tissue, a gastrointestinal (GI) tissue, a skin tissue, a subcutis tissue, a soft tissue, a tendon tissue, a bone tissue, a synovial tissue, a gall bladder tissue, a pharyngeal tissue, a spleen tissue, a lymph node tissue, a neural tissue, or an immune tissue.
Embodiment 52. The method of embodiment 49, wherein the sample comprises a cardiac tissue.
Embodiment 53. The method of embodiment 49, wherein the sample comprises a renal tissue.
Embodiment 54. The method of any one of embodiments 48 to 51, wherein the sample is obtained from a subject.
Embodiment 55. The method of embodiment 54, wherein the subject is a human.
Embodiment 56. A method of increasing clearance of a kappa light chain aggregate, the method comprising contacting the kappa light chain aggregate with the anti-human kappa light chain antibody or human kappa light chain binding fragment thereof of any one of embodiments 1 to 32.
Embodiment 57. The method of embodiment 56, wherein the method results in a decrease in the level of the kappa light chain aggregate in a cardiac tissue, a renal tissue, a pulmonary tissue, an intestinal tissue, a gastrointestinal (GI) tissue, a soft tissue, a synovial tissue, a spleen tissue, or an immune tissue compared to a level of the kappa light chain aggregate in a similar tissue prior to the contacting.
Embodiment 58. The method of embodiment 56, wherein the method results in a decrease in the level of the kappa light chain aggregate in a cardiac tissue compared to a level of the kappa light chain aggregate in a similar cardiac tissue prior to the contacting.
Embodiment 59. The method of embodiment 56, wherein the method results in a decrease in the level of the kappa light chain aggregate in a similar renal tissue compared to a level of the kappa light chain aggregate in a similar renal tissue prior to the contacting.
Embodiment 60. The method of embodiment 56, wherein the method results in a decrease in the level of the kappa light chain aggregate in a colon tissue compared to a level of the kappa light chain aggregate in a colon tissue prior to the contacting. A method of reducing kappa light chain aggregates in a subject in need thereof, the method comprising administering the anti-human kappa light chain antibody or human kappa light chain binding fragment thereof of any one of embodiments 1 to 32 to the subject, thereby reducing a level or the number of kappa light chain aggregates in the subject.
Embodiment 61. The method of embodiment 60, wherein the method results in a decrease in the level or the number of the kappa light chain aggregate in a cardiac tissue, a renal tissue, a pulmonary tissue, an intestinal tissue, a gastrointestinal (GI) tissue, a soft tissue, a synovial tissue, a spleen tissue, or an immune tissue compared to a level or a number or a number of the kappa light chain aggregate in a similar tissue prior to the administering.
Embodiment 62. The method of embodiment 60, wherein the method results in a decrease in the level or the number of the kappa light chain aggregate in a cardiac tissue compared to a level or a number of the kappa light chain aggregate in a similar cardiac tissue prior to the administering.
Embodiment 63. The method of embodiment 60, wherein the method results in a decrease in the level or the number of the kappa light chain aggregate in a renal tissue compared to a level or a number of the kappa light chain aggregate in a similar renal tissue prior to the administering.
Embodiment 64. The method of embodiment 60, wherein the method results in a decrease in the level or the number of the kappa light chain aggregate in a similar colon tissue compared to a level or a number of the kappa light chain aggregate in a similar colon tissue prior to the administering.
Embodiment 65. A method of treating a disease or condition characterized by kappa light chain aggregates in a subject in need thereof, the method comprising administering the anti-human kappa light chain antibody or human kappa light chain binding fragment thereof of any one of embodiments 1 to 32 to the subject, thereby treating the disease or condition characterized by kappa light chain aggregates in the subject.
Embodiment 66. The method of embodiment 65, wherein the disease or condition characterized by kappa light chain aggregates is a plasma cell disorder.
Embodiment 67. The method of embodiment 66, wherein the plasma cell disorder comprises a plasma cell dyscrasias.
Embodiment 68. The method of embodiment 67, wherein the plasma cell dyscrasias comprises an amyloidosis, a monoclonal gammopathy of unknown significance (MGUS), multiple myeloma (MM), plasma cell leukemia (PCL), or any combination thereof.
Embodiment 69. The method of embodiment 65, wherein the disease or condition characterized by kappa light chain aggregates is systemic amyloidosis.
Embodiment 70. The method of embodiment 65, wherein the disease or condition characterized by kappa light chain aggregates is light chain amyloidosis.
Embodiment 71. The method of embodiment 68, wherein the disease or condition characterized by kappa light chain aggregates is a cardiac amyloidosis.
Embodiment 72. The method of embodiment 68, wherein the disease or condition characterized by kappa light chain aggregates is a renal amyloidosis.
Embodiment 73. The method of embodiment 71 or embodiment 72, wherein the administering the anti-human kappa light chain antibody or human kappa light chain binding fragment thereof of any one of embodiments 1 to 32 to the subject results in a reduction of kappa light chain aggregates in the subject.
Embodiment 74. The method of embodiment 60 or 65, wherein administering the anti-human kappa light chain antibody or human kappa light chain binding fragment thereof of any one of embodiments 1 to 32 to the subject increases a number of macrophages or monocytes surrounding or within an amyloidoma in the subject.
Embodiment 75. The method of embodiment 60 or 65, wherein the administering the anti-human kappa light chain antibody or human kappa light chain binding fragment thereof increases a number of macrophages surrounding or within an amyloidoma in the subject.
Embodiment 76. The method of embodiment 74 or 75, wherein the macrophage is a F4/80+ macrophage.
Embodiment 77. The method of embodiment 60 or 65, wherein the administering the anti-human kappa light chain antibody or human kappa light chain binding fragment thereof increases phagocytic uptake of kappa light chain aggregates.
Embodiment 78. The method of embodiment 60 or 65, wherein the administering the anti-human kappa light chain antibody or human kappa light chain binding fragment thereof decreases a size of an amyloidoma in the subject.
Embodiment 79. The method of embodiment 78, wherein the method results in a decrease in the size of the kappa light chain aggregate in a cardiac tissue, a renal tissue, a pulmonary tissue, an intestinal tissue, a gastrointestinal (GI) tissue, a soft tissue, a synovial tissue, a spleen tissue, or an immune tissue compared to a size of the kappa light chain aggregate in a similar tissue prior to the administering.
Embodiment 80. The method of embodiment 78, wherein the method results in a decrease in the size of the kappa light chain aggregate in a cardiac tissue compared to a size of the kappa light chain aggregate in a similar cardiac tissue prior to the administering.
Embodiment 81. The method of embodiment 78, wherein the method results in a decrease in the size of the kappa light chain aggregate in a renal tissue compared to a size of the kappa light chain aggregate in a similar renal tissue prior to the administering.
Embodiment 82. The method of embodiment 78, wherein the method results in a decrease in the size of the kappa light chain aggregate in a similar colon tissue compared to a size of the kappa light chain aggregate in a similar colon tissue prior to the administering.
Embodiment 83. A method of manufacturing the anti-human kappa light chain antibody or human kappa light chain binding fragment thereof of any one of embodiments 1 to 32, the method comprising culturing a cell comprising a nucleic acid encoding the anti-human kappa light chain antibody or human kappa light chain binding fragment thereof under conditions sufficient to allow production and secretion of the anti-human kappa light chain antibody or the human kappa light chain binding fragment thereof from the cell.
Embodiment 84. The method of embodiment 83, further comprising isolating the anti-human kappa light chain antibody or the human kappa light chain binding fragment thereof from the cell or a supernatant of the cell.
Embodiment 85. The method of embodiment 83 or 84, wherein the cell is a eukaryotic cell.
Embodiment 86. The method of any one of embodiments 61-85, wherein the eukaryotic cell comprises a Chinese Hamster Ovary (CHO) cell.
Embodiment 87. An anti-human kappa light chain antibody or human kappa light chain binding fragment thereof produced by a method of any one of embodiments 83-86.
Embodiment 88. A vector comprising:
   a) a first nucleic acid sequence encoding a heavy chain variable region comprising an amino acid sequence as set forth in SEQ ID NO: 1, or encoding a heavy chain variable region comprising an H-CDR1, a H-CDR2, and a H-CDR3 as set forth in the amino acid sequence of SEQ ID NO: 1, and/or a second nucleic acid encoding a light chain variable region comprising an amino acid sequence as set forth in SEQ ID NO: 5, or encoding a light chain variable region comprising an L-CDR1, an L-CDR2, and an L-CDR3 as set forth in the amino acid sequence of SEQ ID NO: 5;
   b) a first nucleic acid sequence encoding a heavy chain variable region comprising an amino acid sequence as set forth in SEQ ID NO: 9, or encoding a heavy chain variable region comprising an H-CDR1, a H-CDR2, and a H-CDR3 as set forth in the amino acid sequence of SEQ ID NO: 9, and/or a second nucleic acid encoding a light chain variable region comprising an amino acid sequence as set forth in SEQ ID NO: 13, or encoding a light chain variable region comprising an L-CDR1, an L-CDR2, and an L-CDR3 as set forth in the amino acid sequence of SEQ ID NO: 13;
   c) a first nucleic acid sequence encoding a heavy chain variable region comprising an amino acid sequence as set forth in SEQ ID NO: 17, or encoding a heavy chain variable region comprising an H-CDR1, a H-CDR2, and a H-CDR3 as set forth in the amino acid sequence of SEQ ID NO: 17, and/or a second nucleic acid encoding a light chain variable region comprising an amino acid sequence as set forth in SEQ ID NO: 21, or encoding a light chain variable region comprising an L-CDR1, an L-CDR2, and an L-CDR3 as set forth in the amino acid sequence of SEQ ID NO: 21; or
   d) a first nucleic acid sequence encoding a heavy chain variable region comprising an amino acid sequence as set forth in SEQ ID NO: 25, or encoding a heavy chain variable region comprising an H-CDR1, a H-CDR2, and a H-CDR3 as set forth in the amino acid sequence of SEQ ID NO: 25, and/or a second nucleic acid encoding a light chain variable region comprising an amino acid sequence as set forth in SEQ ID NO: 29, or encoding a light chain variable region comprising an L-CDR1, an L-CDR2, and an L-CDR3 as set forth in the amino acid sequence of SEQ ID NO: 29.
Embodiment 89. Use of the anti-human kappa light chain antibody or human kappa light chain binding fragment thereof of any one of embodiments 1 to 32 in the manufacture of a medicament for the treatment of a disease or condition characterized by kappa light chain aggregates in the subject.
Embodiment 90. The use of embodiment 89, wherein the disease or condition characterized by kappa light chain aggregates is a plasma cell disorder, systemic amyloidosis, or light chain amyloidosis.
Embodiment 91. The use of embodiment 90, wherein the plasma cell disorder is a plasma cell dyscrasia.
Embodiment 92. The use of embodiment 91, wherein the plasma cell dyscrasia is amyloidosis, monoclonal gammopathy of unknown significance (MGUS), multiple myeloma (MM), plasma cell leukemia (PCL), or any combination thereof.
Embodiment 93. The use of embodiment 90, wherein the amyloidosis is a systemic amyloidosis.
Embodiment 94. The use of embodiment 90, wherein the amyloidosis is a light chain amyloidosis.
Embodiment 95. The use of embodiment 90, wherein the amyloidosis is a cardiac amyloidosis.
Embodiment 96. The use of embodiment 90, wherein the amyloidosis is a renal amyloidosis.

### EXAMPLES

The following illustrative examples are representative of embodiments of compositions and methods described herein and are not meant to be limiting in any way.

### Example 1—Properties of Anti-Human κ Light Chain Antibodies

The problem solved, at least in part, was to create conformation-specific antibodies that bind the toxic κ light chain deposited in organs or their misfolded precursors in circulation, while leaving the well-folded, circulating κ light chains untouched. Solving this problem provides a solution to directly addressing the disease-causing species with high potency while avoiding interactions with the often-abundant, non-pathological folded, free light chains that could otherwise function as a biological sink during therapy or lead to unwanted immune side effects. Without being bound by theory binding of the antibody to the misfolded species targets them for removal via an antibody-dependent cellular phagocytosis (ADCP) mechanism.

Light chains are ~214 amino acid proteins consisting of a variable domain (V_{L}, amino acids (AA) 1-108) and a constant domain (CL, AA 109-214). The variable domain contains hypervariable regions which consists of different amino acid sequences in every patient, whereas the constant domain shares sequence identity in all patients with the same isotype (x or k). It is thought that in each patient, their unique light chain's variable domain sequence results in the protein's unique properties such as amyloidogenic propensity or amyloid structure, and much of the research efforts have been made to target this domain.

The anti-human κ light chain antibodies described herein were generated to bind a misfolding-specific epitope, STYSLSSTLT (SEQ ID NO: 33), on the κ light chain protein. Four clones were identified and purified from mouse ascites fluid for subsequent screening of desirable misfolding-specific properties.

### Thermal Stability of κ Light Chain Antibodies

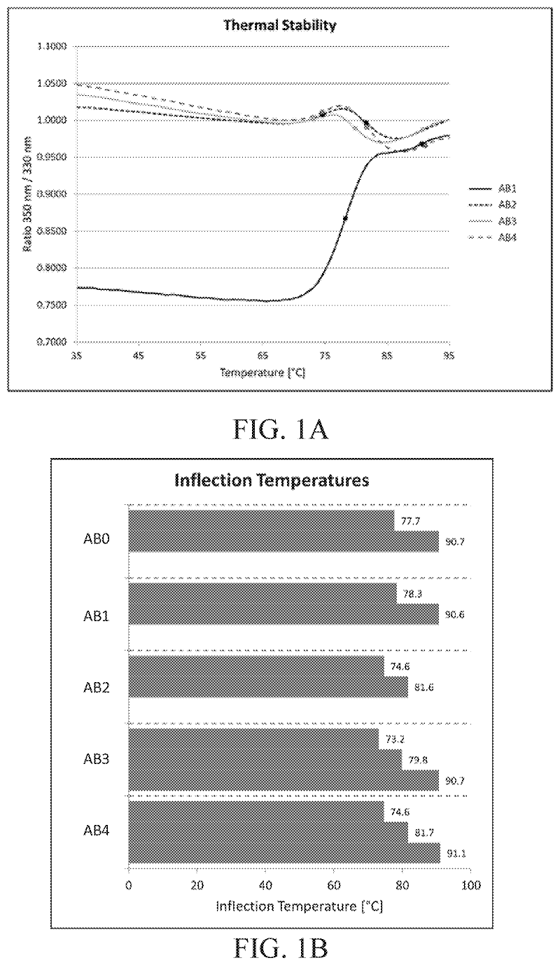

Initially, the thermal stability of four monoclonal anti-human κ light chain antibodies of this disclosure was assessed in samples of purified antibody at a concentration of 1 mg/mL. The ratio of fluorescence at 350 nm/330 nm was calculated based upon inflection temperatures measured using a Nanotemper Tycho™. Inflection points for each of antibody are indicated in the melting curves shown in FIG. 1A and represent the melting temperature (Tₘ), which are quantified in FIG. 1B. Inflection/melting temperatures: AB1 (78.3 °C, 90.6 °C), AB2 (74.6 °C, 81.6 °C), AB3 (73.2 °C, 79.8 °C, 90.7 °C), and AB4 (74.6 °C, 81.7 °C, 91.1 °C).

### Preferential Binding to Misfolded Versus Native Conformations of κ Light Chains

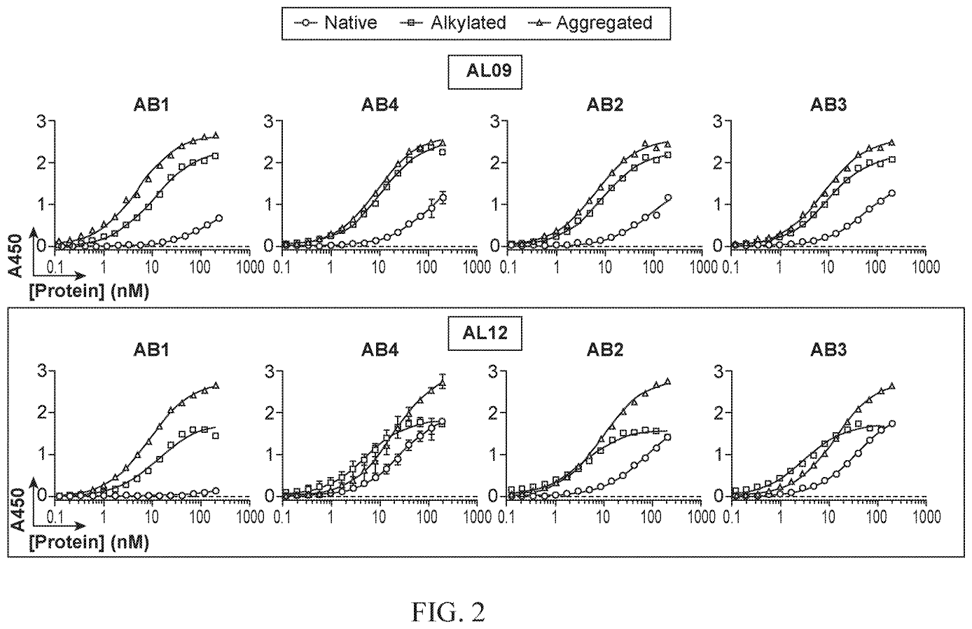

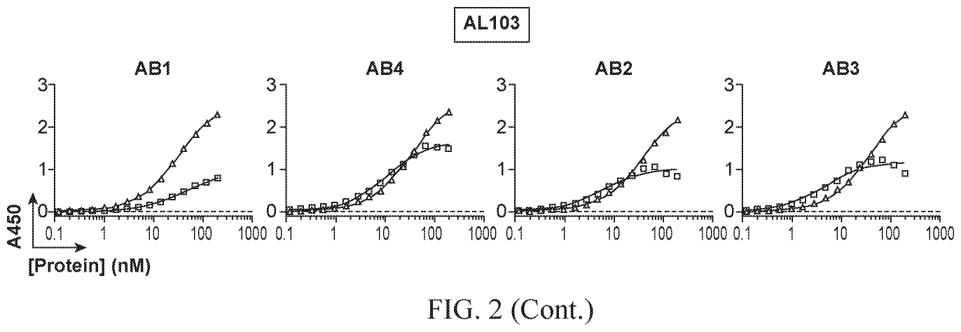

The ability of four clones to bind misfolded κ light chains was then tested using capture ELISA to determine whether the four antibodies can recognize and preferentially bind to misfolded kappa light chain variants sequences derived from 3 different amyloidosis patients: AL09, AL12, and AL103. The monoclonal antibodies (20 ng) were captured upright on-plate with anti-IgG Fc antibodies. Biotinylated kappa light chain variants were added to each monoclonal antibody at varying concentrations for 1 hour. Neutravidin-HRP was added at a dilution factor of 1:20,000 to each reaction for 30 mins. Colour was developed with TMB substrate for 150-180 sec. Binding curves are shown in FIG. 2 and were fitted using a Michaelis-Menten model of GraphPad Prism v10.2.3 software to quantify the equilibrium constant (K_{D}, nM). TABLE 2 lists the K_{D} values for each antibody binding to native, alkylated, or aggregated κ light chains. These data demonstrate that each of the tested monoclonal antibodies generally have a stronger binding affinity to alkylated and aggregated κ light chains compared to the native conformation. Notably, the AB1 antibody exhibited the highest distinction with respect to binding specificity to alkylated and aggregated κ light chains compared to the native forms.

TABLE 2
Equilibrium constants (K_{D}, nM) of human and humanized
anti-κ light chain monoclonal antibodies
to AL09, AL12, and AL103 κ light chain variants

| | AL09 | | | AL12 | | | AL103 | |
|---|---|---|---|---|---|---|---|---|
| Antibody | Native | Alk | Agg | Native | Alk | Agg | Alk | Agg |
| AB1 | 184 | 11 | 4.8 | 335 | 12.6 | 8.64 | 41.3 | 32.8 |
| AB4 | 101 | 10.8 | 8.95 | 24.4 | 6.82 | 12 | 11.3 | 37.6 |
| AB2 | 91 | 9 | 6.4 | 60.3 | 3.38 | 9.04 | 6.13 | 43 |
| AB3 | 61 | 9.4 | 8 | 37.7 | 3.09 | 13.3 | 4.61 | 39.6 |

A competition ELISA was then performed on the AB1, AB4, AB2, and AB3 clones to determine if natively folded κ light chains could compete for binding to κ light chains aggregates obtained from patient sample AL09. Monoclonal antibodies (20 ng) were captured upright on-plate with anti-IgG Fc antibodies. On a separate plate, the biotinylated analyte (2 nM aggregated AL09) was mixed with serially diluted competitor (AL09 native κ light chains or pooled Igκ light chains). The mixed solution was added to candidate mAb plate and incubated for 2 hours at room temperature. Neutravidin-HRP was added at 1:15,000 dilution for 30 min. Colour was developed with TMB substrate for 210 sec. Competition binding curves for ABI, AB4, AB2, and AB3 antibodies demonstrated that all 4 tested antibodies exhibited high conformation specificity to misfolded κ light chains and a low binding affinity to native κ light chains or pooled Ig κ light chains (FIG. 3A, FIG. 3B, FIG. 3C, and FIG. 3D). Natively folded pooled kappa light chains did not compete for binding to AL09 aggregates (2 nM) even at 1000-fold higher concentration of competitor. The Y-axis of each competition binding curve is normalized to reaction wells without competitor, which represents 100% analyte still bound. TABLE 3A shows IC₅₀ in nanomolar (nM) concentration obtained from Graphpad Prism v10.2.3 software using [Inhibitor]vs. Response—(three parameters) curve fit. Unstable or inaccurate fits are stated as >2000 nM.

TABLE 3A
Antibody IC₅₀ (nM) of human and humanized anti-κ light chain
monoclonal antibodies to AL09 κ light chain variant and
conformations assessed by competition ELISA
IC₅₀ (nM)

| | AL09 κ light chain variant | | | Pooled |
|---|---|---|---|---|
| Antibody | Native | Alkylated | Aggregated | Igκ LC |
| AB1 | >2000 | 316 | 441 | >2000 |
| AB4 | >2000 | 90.5 | 298 | >2000 |
| AB2 | >2000 | 52 | 378.5 | >2000 |
| AB3 | 1752 | 40.33 | 238.7 | >2000 |

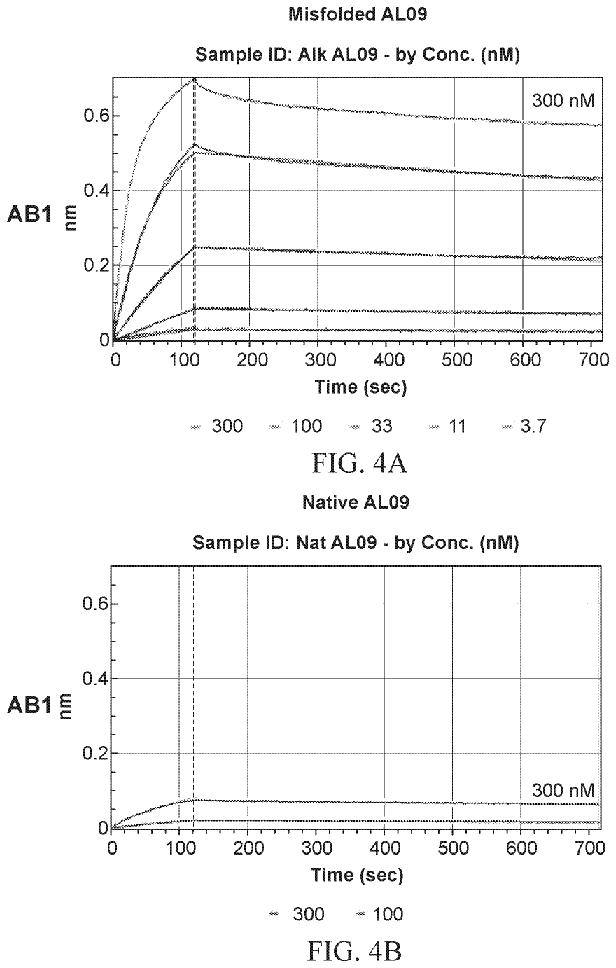

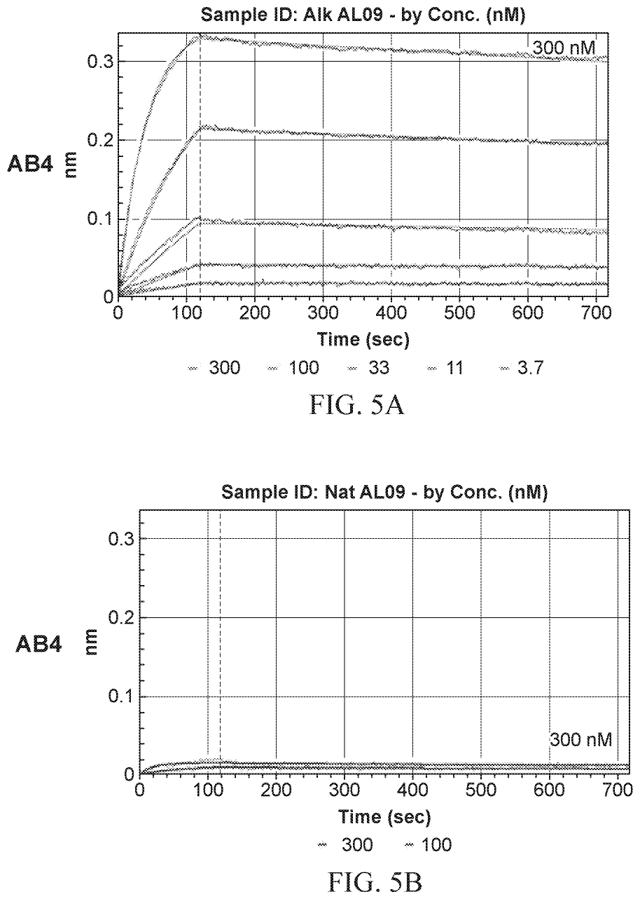

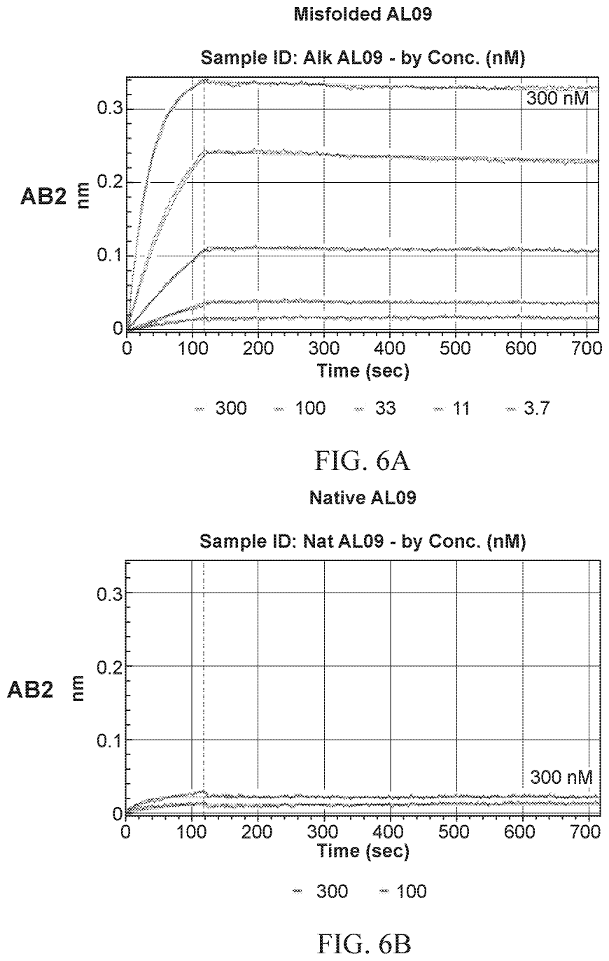

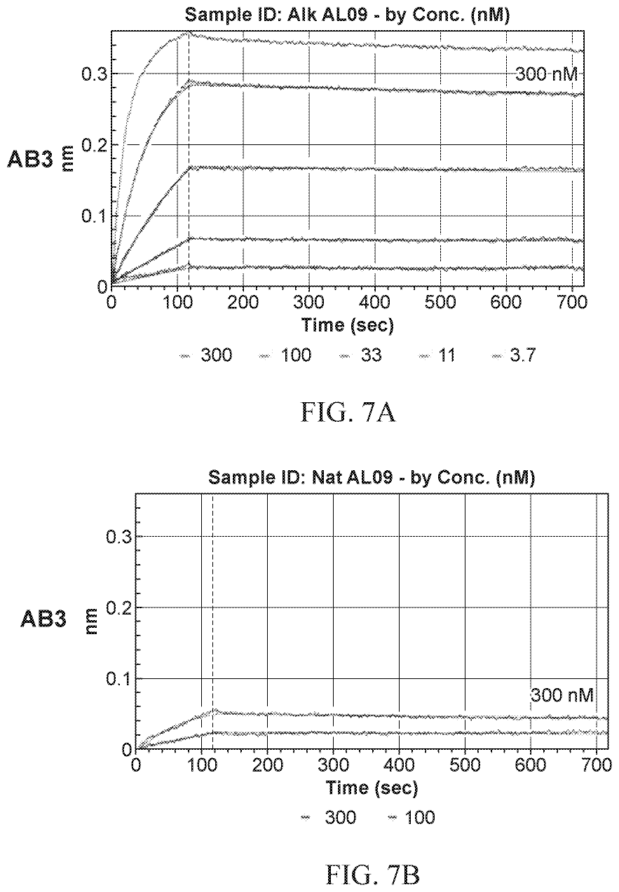

Biolayer interferometry (BLI) sensorgrams were also performed to determine the binding of each of the 4 monoclonal antibodies to misfolded κ light chain variants. Binding to alkylated κ light chains AL09 variants (presented at 300, 100, 33, 11, and 3.7 nM) and to native AL09 κ light chains at concentrations of 300 nM and 100 nM were measured for AB1 (FIG. 4A, FIG. 4B), AB4 (FIG. 5A, FIG. 5B), AB2 (FIG. 6A, FIG. 6B), and AB3 (FIG. 7A, FIG. 7B) antibodies. The association time and dissociation time for all reactions were 120 seconds and 600 seconds, respectively. We performed similar experiments in kappa light chain samples obtained from myeloma patients using BLI sensorgrams, which demonstrated that neither the AB2 nor the AB4 antibody was able to bind native pooled light chains (data not shown).

The dissociation constant (K_{D}, nM) of each antibody was calculated by curve fitting with GraphPad Prism and provided in TABLE 3B. The data demonstrated that each of the 4 tested antibodies exhibited at least about an 8-fold increase in binding to 300 nM of misfolded AL09 K light chains compared to 300 nM of native AL09 K light chains. Moreover, about a 10-fold to 20-fold increase in binding to 100 nM alkylated AL09 K light chains was observed for each of the 4 clones compared to 100 nM native AL09 K light chains. Taken together, these data corroborated earlier competition ELISA results and demonstrated that the AB1, AB4, AB2, and AB3 clones bind strongly to misfolded κ light chains while negligibly binding to native κ forms.

TABLE 3B
Binding affinities (KD, nM) of human and humanized anti-
κ light chain monoclonal antibodies to alkylated
AL09 κ light chain variant from data in FIGs. 4A-7B

| Antibody | K_{D}, alkylated AL09 (nM) |
|---|---|
| AB1 | 1.73 ± 0.01 |
| AB4 | 1.89 ± 0.01 |
| AB2 | 0.67 ± 0.01 |
| AB3 | 0.42 ± 0.01 |

### Improved Specificity and Binding of Constant Region-Targeting Anti-KLC Antibodies to Misfolded KLCs Compared to Variable Region-Targeting Anti-KLC Antibodies

We also compared the binding and specificity of the constant region-targeting abilities AB1, AB2, AB3, and AB4 described herein against two other anti-kappa light chain antibodies that target the variable region of the kappa light chains: vrAB1 and vrAB2 (clone 11-1F4 in WO2022056484A1 and clone 2A4 in WO2013063284A1).

We first extended our competition ELISA to directly compare AB1, AB2, and AB4 anti-KLC antibodies with the vrAB1 and vrAB1 antibodies in the presences of native AL09, alkylated AL09, aggregated AL09, or pooled KLCs. All of the tested AB1, AB2, and AB4 anti-KLC antibodies exhibited significantly improved binding to both alkylated and aggregated KLC competitors than both vrAB1 and vrAB2, whereby the natively folded and pooled kappa light chains did not compete for binding to AL09 aggregates (2 nM) even at 1000-fold higher concentration of vrAB1 and vrAB2 (FIGS. 3E-3I). The IC₅₀ of AB2 and AB4 for binding to misfolded κLCs were calculated to be 60.84 nM and 62.36 nM, respectively, based on the competition curves in FIG. 3F and FIG. 3G. The IC₅₀ of AB2 and AB4 for binding to aggregated κLCs were calculated to be 927.2 nM and 968.8 nM, respectively.

Another antibody capture ELISA was then performed using the same conditions described above to compare the affinities (K_{D}) of our anti-K light chain antibodies AB1, AB2, and AB4 to both vrAB1 and vrAB2 antibodies for binding misfolded light chains. Here, we measured the binding of these antibodies to misfolded κLCs and κLC fibrils from AL09, AL12, AL103 samples (patients with disease-causing light chains sequences utilizing IGKV1 germline segments), as well as misfolded kappa light chains obtained from patients with light chain sequences utilizing IGKV4 germline segments (e.g., LEN and SMA). These data demonstrate that all of AB1, AB2, and AB4 have superior affinity to misfolded κLCs in all patient samples tested than vrAB2 (FIGS. 3J, 3K, 3L, 3N). (FIGS. 3J, 3K, 3L, 3M). Moreover, the AB2 and AB4 antibodies proved to have K_{D} values that were ~5-10-fold less than vrAB1 for binding the IGKV1 misfolded κLCs, as shown in TABLE 3C.

TABLE 3C
Binding affinity (K_{D}) of antibodies to misfolded κLC in solid phase

| K_{D} (nM) | |
|---|---|
| κLC | |
| IGKV1 | IGKV4 |

| Antibody | AL09 | AL12 | AL103 | LEN | SMA |
|---|---|---|---|---|---|
| AB1 | 11 | 15 | 25.4 | 6.5 | 4.35 |
| AB4 | 10.4 | 3.72 | 4.03 | 4.73 | 3.01 |
| AB2 | 9.51 | 2.85 | 7.32 | 4.51 | 3.39 |
| vrAB1 | 11.9 | 20.9 | 39.4 | 1.57 | 0.98 |
| vrAB2 | 43.6 | n.b. | n.b. | n.b. | n.b. |

n.b. = no binding detected

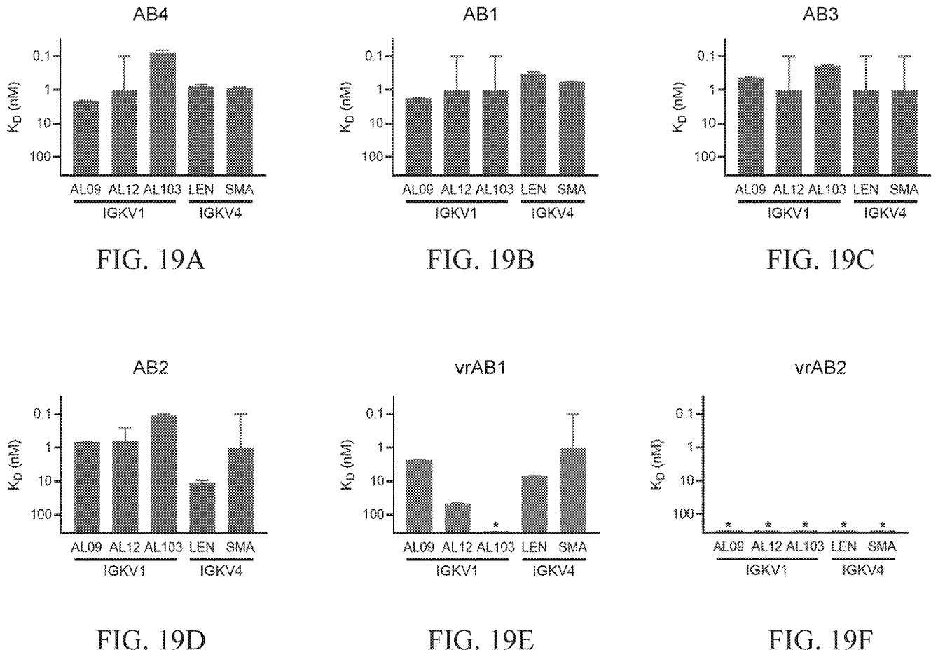

BLI sensorgrams were also used to measure and compare the binding affinities of AB1, AB2, AB3, and AB4 as well as vrAB1 and vrAB2 to misfolded κLCs and fibrils. Misfolded κLCs and fibrils from AL09, AL12, AL103 samples (patient light chain sequences utilizing IGKV1 germline segments), as well as misfolded kappa light chains obtained from patients sequences utilizing IGKV4 germline segments (e.g., LEN and SMA), were mixed with ABI, AB2, AB3, and AB4 antibodies or with variable region-targeting anti-κLC antibodies reference antibodies: vrAB1 and vrAB2. As shown in TABLE 3D and FIGS. 19A-19F, all of the ABI, AB2, AB3, and AB4 antibodies exhibited reliable high binding affinity to misfolded κ light chains, regardless of κLC variant sequence or v-gene usage of the tested sample. Compared to the variable region-targeting antibodies vrAB1 and vrAB2, the measured K_{D} values of AB1, AB2, AB3, and AB4 antibodies were significantly lower for nearly all samples tested. Notably, vrAB2 exhibited essentially no binding to any of the tested samples with K_{D} values measured consistently >300 nM. The binding affinity of vrAB1 had highly variable binding affinity to misfolded κLCs of all tested samples, ranging from no measured binding to AL103 misfolded κLCs to as high as −45 nM binding to AL12 misfolded KLCs. ABI, AB2, AB3, and AB4 constant region-targeting anti-kappa light chains described herein had lower K_{D} values than vrAB1 for binding misfolded κLCs in all IGKV1 variant samples, and the K_{D} values of AB2 and AB3 were ~70% and ~80% lower than that of vrAB1. Only for the IGKV4 variants were the binding affinities comparable between AB2 and vrAB1.

TABLE 3D
Binding affinities of constant region-targeting and variable region-targeting anti-κLC antibodies

| Targeted κLC | mAb | IGKV1 variants | | | IGKV4 variants | |
|---|---|---|---|---|---|---|
| epitope region | ID | AL09 | AL12 | AL103 | LEN | SMA |
| constant κLC | AB1 | 1.73 ± 0.01 | positive* | positive* | 0.32 ± 0.03 | 0.56 ± 0.01 |
| region | AB4 | 2.09 ± 0.03 | positive* | 0.076 ± 0.010 | 0.76 ± 0.07 | 0.85 ± 0.02 |
| | AB2 | 0.67 ± 0.01 | 0.63 ± 0.38 | 0.11 ± 0.01 | 10.8 ± 1.6 | positive* |
| | AB3 | 0.42 ± 0.01 | 0.010 ± 0.004 | 0.19 ± 0.01 | positive* | positive* |
| variable κLC | vrAB1 | 2.31 ± 0.04 | 45 ± 1 | none | 7.0 ± 0.2 | positive* |
| region | | | | (>300)^{†} | | |
| | vrAB2 | none | none | none | none | none |
| | | (>1000)^{†} | (>1000)^{†} | (>300)^{†} | (>300)^{†} | (>1000)^{†} |

*indicates off-rate too slow to fit, or possible accumulation of antigen on sensor tip.
^{^{†}}none (>x) indicates no fittable binding at a maximum tested antigen concentration of x nM.

We also measured and compared the avidities of the AB1, AB2, and AB4 with vrAB1 and vrAB2 antibodies to 20 μM adsorbed and dried AL09 misfolded κLCs using an indirect ELISA. This experiment revealed that all three of ABI, AB2, and AB4 antibodies had binding capacities that were at least ~2-fold higher than the vrAB1 antibody (FIG. 3O), and the binding capacity of the AB2 antibody was ~5-fold higher than vrAB1. The vrAB2 was determined to have minimal binding capacity for the adsorbed and dried AL09 misfolded κLCs. These data suggest that our constant region-targeting anti-κLC antibodies could bind to misfolded κLCs and κLC fibrils with higher binding and specificity than the variable region-targeting vrAB1 and vrAB2 variable region-targeting antibodies.

### Example 2—Anti-Human κ Light Chain Antibodies are Efficiently Phagocytosed by Immune Cells

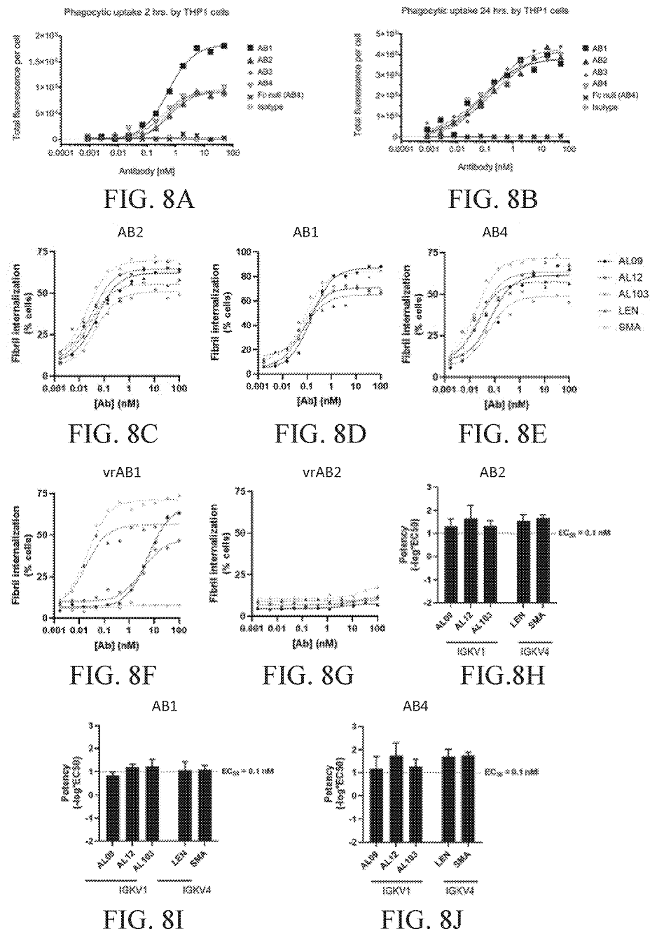

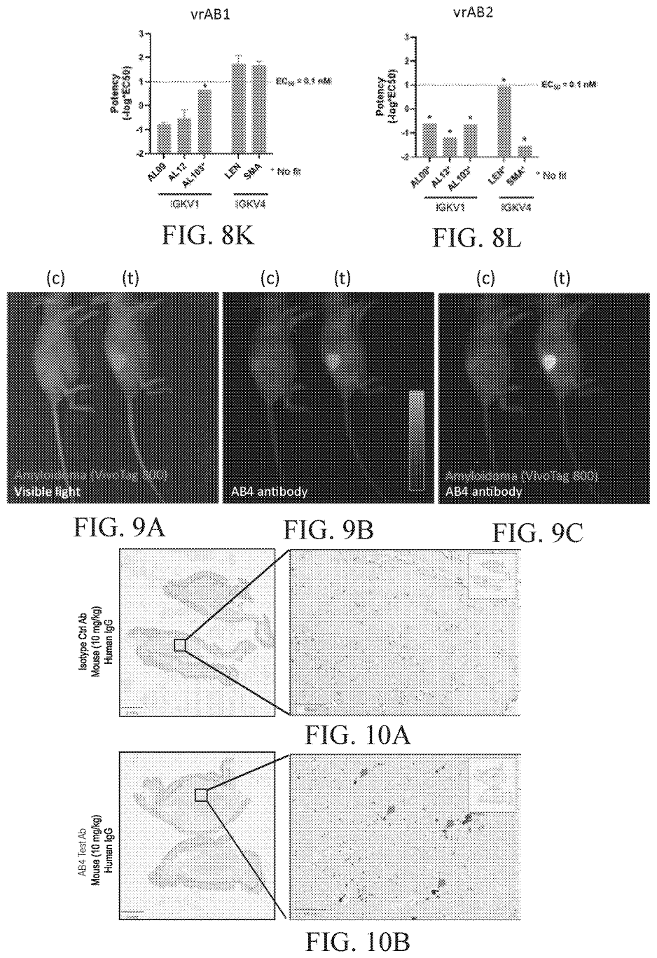

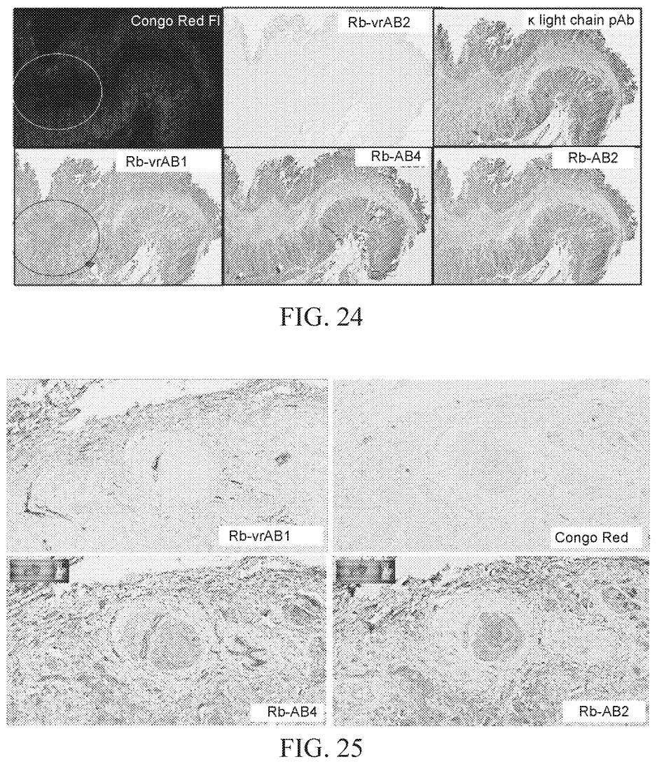

We performed a phagocytosis assay using a monocytic THP-1 cell line to determine if the candidate antibodies could be effectively delivered intracellularly to cells containing misfolded κ light chain aggregates. THP-1 cells were pre-incubated in a mixture of pHrodo-labelled κ light chain aggregates (160 nM) and increasing concentrations of either AB1, AB4, AB2, or AB3 for 2 hours (FIG. 8A) or 24 hours (FIG. 8B). Fluorescence imaging was performed using a Cytation™ 5 microscopy system (Agilent Systems), and the total fluorescence per cell in each well was plotted. The EC₅₀ for each antibody was calculated by curve fitting the data in FIG. 8A and FIG. 8B and shown in TABLE 4. Each of the 4 tested antibodies exhibited sub-nanomolar EC₅₀ values after the 24-hour incubation, and AB1 displayed the fastest uptake kinetics (i.e. rate of phagocytosis) compared to the AB4, AB2, and AB3 clones.

TABLE 4
Half maximal effective concentration (EC50) of cellular uptake
kinetics of human and humanized anti-κ light chain
monoclonal antibodies in THP-1 cells
EC₅₀ (nM)

| | | | | | Fc null |
|---|---|---|---|---|---|
| Time | AB1 | AB2 | AB3 | AB4 | (AB4) |
| 2 hours | 0.541 | 0.627 | 0.498 | 0.376 | N/A |
| 24 hours | 0.0737 | 0.221 | 0.134 | 0.0789 | N/A |

We then extended the phagocytosis assay to measure uptake AL09, AL12, AL103, LEN, and SMA misfolded κLCs in THP1 cells by each the candidate anti-κ light chain antibodies. The results demonstrated that ABI, AB2, and AB4 increased uptake of misfolded κLCs from all IGKV1 and IGKV4 patient samples in at least ~50-85% of THP1 cells (FIGS. 8C-8E). In contrast, the variable region-targeting vrAB1 antibody required much higher concentrations for increasing uptake to κLCs of the IGKV1 samples, AL09 and AL12, and did not induce uptake of AL103 κLCs in THP-1 cells at any of the tested concentrations (FIG. 8F). The vrAB2 antibody was unable to cause uptake of any κLC samples tested (FIG. 8G). The average potency (EC₅₀) of phagocytic uptake were calculated for each antibody (FIGS. 8H-8L) and were based on the concentration curves shown FIGS. 8C-8G.

### Example 3—Anti-Human κ Light Chain Antibodies are Recruited to Amyloidoma

We next performed fluorescent imaging experiments to test whether the antibody candidates could co-localize to kappa amyloid aggregates in vivo. Here, control mice were subcutaneously injected with Matrigel only, while test mice received a subcutaneous injection of 0.5 mg of VivoTag-800-conjugated κ light chain amyloid protein and Matrigel to create an amyloidoma in vivo. All mice were then administered a 10 mg/kg dose of an Alexa-680 dye-conjugated AB4 monoclonal antibody via a tail vein injection. Fluorescent images were then acquired of both control and test mice after 4 days following intravenous administration of the Alexa-680 AB4 antibody to visualize and quantify the colocalization of the antibody and the amyloidoma in vivo (FIG. 9A, FIG. 9B, and FIG. 9C). These data demonstrate that the AB4 antibody targets amyloidomas in vivo with high specificity and negligible off-target binding in other tissues. Given the comparable K_{D} values and preferential binding to misfolded κ light chain proteins, we further hypothesize that the AB1, AB2, and AB3 would similarly target and colocalize to amyloidomas in vivo following intravenous administration.

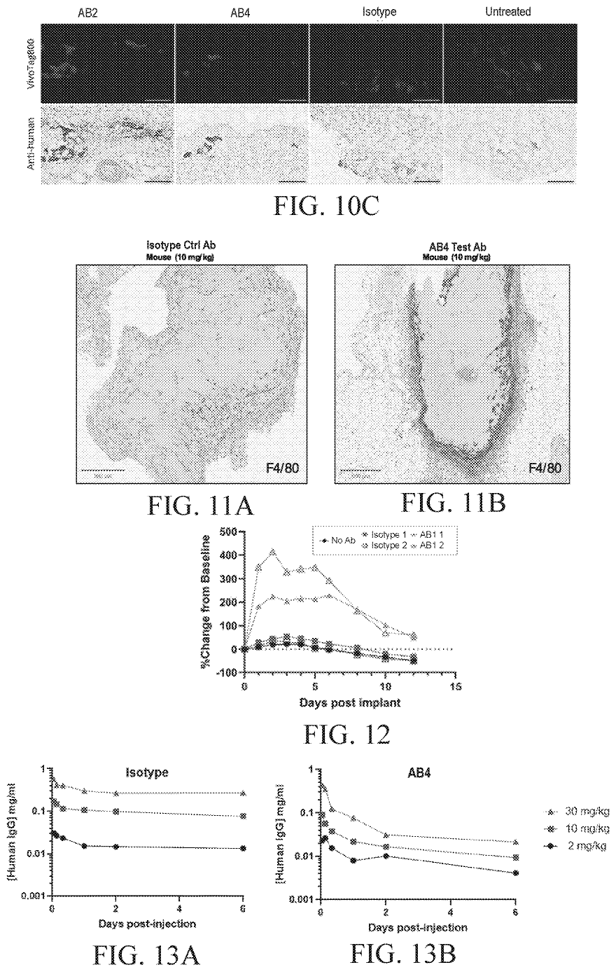

In a separate experiment, we further assessed the colocalization of the AB4 antibody to κ light chain amyloid aggregates in excised amyloidomas. Mice were first subcutaneously implanted with 0.5 mg of κ light chain amyloid proteins to form amyloidomas and were then administered a 10 mg/kg human IgG isotype control or 10 mg/kg of AB4 antibody via tail vein injection. Amyloidomas were then excised from mice, fixed in formalin, and embedded in paraffin prior to staining with biotinylated goat anti-human IgG to detect presence of human IgG in the κ light chain amyloid after tail vein infusion of either 10 mg/kg of human IgG isotype control (FIG. 10A) or 10 mg/kg of AB4 antibody (FIG. 10B). In mice treated with the isotype control, no colocalization of human antibody with κ light chain amyloid aggregates was observed (FIG. 10A). In contrast, amyloidomas excised from mice treated with the AB4 antibody displayed numerous puncta indicating positive colocalization of κ light chain amyloid aggregates with AB4 (arrows). Taken together, these data demonstrate that the anti-human κ light chain antibodies can target and bind to κ light chain amyloid aggregates with great specificity in vivo.

The in vivo targeting of amyloidomas by AB2 and AB4 was also assessed post hoc by measuring VivoTag800-AL09 fibril immunofluorescence and AB2, AB4, and isotype antibody HRP staining in excised amyloidomas from injected mice 4 days after treatment. Both AB2 and AB4 specifically co-localized to the amyloid deposits in the amyloidomas, while the isotype control antibody exhibited non-specific binding to surrounding tissues (FIG. 10C).

Taken together, these data demonstrate the fast-acting and potent ability of the anti-kappa light chain antibodies described herein for reducing and clearing misfolded KLCs, making these antibodies attractive therapeutics for amyloidosis.

### Example 4—Anti-Human κ Light Chain Antibodies Enhance Recruitment of Macrophages to κ Light Chain Amyloid and Elicit Antibody-Dependent Phagocytosis

We next assessed whether treatment with the anti-human κ light chain antibodies triggered an immune response in mice harboring amyloidomas. To test this possibility, we subcutaneously injected mice with 0.5 mg of κ light chain amyloid aggregates and then subsequently administered a 10 mg/kg dose of either human IgG isotype control or AB4 antibody via tail vein infusion. After 4 days following administration of the isotype control or AB4 antibody, amyloidomas were excised, fixed in formalin, paraffin-embedded, and stained with an anti-F4/80 antibody to specifically detect the presence of macrophages surrounding or within the amyloidoma. A histological image of an amyloidoma tissue section obtained from a mouse treated with isotype control displayed a diffuse distribution of F4/80+ cells throughout the amyloidoma (FIG. 11A). In contrast, a high density of F4/80+ cells clearly surrounded and penetrated throughout the amyloidoma within the tissue section obtained from a AB4 antibody-injected mouse (FIG. 11B). These data indicate that treatment with the anti-human κ light chain antibodies effectively induce an immune response in mammals that causes dense recruitment of phagocytic cells, such as macrophages, to κ light chain amyloid aggregates, likely to facilitate the phagocytosis and removal of these toxic protein aggregates from tissue. In a separate experiment, we performed an in vivo phagocytic uptake assay to further characterize whether the anti-human κ light chain antibodies facilitated cellular uptake of κ light chain amyloid aggregates. Briefly, mice received a subcutaneous injection of pHrodo-labelled κ light chain protein (0.5 mg) and were then further infused with either 10 mg/kg of human IgG isotype control or 10 mg/kg of the AB1 antibody. The fluorescence intensity of pHrodo-labelled κ light chain aggregates was measured daily over a 12-day longitudinal imaging time course, and the phagocytic uptake of the pHrodo-labelled κ light chain aggregates was quantified as the percent change from baseline pHrodo fluorescence detected at day 0 (FIG. 12). These data revealed that the AB1 monoclonal antibody induced robust antibody-dependent phagocytosis (ADCP) of κ light chain aggregates in comparison to treatment with the isotype control. For example, ABI treatment resulted in ADCP that was increased by about 200% and sustained from days 1 to 6 in comparison to the isotype control. From days 8 to 12, ADCP gradually diminished and plateaued at a 50% increase in pHrodo fluorescence compared to isotype control. Treatment with the isotype control resulted in a negligible change in the pHrodo fluorescence measured throughout the entire 12-day imaging time course, and thus did not induce ADCP. Taken together, these data indicate that administration of a single dose of the AB1 antibody can induce a long-lived immune response that triggers ongoing ADCP of κ light chain aggregates. And, more generally, these experiments further provide that the anti-human κ light chain antibodies of this disclosure can recruit phagocytic cells, such as macrophages, to clear toxic κ light chain aggregates from a subject.

### Example 5—Anti-Human κ Light Chain Antibodies are Rapidly Cleared from Serum and Recruited to K Aggregates In Vivo

To determine whether the anti-human κ light chain antibodies are effectively recruited from the blood to target κ light chain aggregates in tissues, we tested performed a pharmacokinetic experiment of the clearance rate of the AB4 antibody from serum in mice. Briefly, mice were initially injected subcutaneously with 0.5 mg of κ light chain aggregates in Matrigel to create amyloidomas. The mice were then intravenously injected with either human IgG1 isotype control or AB4 test antibody at 2 mg/kg, 10 mg/kg, or 30 mg/kg. Serum samples from each mouse were collected at 3, 6, 12, 24, 48, 72, and 96 hours after injection of human IgG1 isotype control (FIG. 13A) or AB4 (FIG. 13B). Samples were analyzed for human IgG levels using a Mesoscale discovery (MSD)-based ELISA platform. These data demonstrated that the AB4 antibody was cleared from serum samples at a rate of at least 1.5-fold, 2.5-fold, and 5.5-fold faster than the human IgG1 isotype control over a 2-hour period following administration of the human IgG isotype control and AB4 antibody at 2 mg/kg, 10 mg/kg, and 30 mg/kg, respectively.

### Example 6—Anti-Human κ Light Chain Antibodies Decrease Size of κ Light Chain Amyloid in a Mouse Model of Amyloidoma

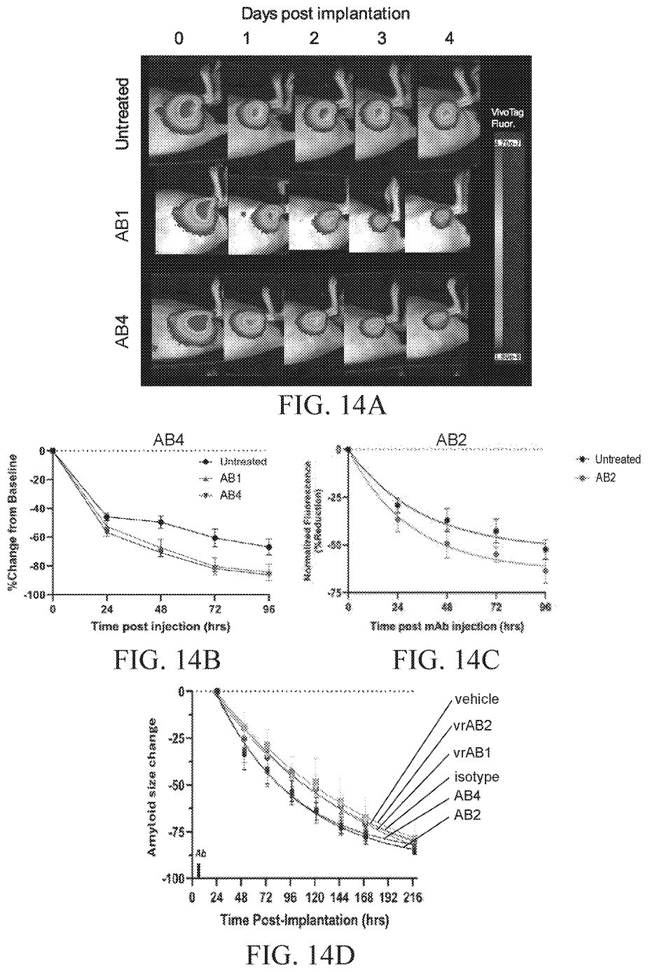

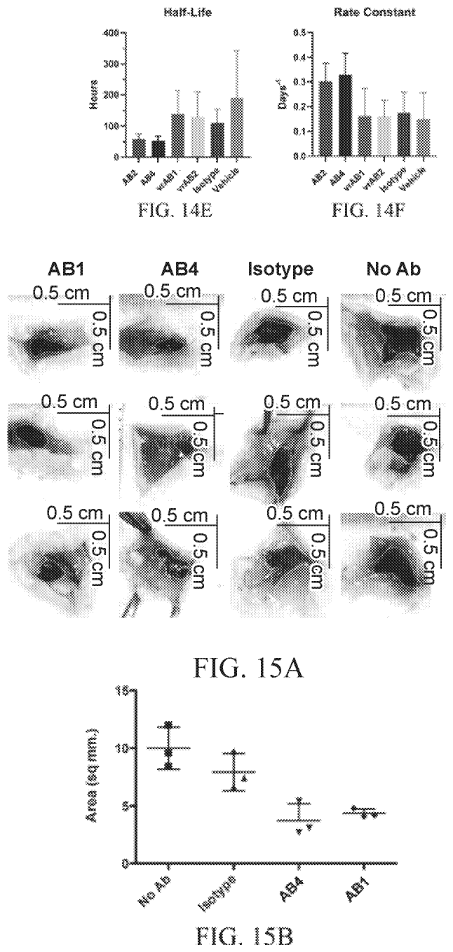

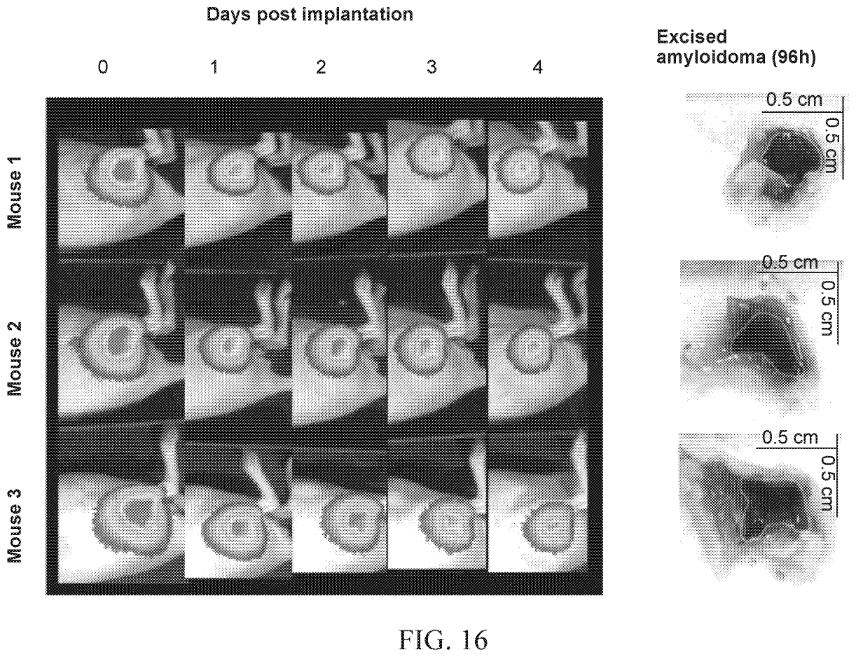

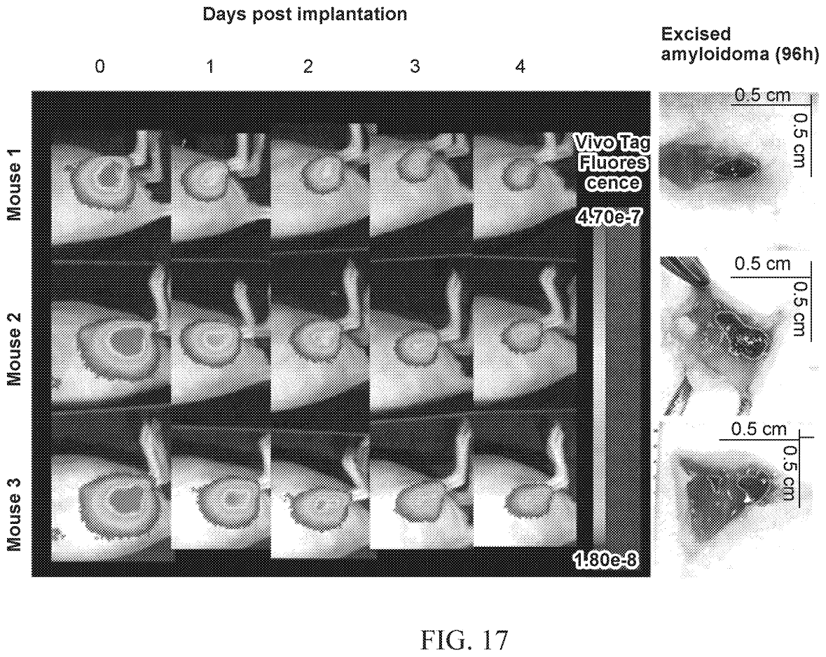

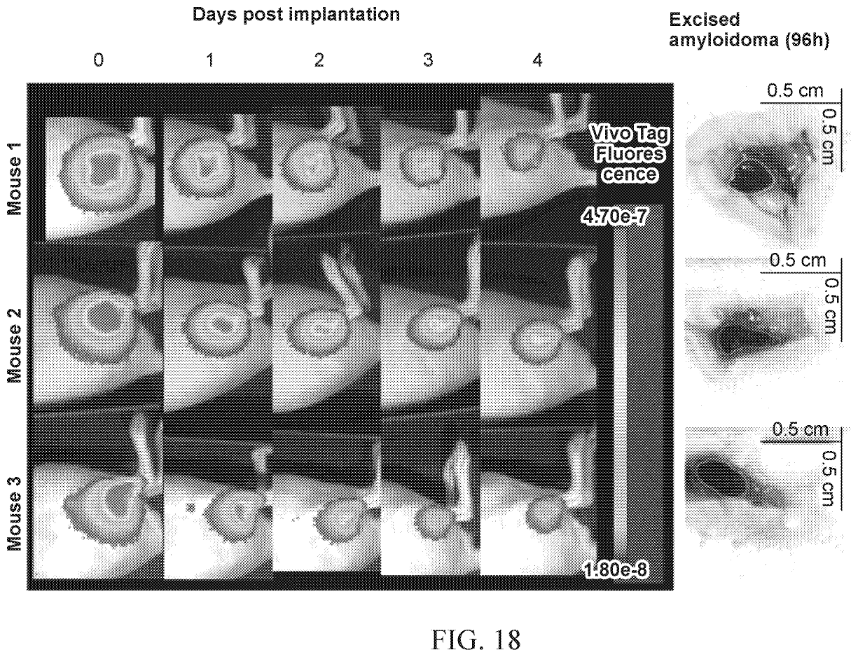

Given their high binding specificity, effective recruitment of macrophages, and robust induction of ADCP, we assessed whether the anti-human κ light chain antibodies could reduce the size of amyloidoma in rodent test subjects. Mice first received an initial subcutaneous injection of VivoTag-800-labelled AL09 κ light chain amyloidoma (0.5 mg), and an initial fluorescent image was acquired to measure the baseline level of total VivoTag-800 fluorescence in each mouse (0 hours). After 6 hours following AL09 amyloidoma implantation, mice were then either further administered a single 30 mg/kg tail vein infusion of AB1 or AB4 antibodies or did not receive any intravenous antibody injection (untreated, no antibody). Throughout the longitudinal imaging study, the total VivoTag-800 fluorescence was measured every 24 hours over the 96-hour time course and quantified as the percent change compared to baseline (0 hours). Following an initial 24-hour wash-out period, the imaging data showed that the size of the amyloidoma stabilized in the untreated animals throughout the 96-hour imaging time course but, in contrast, the amyloidoma size was significantly reduced in mice treated with either the AB1, AB2, or AB4 antibody (FIG. 14A-14C). After 48 hours, the AB1, AB2, and AB4 groups exhibited approximately a mean 70% reduction in total VivoTag-800 fluorescence, while the untreated group displayed only about an average 50% reduction. Moreover, the AB1, AB2, and AB4 groups displayed about an average 60-90% reduction in total VivoTag-800 fluorescence, but the signal measured in the untreated group was reduced by only about 70%. Three mice underwent longitudinal imaging in each of the untreated (FIG. 16), AB4 (FIG. 17), and AB1 (FIG. 18) groups.

Our study also demonstrated that the rate of AL09 amyloid reduction of the constant region-targeting AB2 and AB4 antibodies proved to clear AL09 amyloid levels at a more robust rate compared to the variable region-targeting vrAB1 and vrAB2 antibodies. Here, all test animals were intravenously administered one 30 mg/kg dose of AB2, AB4, vrAB1, vrAB2, or isotype antibodies after subcutaneous injection of AL09 amyloid. Amyloid size change was measured in vivo by VivoTag-AL09 fibril fluorescence in mice treated with vehicle or with a 30 mg/kg dose of AB2, AB4, vrAB1, vrAB2, or isotype antibodies at 0, 24, 48, 72, 96, 120, 144, 168, 192, and 216 hours post-treatment (FIG. 14D). The average half-life of AL09 amyloid fibrils was measured to be ~2- to 3-fold shorter in animals treated with AB2 or AB4 compared to both vrAB1 and vrAB2 (FIG. 14E). The measured rate constant of AL09 amyloid decay rate was decreased by ~2-fold following AB2 or AB4 treatment compared to injection of vrAB1 or vrAB2 (FIG. 14F).

In another experiment, the effect of AB1 or AB4 antibody treatment on amyloidoma size was investigated. Briefly, mice were subcutaneously implanted with AL09 amyloidomas and then either received no antibody (untreated), a human IgG isotype control, AB1 antibody, or AB4 antibody via tail vein injection. Amyloidomas were then acutely imaged under a bright-field surgical microscope 96 hours after implantation (FIG. 15A), and the size (area in mm²) of each amyloidoma was quantified based on the boundaries drawn around the perimeter of the amyloidomas. These data demonstrated that the average amyloidoma size of AB1 and AB4 treatment groups was significantly reduced by more than 50% compared to the untreated group and by about 40-50% compared to the isotype control group (FIG. 15B). While isotype-treated animals showed a slight reduction in amyloidoma size, it was not statistically significant compared to the untreated animals. Images of excised amyloidomas of mice in the untreated (FIG. 16), AB4 (FIG. 17), and ABI (FIG. 18) groups. Each group consisted of 3 animals. Taken together, the anti-human κ light chain antibodies effectively reduce amyloidoma size after systemic delivery of only a single dose, which indicates great therapeutic potential for treating conditions or disorders related to antibody light chain aggregation (e.g., κ light chain aggregation), such as antibody light chain amyloidosis.

### Example 7—Dose-Dependent Clearance of Misfolded κ Light Chain Aggregates by Anti-Human κ Light Chain Antibodies

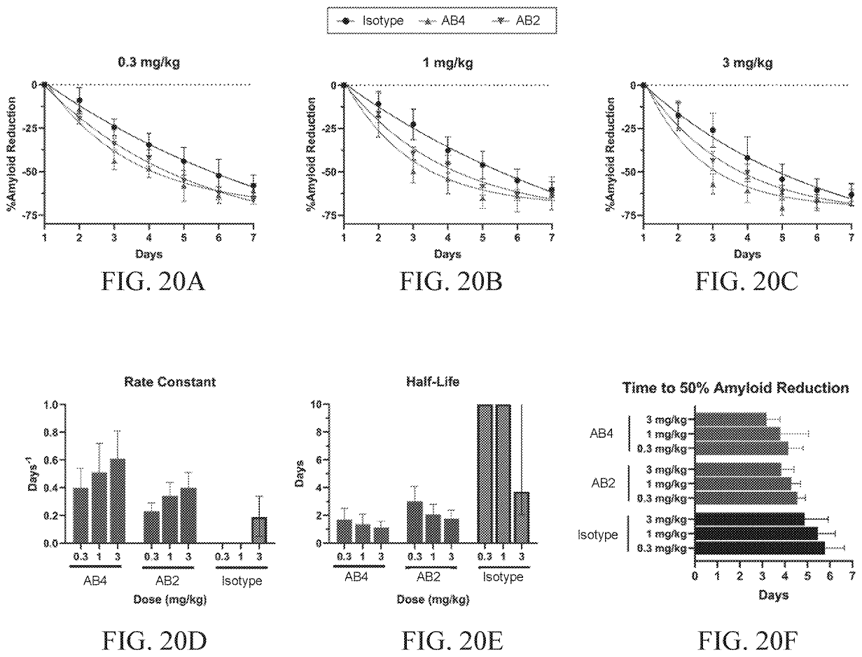

Given the rapid and robust clearance of κ light chain aggregates by the anti-κ light chain antibodies described herein, we next examined the effectiveness of ABI, AB2, and AB4 on clearing AL09 κ light chain aggregates at lower doses: 0.3 mg/kg, 1 mg/kg, and 30 mg/kg. As described in EXAMPLE 5 and EXAMPLE 6, test animals were subcutaneously implanted with VivoTag800-tagged AL09 fibrils. After implantation, test animals received a single intravenous injection of either the test anti-κ light chain antibodies or isotype control antibody, and in vivo fluorescence imaging of VivoTag800-AL09 fibril fluorescence was then measured daily. Here, we first measured VivoTag800-AL09 fibrils from days 0 to 7 following treatment with a single dose of either isotype, AB4, or AB2 at 0.3 mg/kg (FIG. 20A), 1 mg/kg (FIG. 20B), or 3 mg/kg (FIG. 20C).

After imaging was completed in the amyloidoma mouse model, amyloid reduction curves were extrapolated from VivoTag800-tagged AL09 fibril fluorescence from days 1-7 post-treatment and were fit using the exponential decay function of Equation I to determine decay rate and amyloidoma half-life to determine decay rate and amyloidoma half-life of Equation II.

N(t)=N₀e^{-λt} Equation I:

t_{1/2}=ln(2)/λ Equation II:

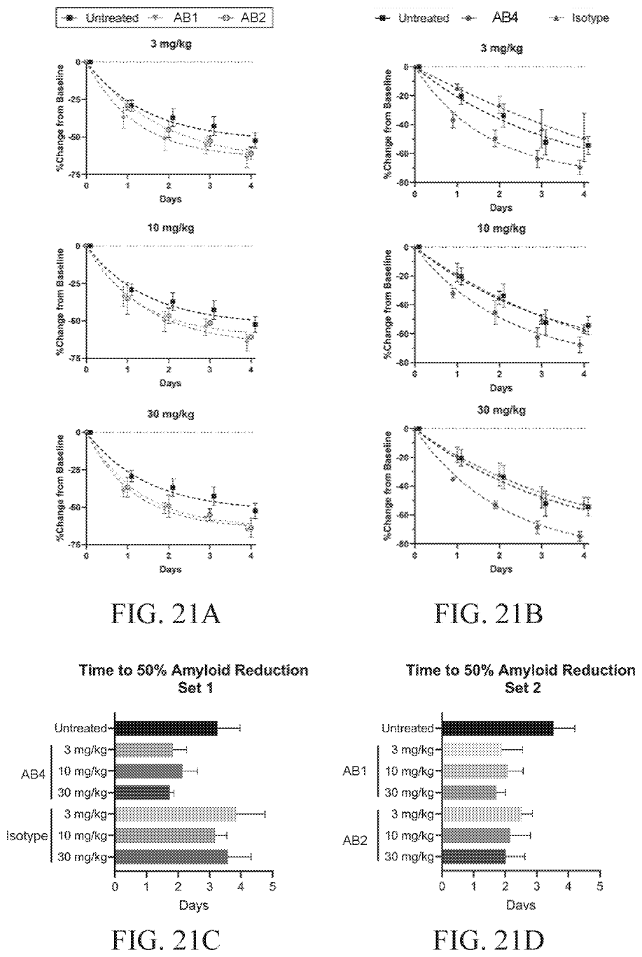

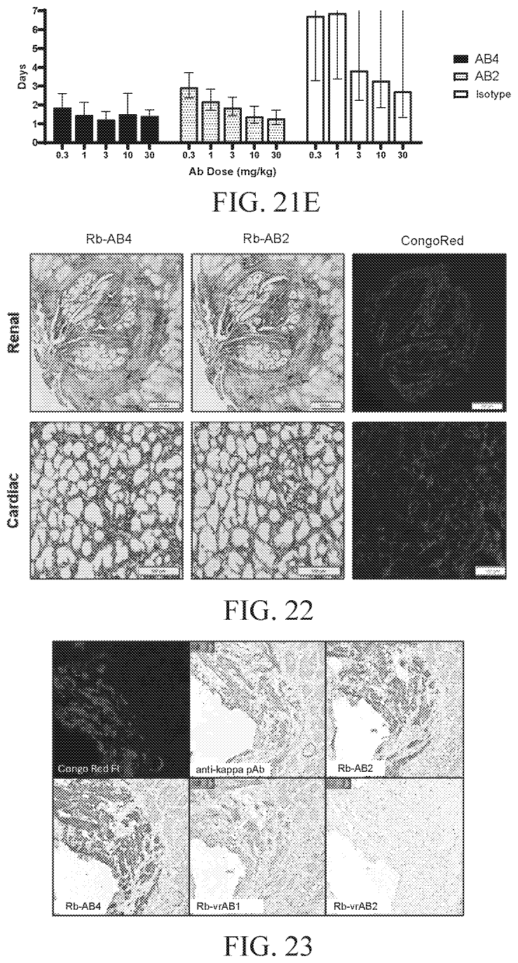

These data revealed that AB2 and AB4 increased the rate of κ light chain aggregate reduction at all tested doses compared to isotype control at all doses (FIGS. 20A-20C). Based on the amyloid reduction curves at the tested doses, the decay rate constant of κ light chain aggregates following treatment with AB2 or AB4 ranged from ~0.2-0.6 (FIG. 20D). The calculated average half-life of amyloid reduction by AB2 or AB4 was ~1-3 days compared to ~8 days in the isotype-treated group (FIG. 20E), or was 3.5 days on average across 0.3, 1, 3, 10, 30 mg/kg doses of AB2 or AB4 compared to −5 days with isotype treatment (FIG. 21E). AB2- and AB4-treated animals exhibited an average time to 50% amyloid reduction of ~3.5 days compared to the −5 days in the isotype-treated animals (FIG. 20F).

In the same amyloidoma mouse model, we tested additional single-dose regimens (3, 10, and 30 mg/kg) of by treating animals intravenously with ABI, AB2, or AB4 on amyloidoma reduction compared to untreated and isotype-treated control groups. Our results demonstrated that all doses of either AB1, AB2 or AB4 effectively increased the rate of amyloidoma reduction compared to isotype control at all doses (FIG. 21A-21B). All antibodies AB1, AB2, and AB4 at all tested doses were able to reduce AL09 amyloid levels by at least 50% over a time period of approximately ~1-2 days after treatment (FIG. 21C, FIG. 21D).

### Example 8—Specific Binding to κ Light Chain Aggregates by Anti-Human κ Light Chain Antibodies in Tissue of Light Chain Amyloidosis Patients

We next examined the binding of the anti-κ light chain antibodies or κ light chains of this disclosure to κ light chain aggregates in human tissue. We obtained tissue biopsies from patients with confirmed Light chain amyloidosis (AL amyloidosis). Tissues samples were from patient kidneys, heart, and colon and sectioned for immunohistochemical (IHC) analysis. Here, the AB2, AB4, and the vrAB1 and vrAB2 antibodies were reformatted to a rabbit Fc region to minimize human-Fc-associated off-target binding. IHC was then conducted using both AB2 and AB4 to evaluate targeting of κ light chain aggregates in biopsy confirmed AL patient tissue. Evaluation of IHC staining intensity was based upon assumed uniformity and minimum variability in the staining protocols, and the same antibody concentrations using automated staining with the same reagents and protocol were used for each tested antibody. Therefore, differences in intensity of IHC stain within this study were considered to be associated with binding characteristics of the primary antibodies to tissue antigens in tested tissue sections.

Glomerular staining of renal tissue and staining of the intramuscular space of transverse sections of heart muscle fibers were observed, demonstrating on-target binding to amyloid LC in patient tissues by AB2 and AB4 (FIG. 22). Briefly, 4 μm-thick sections were stained with AB2 and AB4 antibodies that were reformatted with rabbit fragment crystallizable (Fc) domains as the primary antibody, followed by HRP-conjugated anti-rabbit secondary antibodies. DAB was used for colorimetric detection of HRP, and renal and cardiac tissue were counterstained with hematoxylin (blue). IHC staining of AB2 and AB4 signal highly colocalized with the fluorescent signal of the amyloid-specific dye, Congo Red (FIG. 22).

Misfolded κ light chain staining was assessed in kidney hilus tissue sections with rabbit-chimerized AB2 and AB4 antibodies, rabbit-chimerized variable region-targeting anti-κ LC antibodies vrAB1 and vrAB2, and a phospho-specific anti-kappa light chain antibody (anti-κ pAb), the latter serving as a positive control. As shown in FIG. 23, AB2 and AB4 exhibited robust staining of misfolded κ light chains in hilus tissue, while vrAB2 had weak positive staining with low signal over background. No measurable detection was observed with the vrAB2 antibody.

The anti-kappa light chain antibodies were also tested in colon sections, whereby AB2 and AB4 again produced robust and definitive detection of misfolded κ light chains (FIG. 24). In contrast, vrAB1 exhibited low discrimination between amyloid-positive and amyloid-negative regions of the tissue section, and minimal amyloid signal was observed with vrAB2 (FIG. 24).

Detection in synovium tissue sections similarly showed AB2 and AB4 antibodies with robust misfolded amyloid signal and high colocalization with Congo Red staining, while vrAB1 demonstrated negative amyloid staining (FIG. 25).

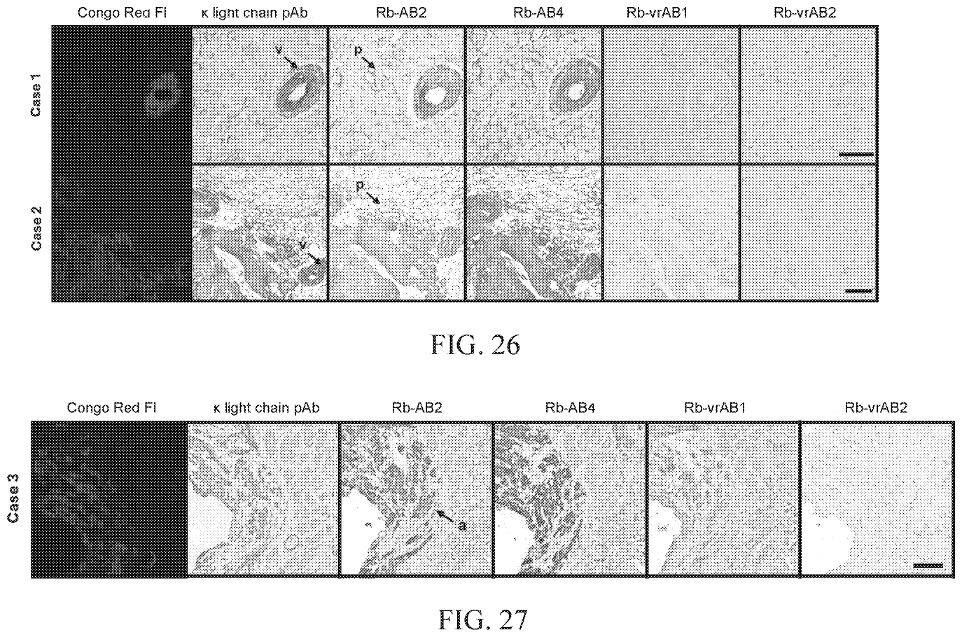

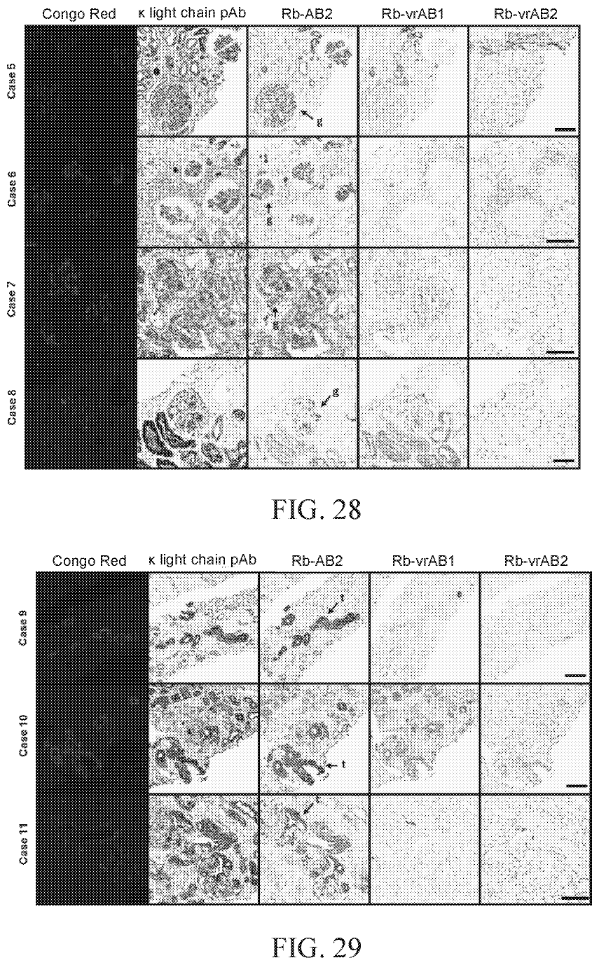

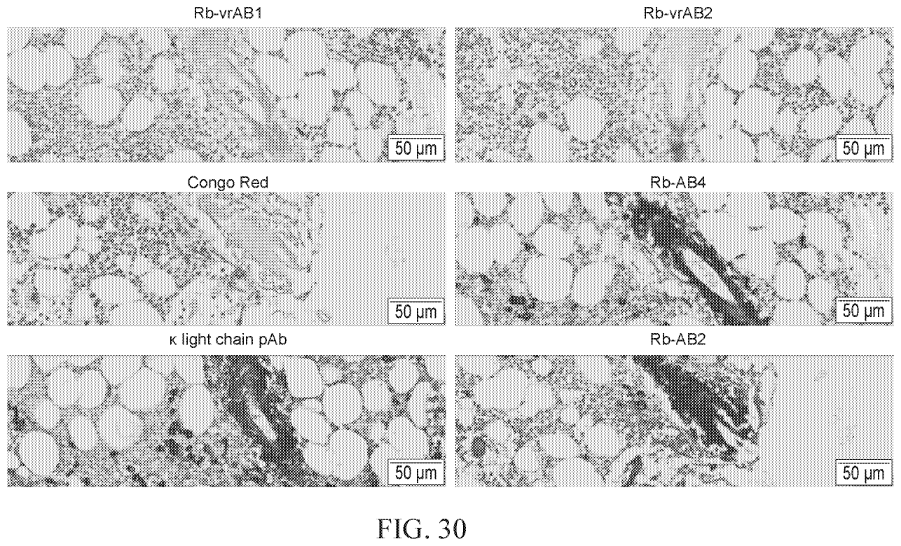

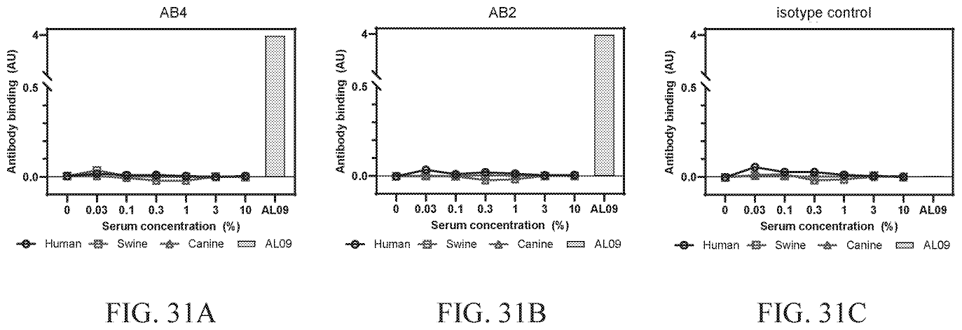

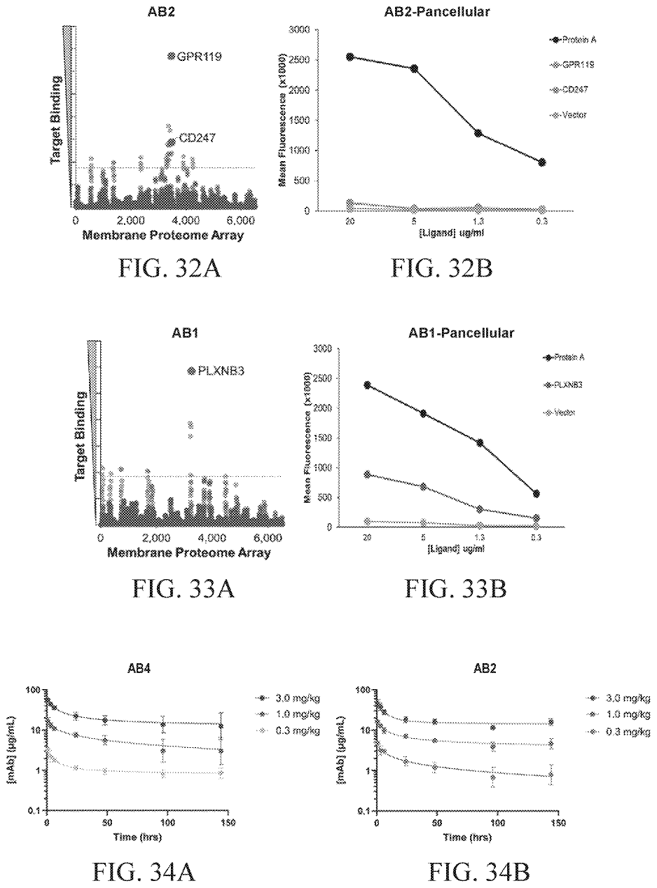

In heart tissue sections, AB2 and AB4 exhibited on-target binding (high colocalization with Congo Red signal) in vascular amyloid (v) and interstitial pericellular amyloid (p) with lower background staining compared to the anti-kappa pAb control (FIG. 26). The vrAB1 antibody was demonstrated to have negative amyloid staining and high background, and vrAB2 showed negative staining with low background (FIG. 26). Similar staining patterns were observed in kidney tissue hilus, glomerulus, and tubule); clear amyloid staining was apparent with constant region-targeting AB2 and/or AB4, but vrAB1 exhibited low specificity and low signal intensity (FIG. 27-29). Amyloid staining was weak or essentially absent with vrAB1 (FIG. 27-29). As shown in FIG. 3O, AB4 and AB2 exhibited strong straining signal that was comparable to the phospho-specific anti-kappa light chain antibody positive control and, notably, was clearly more intense than both vrAB1 and vrAB2 antibodies (FIG. 3O). The vrAB1 and vrAB2 antibodies detected very minimal amyloid staining in bone marrow.

TABLE 5 shows a comparative summary of IHC staining between AB2 and AB4 versus vrAB1 and vrAB2 detected in amyloidosis patient tissue samples of spleen, heart, kidney, lung, colon, GI tract, skin, subcutis, soft tissue, tendon, bone, synovium, gall bladder, nasopharynx, and lymph node. Amyloid signal by Congo Red staining was present in all tested samples. AB2, AB4, and vrAB1 antibodies showed similar staining patterns in the lung, GI tract, skin, subcutis, soft tissue, tendon, bone, gall bladder, nasopharynx, and lymph nodes. However, vrAB1 generally exhibited lower intensity and lower signal-to-background signal compared to AB2 and AB4. vrAB2 exhibited generally weak binding to all tissues. AB2 and AB4 exhibited reliable staining signal and greater intensity and stronger differentiation of amyloid over background patterns in heart, kidney, lung, colon, soft tissue, synovium, and spleen compared to vrAB1 and vrAB2, revealing κLC amyloid distribution that was not visualized by, or staining signal that did not colocalize with, vrAB1 and vrAB2. Among all 36 samples tested, AB2-mediated amyloid staining was detected in 100% of tissue samples, while vrAB1 demonstrated similar amyloid staining in only 50% of tissue samples. The sensitivity of amyloid detection was significantly increased with AB32 than vrAB1 (p=4.38×10⁻⁷**, Fisher's exact test for two proportions).

TABLE 5
IHC detection of κLC amyloid by anti-κ light chain antibodies or
anti-κLC antibodies in various tissues of amyloidosis patients

| | | | | | | Staining |
|---|---|---|---|---|---|---|
| | | | | | | pattern |
| | | AB2 | AB4 | vrAB1 | vrAB2 | of AB2 |
| | | amyloid | amyloid | amyloid | amyloid | or AB4 |
| Tissue | Amyloid | signal | signal | signal | signal | vs vrAB1 |
| Heart | Present | ++ | ++ | − | − | Improved |
| Kidney | Present | ++ | ++ | +/− | − | Improved |
| Lung | Present | +/− | +/− | +/− | − | = |
| Lung | Present | w+ | w+ | +/− | − | Improved |
| Lung | Present | +/− | +/− | +/− | − | = |
| Lung | Present | +/− | +/− | +/− | + | = |
| Lung | Present | + | + | + | − | = |
| Colon | Present | + | + | +/− | − | Improved |
| GI | Present | +/− | +/− | +/− | w+ | = |
| Colon | Present | + | + | + | − | = |
| Colon | Present | NA | +/− | +/− | − | = |
| Skin | Present | +/− | +/− | +/− | − | = |
| Subcutis | Present | +/− | +/− | +/− | − | = |
| Soft | Present | +/− | +/− | +/− | − | = |
| Tissue | | | | | | |
| Soft | Present | + | + | +/− | − | Improved |
| Tisue | | | | | | |
| B-cell | Present | +/− | +/− | +/− | − | = |
| Tumour | | | | | | |
| Bone | NA | NA | NA | NA | NA | = |
| Synovium | Present | + | + | − | − | Improved |
| Bone | Present | + | + | + | w+ | = |
| Gall | Present | + | + | + | w+ | = |
| Bladder | | | | | | |
| Naso- | Present | + | + | + | + | = |
| pharynx | | | | | | |
| Spleen | Present | ++ | ++ | + | w+ | Improved |
| Lymph | Present | + | + | + | − | = |
| Node | | | | | | |
| Heart | Present | ++ | ++ | w+ | +/− | Improved |
| Bone | Present | + | + | w+ | w+ | Improved |
| Skin | Present | + | + | + | − | = |
| Kidney | Present | NA | ++ | w+ | − | Improved |
| Kidney | Present | NA | ++ | w+ | − | Improved |
| Kidney | Present | NA | ++ | w+ | w+ | Improved |
| Kidney | Present | NA | + | w+ | − | Improved |
| Kidney | Present | NA | ++ | + | + | Improved |
| Kidney | Present | NA | + | − | − | Improved |
| Kidney | Present | NA | + | w+ | +/− | Improved |
| Heart | Present | NA | + | + | +/− | = |
| Tongue | Present | ++ | ++ | + | w+ | Improved |
| Kidney | Present | NA | ++ | + | w+ | Improved |

− = negative
+/− = positive with partial concordance with Congo Red
w+ = weak positive
+ = positive
++ = strong positive
NA = Not Available

Taken together, the constant region-targeting anti-κ light chain antibodies described herein can reliably detect misfolded κ light chains in several human tissues with high signal-to-noise and out-performed variable-region binding kappa LC antibodies.

### Example 9—Screen for Non-Specific Binding Targets of Anti-Human κ Light Chain Antibodies In Vitro

We next examined whether the anti-κ light chain antibodies could have any non-specific binding to targets other than misfolded κ light chains. Indirect ELISA showed that no binding of AB4 or AB2 to soluble human (non-AL amyloidosis), swine, or canine serum proteins was observed (FIGS. 31A-31C). As expected, AB2 and AB4 exhibited robust binding to AL09 patient samples (positive control for misfolded κ light chain amyloid), and minimal-to-absent binding to the isotype antibody was observed in the tested samples.

ABI and AB2 for potential off-target binding using Integral Molecular's Membrane Proteome Array (MPA) with a protein library of 6000 human proteins human membrane protein clones, each overexpressed in live cells from expression plasmids. Each clone was individually transfected in separate wells of a 384-well plate followed by a 24-hour incubation. Cells expressing each individual MPA protein clone were arrayed in duplicate in a matrix format for high-throughput screening. Each test antibody was added to the MPA at the predetermined concentration, and binding across the protein library was measured on an Intellicyt iQue using a fluorescently labeled secondary antibody. Each array plate contains both positive (Fc-binding) and negative (empty vector) controls for plate-by-plate reproducibility. Test ligand interactions with any targets identified by MPA screening were confirmed in a second flow cytometry experiment using serial dilutions of each test antibody, and the target identity was re-verified by sequencing. Although MPA identified 2 potential binders—GRP119 and CD247—to AB2, their binding signals in the validation flow cytometry assay were very low (FIG. 32A, FIG. 32B). Only one potential binder to ABJ-PLXNB3—was detected by MPA screening, which exhibited low binding in the flow cytometry assay (FIG. 33A, FIG. 33B). These data demonstrate the high specificity of the anti-κ light chain antibodies provided herein for binding misfolded c light chains.

### Example 10—Pharmacokinetics of Anti-Human κ Light Chain Antibodies

### Dose-Responsive Amyloid Clearance by Anti-Kappa Light Chain Antibodies In Vivo

The clearance rate of AB2 and AB4 antibodies from blood plasma following intravenous administration of a single 0.3 mg/kg, 1 mg/kg, or 3 mg/kg dose of either AB4 and AB2 was also measured in mice. After administration of each antibody, AB2 and AB4 levels in blood samples were evaluated at 0, 4, 8, 24, 48, 96, and 144 hours post-dose (FIG. 34A, FIG. 34B). Diluted serum samples were captured on plates coated with anti-human-Fc capture antibody and subsequently detected using anti-human-Fab antibody tagged with MSD Gold Sulfo-Tag and red. Antibody concentrations were extrapolated based on standard curves constructed using known dilutions of pure antibody. AB2 and AB4 exhibited comparable pharmacokinetic parameters (TABLE 6). As shown in TABLE 6, the mean observed maximum concentration (Cₘₐₓ) between AB2 and AB4 was observed at 0.5 hour post-dose, showing a dose-dependent increase from 3.3 pg/mL to 56.6 pg/mL for AB4 and from 4.9 pg/mL to 47.8 pg/mL for AB2. The mean area under the plasma concentration time curve (AUC) increased from 0 to 144 hours post dose (AUC₀₋₁₄₄ₕ) also demonstrated a dose-dependent increase from 151.3 h×μg/mL to 2776.1 h×μg/mL for AB4 and from 166.3 h×μg/mL to 2388.7 h×μg/mL for AB2.

TABLE 6
Mean PK Parameters of AB2 and AB4
in Mice Following a Single IV Dose

| | IV Dose | | |
|---|---|---|---|
| | 0.3 mg/kg | 1.0 mg/kg | 3.0 mg/kg |
| Pharmacokinetic | Mean ± SD | Mean ± SD | Mean ± SD |
| Parameters | N = 5 | N = 5 | N = 5 |

AB4

| | | | |
|---|---|---|---|
| Cₘₐₓ (μg/mL) | 3.3 ± 0.4 | 17.1 ± 3.0 | 56.6 ± 3.3 |
| AUC_{0-144 h} (h · μg/mL) | 151.3 ± 23.6 | 808.0 ± 187.3 | 2776.1 ± 668.7 |
| Tₘₐₓ (h) | 0.5 | 0.5 | 0.5 |

AB2

| | | | |
|---|---|---|---|
| Cₘₐₓ (μg/mL) | 4.9 ± 0.5 | 16.5 ± 1.2 | 47.8 ± 10.0 |
| AUC0-144 h | 166.3 ± 42.6 | 822.3 ± 96.4 | 2388.7 ± 260.2 |
| Tₘₐₓ (h) | 0.5 | 0.5 | 0.5 |

AUC_{0-144 h} = area under the plasma concentration-time curve from time 0 to 144 hours post-dose; Cₘₐₓ = observed maximum concentration; IV = intravenous; N = number of animals tested; PK = pharmacokinetics; SD = standard deviation; Tmax = time to observed maximum concentration.

While preferred embodiments of the present within a composition disclosure have been shown and described herein, it will be obvious to those skilled in the art that such embodiments are provided by way of example only. Numerous variations, changes, and substitutions will now occur to those skilled in the art without departing from the instant disclosure. It should be understood that various alternatives to the embodiments described herein may be employed in practicing the invention. It is intended that the following claims define the scope of the embodiments disclosed herein, and that methods and structures within the scope of these claims and their equivalents be covered thereby.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

1. An anti-human kappa light chain antibody or a human kappa light chain binding fragment thereof comprising:
(a) heavy chain complementarity determining region 1 (H-CDR1) comprising the amino acid sequence set forth in SEQ ID NO: 2; a heavy chain complementarity determining region 2 (H-CDR2) comprising the amino acid sequence set forth in SEQ ID NO: 3; a heavy chain complementarity determining region 3 (H-CDR3) comprising the amino acid sequence set forth in SEQ ID NO: 4; a light chain complementarity determining region 1 (L-CDR1) comprising the amino acid sequence set forth in SEQ ID NO: 6; a light chain complementarity determining region 2 (L-CDR2) comprising the amino acid sequence set forth in SEQ ID NO: 7; and a light chain complementarity determining region 3 (L-CDR3) comprising the amino acid sequence set forth in SEQ ID NO: 8;
(b) an H-CDR1 comprising the amino acid sequence set forth in SEQ ID NO: 10; an H-CDR2 comprising the amino acid sequence set forth in SEQ ID NO: 11; an H-CDR3 comprising the amino acid sequence set forth in SEQ ID NO: 12; an L-CDR1 comprising the amino acid sequence set forth in SEQ ID NO: 14; an L-CDR2 comprising the amino acid sequence set forth in SEQ ID NO: 15; and the L-CDR3 comprising the amino acid sequence set forth in SEQ ID NO: 16.
(c) an H-CDR1 comprising the amino acid sequence set forth in SEQ ID NO: 18; an H-CDR2 comprising the amino acid sequence set forth in SEQ ID NO: 19; an H-CDR3 comprising the amino acid sequence set forth in SEQ ID NO: 20; an L-CDR1 comprising the amino acid sequence set forth in SEQ ID NO: 22; an L-CDR2 comprising the amino acid sequence set forth in SEQ ID NO: 23; and an L-CDR3 comprising the amino acid sequence set forth in SEQ ID NO: 24; or
(d) an H-CDR1 comprising the amino acid sequence set forth in SEQ ID NO: 26; an H-CDR2 comprising the amino acid sequence set forth in SEQ ID NO: 27; an H-CDR3 comprising the amino acid sequence set forth in SEQ ID NO: 28; an L-CDR1 comprising the amino acid sequence set forth in SEQ ID NO: 30; an L-CDR2 comprising the amino acid sequence set forth in SEQ ID NO: 31; and an L-CDR3 comprising the amino acid sequence set forth in SEQ ID NO: 32.

2. The anti-human kappa light chain antibody or the human kappa light chain binding fragment thereof of claim 1, further comprising:
(a) a heavy chain variable region comprising the amino acid sequence of SEQ ID NO: 1; and
(b) alight chain variable region comprising the amino acid sequence of SEQ ID NO: 5.

3. The anti-human kappa light chain antibody or the human kappa light chain binding fragment thereof of claim 1, further comprising:
(a) a heavy chain variable region comprising the amino acid sequence of SEQ ID NO: 9; and
(b) alight chain variable region comprising the amino acid sequence of SEQ ID NO: 13.

4. The anti-human kappa light chain antibody or the human kappa light chain binding fragment thereof of claim 1, further comprising:
(a) a heavy chain variable region comprising the amino acid sequence of SEQ ID NO: 17; and
(b) alight chain variable region comprising the amino acid sequence of SEQ ID NO: 21.

5. The anti-human kappa light chain antibody or the human kappa light chain binding fragment thereof of claim 1, further comprising:
(a) a heavy chain variable region comprising the amino acid sequence of SEQ ID NO: 25; and
(b) alight chain variable region comprising the amino acid sequence of SEQ ID NO: 29.

6. The anti-human kappa light chain antibody or the human kappa light chain binding fragment thereof of claim 1, wherein the human kappa light chain binding fragment comprises a Fab, a Fab′, a F(ab′)₂, a scFv, a dsFv, a ds-scFv, a dimer, a minibody, or a diabody.

7. The anti-human kappa light chain antibody or the human kappa light chain binding fragment thereof of claim 1, wherein the anti-human kappa light chain antibody is an IgG antibody.

8. The anti-human kappa light chain antibody or the human kappa light chain binding fragment thereof of claim 7, wherein the IgG antibody is an IgG1 antibody or an IgG4 antibody.

9. The anti-human kappa light chain antibody or the human kappa light chain binding fragment thereof of claim 1, wherein the anti-human kappa light chain antibody or human kappa light chain binding fragment thereof binds to a kappa light chain aggregate.

10. The anti-human kappa light chain antibody or the human kappa light chain binding fragment thereof of claim 1, wherein the anti-human kappa light chain antibody or the human kappa light chain binding fragment thereof is characterized by a higher binding affinity for a kappa light chain aggregate than a native kappa light chain.

11. A nucleic acid or a plurality of nucleic acids encoding the anti-human kappa light chain antibody or the human kappa light chain binding fragment thereof of claim 1.

12. A cell comprising a nucleic acid or a plurality of nucleic acids encoding the anti-human kappa light chain antibody or the human kappa light chain binding fragment thereof of claim 1.

13. A method of manufacturing the anti-human kappa light chain antibody or the human kappa light chain binding fragment thereof of claim 1, the method comprising culturing a cell comprising a nucleic acid encoding the anti-human kappa light chain antibody or human kappa light chain binding fragment thereof under conditions sufficient to allow production and secretion of the anti-human kappa light chain antibody or the human kappa light chain binding fragment thereof from the cell and isolating or purifying the anti-human kappa light chain antibody or the human kappa light chain binding fragment thereof from the cell or a supernatant of the cell.

14. A method of detecting a kappa light chain aggregate in a sample, the method comprising contacting the sample with the anti-human kappa light chain antibody or the human kappa light chain binding fragment thereof of claim 1.

15. The method of claim 14, wherein the sample comprises blood, serum, plasma, solid tissue, liquid biopsy, bone marrow, cerebrospinal fluid, or any combination thereof.

16. A method of increasing clearance or phagocytosis of a kappa light chain aggregate, the method comprising contacting the kappa light chain aggregate with the anti-human kappa light chain antibody or the human kappa light chain binding fragment thereof of claim 1.

17. A method of treating a disease or condition characterized by kappa light chain aggregates in a subject in need thereof, the method comprising administering the anti-human kappa light chain antibody or the human kappa light chain binding fragment thereof of claim 1 to the subject, thereby treating the disease or condition characterized by kappa light chain aggregates in the subject.

18. The method of claim 17, wherein the disease or condition characterized by kappa light chain aggregates is a plasma cell dyscrasia.

19. The method of claim 18, wherein the plasma cell dyscrasia comprises an amyloidosis, a monoclonal gammopathy of unknown significance (MGUS), multiple myeloma (MM), plasma cell leukemia (PCL), or any combination thereof.

20. The method of claim 17, wherein the disease or condition characterized by kappa light chain aggregates is amyloidosis, systemic amyloidosis, cardiac amyloidosis, renal amyloidosis, or any combination thereof.


SEQUENCE LISTING (33 sequences)

SEQ ID NO: 1 - protein, 119 aa, synthetic construct
EVQLLESGGGLVQPGGSLRLSCAASGFTFSSYGMSWVRQAPGKGLEWVSAISYSGGSTYYADSVKGRFTISRDNSKNTLYLQMNSLRAEDTAVYYCASTGWESDAFDIWGQGTLVTVSS

SEQ ID NO: 2 - protein, 13 aa, synthetic construct
AASGFTFSSYGMS

SEQ ID NO: 3 - protein, 10 aa, synthetic construct
AISYSGGSTY

SEQ ID NO: 4 - protein, 12 aa, synthetic construct
ASTGWESDAFDI

SEQ ID NO: 5 - protein, 107 aa, synthetic construct
DIQMTQSPSSLSASVGDRVTITCRASQGIRNDLGWYQQKPGKAPKLLIYAASSLQTGVPSRFSGSGSGTHFTLTISSLQPDDFATYYCQQGNSFPPTFGQGTKVEIK

SEQ ID NO: 6 - protein, 11 aa, synthetic construct
RASQGIRNDLG

SEQ ID NO: 7 - protein, 8 aa, synthetic construct
YAASSLQT

SEQ ID NO: 8 - protein, 9 aa, synthetic construct
QQGNSFPPT

SEQ ID NO: 9 - protein, 116 aa, synthetic construct
QVQLVQSGAEVKKPGSSVKVSCKASGYTFTSLWIQWVRQAPGQGLEWMGSIYPGDGDTQYIQKFRGRVTITADESTSTAYMELSSLRSEDTAVYYCSIITTAPDYWGQGTLVTVSS

SEQ ID NO: 10 - protein, 13 aa, synthetic construct
KASGYTFTSLWIQ

SEQ ID NO: 11 - protein, 10 aa, synthetic construct
SIYPGDGDTQ

SEQ ID NO: 12 - protein, 9 aa, synthetic construct
SIITTAPDY

SEQ ID NO: 13 - protein, 112 aa, synthetic construct
DVVMTQSPLSLPVTLGQPASISCKSSQDLLDVDGKTYLNWFQQRPGQSPRRLIYHVSKLDSGVPDRFSGSGSGTDFTLKISRVEAEDVGVYYCHQGTHFPQTFGGGTKVEIK

SEQ ID NO: 14 - protein, 16 aa, synthetic construct
KSSQDLLDVDGKTYLN

SEQ ID NO: 15 - protein, 8 aa, synthetic construct
YHVSKLDS

SEQ ID NO: 16 - protein, 9 aa, synthetic construct
HQGTHFPQT

SEQ ID NO: 17 - protein, 116 aa, synthetic construct
QVQLVQSGAEVKKPGSSVKVSCKASGYTFRSQWIQWVRQAPGQGLEWMGSIYPGDGDTQYIQKFRGRVTITADESTSTAYMELSSLRSEDTAVYYCSIITTAPDYWGQGTLVTVSS

SEQ ID NO: 18 - protein, 13 aa, synthetic construct
KASGYTFRSQWIQ

SEQ ID NO: 19 - protein, 10 aa, synthetic construct
SIYPGDGDTQ

SEQ ID NO: 20 - protein, 9 aa, synthetic construct
SIITTAPDY

SEQ ID NO: 21 - protein, 112 aa, synthetic construct
DVVMTQSPLSLPVTLGQPASISCKSSQDLLEGDGKTYLNWFQQRPGQSPRRLIYHVSKLDSGVPDRFSGSGSGTDFTLKISRVEAEDVGVYYCHQGTHFPQTFGGGTKVEIK

SEQ ID NO: 22 - protein, 16 aa, synthetic construct
KSSQDLLEGDGKTYLN

SEQ ID NO: 23 - protein, 8 aa, synthetic construct
YHVSKLDS

SEQ ID NO: 24 - protein, 9 aa, synthetic construct
HQGTHFPQT

SEQ ID NO: 25 - protein, 116 aa, synthetic construct
QVQLVQSGAEVKKPGSSVKVSCKASGYTFRSKWIQWVRQAPGQGLEWMGSIYPGDGDTQYIQKFRGRVTITADESTSTAYMELSSLRSEDTAVYYCSIITTAPDYWGQGTLVTVSS

SEQ ID NO: 26 - protein, 13 aa, synthetic construct
KASGYTFRSKWIQ

SEQ ID NO: 27 - protein, 10 aa, synthetic construct
SIYPGDGDTQ

SEQ ID NO: 28 - protein, 9 aa, synthetic construct
SIITTAPDY

SEQ ID NO: 29 - protein, 112 aa, synthetic construct; identical to SEQ ID NO: 21

SEQ ID NO: 30 - protein, 16 aa, synthetic construct
KSSQDLLEGDGKTYLN

SEQ ID NO: 31 - protein, 8 aa, synthetic construct
YHVSKLDS

SEQ ID NO: 32 - protein, 9 aa, synthetic construct
HQGTHFPQT

SEQ ID NO: 33 - protein, 10 aa, Homo sapiens
STYSLSSTLT